(19) United States
(12) Patent Application Publication
(10) Pub. No.: US 2013/0157261 A1
(43) Pub. Date: Jun. 20, 2013

(54) Compositions and Methods for Quantitative Histology, Calibration of Images in Fluorescence Microscopy, and ddTUNEL Analyses

(71) Applicants: Martyn Alun Sharpe, Houston, TX (US); David S. Baskin, Houston, TX (US)

(72) Inventors: Martyn Alun Sharpe, Houston, TX (US); David S. Baskin, Houston, TX (US)

(73) Assignee: The Methodist Hospital Research Institute, Houston, TX (US)

(21) Appl. No.: 13/487,058

(22) Filed: Jun. 1, 2012

Related U.S. Application Data
(60) Provisional application No. 61/492,331, filed on Jun. 1, 2011.

(51) Int. Cl. G01N 21/64; G01N 33/53; C12Q 1/68

(52) U.S. Cl. CPC G01N 21/6428; C12Q 1/68; G01N 33/53
USPC 435/6.1; 530/354; 435/188; 435/7.1; 435/283.1

(57) ABSTRACT

Disclosed are compositions and methods for quantitation and calibration of images in fluorescence microscopy. Also provided are tissue phantoms that contain known amount(s) of fluorophore standard(s), as well as components and diagnostic kits containing the same for use in various histological analyses. In certain embodiments, three distinct nucleic-acid based assays provide improvements over conventional TUNEL methods to facilitate precise quantitation of a variety of nucleic acids obtained from a biological sample.

### CROSS-REFERENCE TO RELATED APPLICATIONS

The present application claims priority to U.S. Provisional Patent Appl. No. 61/492,331, filed Jun. 1, 2011, the entire contents of which is specifically incorporated herein in its entirety by express reference thereto.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### BACKGROUND OF THE INVENTION

1. Field of the Invention

The present invention relates generally to the fields of optical microscopy and molecular biology. More particularly, it concerns compositions and methods for facilitating quantitative analysis of images in fluorescence/epifluorescence-based microscopic analysis, and for analyzing and directly quantitating nucleic acids including blunt and overhanging DNA ends. In certain embodiments, the invention provides tissue phantoms containing known amounts of chromo/fluorophores to serve as analytical markers for the quantitation and calibration of biological samples undergoing histological analysis. In other embodiments, the invention provides improved methods for performing dideoxy (dd) terminal deoxynucleotidyl transferase dUTP nick end labeling (ddTUNEL) analysis of histological samples, which find particular utility in monitoring and characterizing various types of cell death.

2. Description of Related Art

Histological staining of paraformaldehyde fixed tissue sections, for diagnosis and research, has been used for more than a century. Historically, tissue sections prepared after fixation in 4% formaldehyde were found to have a better appearance and superior staining qualities when compared with the usual alcoholic fixatives that were widely used at the time. Pathologist Karl Weigert first used paraformaldehyde fixed tissue sections in 1893 and it became the fixative of choice in just a few years.

Many of the reported molecules used are fluorophores, or fluorophore-labeled antibodies as means for detection (see, e.g., The Molecular Probes® Handbook—11^{th} Edition, 2000, Invitrogen Corp., Carlsbad, Calif., USA), with the presence of the fluorophore label being detected using conventional fluorescence microscopy (Pawley, 2006). The inherent variability of fluorescence characteristics, however, has meant that such methods have, to date, only been qualitative in nature, and not quantitative. This limitation has been problematic for a number of reasons: Quantification and calibration of images in fluorescence microscopy is notoriously difficult (see e.g., Swedlow, 2007 and Wolf, 2007). Reliable quantification of fluorescence signals will permit quantitative comparison of images obtained on different microscopes, or in the same microscope employing different objectives as well as images taken days or weeks apart.

One approach to the quantification of fluorescence has been to use a fluorescence reference layer that typically contains a fluorescent dye embedded in uniform polymer film (Song et al., 1995; Talhavini et al., 1998; Zwier et al., 2007; Zwier et al., 2008; Zwier et al., 2004), such systems typically use polyvinyl alcohols as the host matrix. Such standards are made by spin-coating fluorophores, embedded within a polyvinyl alcohol matrix onto cover slides, producing a fluorophore/matrix of uniform thickness. These types of standards have greatly aided the quantification of fluorophore signals, but are not easy to prepare, are not robust and only quantify the levels of the fluorophore, not any probe to which the fluorophore may be attached. They therefore have limited utility in the calibration of biologically relevant samples (Zwier et al., 2004). Similarly, the quantitative analysis of nucleic acids using optical microscopy methods has also been limited.

Terminal deoxynucleotidyl transferase dUTP nick end labeling (TUNEL) is a common method for detecting DNA fragmentation that results from apoptotic signaling cascades. Originally described in 1992 (Gavrieli et al.), TUNEL has become one of the primary methods for detecting apoptotic programmed cell death. TUNEL detects the 3′-OH terminal ends produced by DNase type I activity by ligating these ends with labeled dUTP. Terminal deoxynucleotidyl transferase (Tdt), catalyzes the addition of dUTP to the 3′-OH ends, and dUTP has generally been labeled with digoxigenin, biotin or more recently with fluorescent labels (e.g., rhodamine, fluorescein, Texas red, as well as fluorophores of the Alexa Fluor® family [Invitrogen]).

A major problem with TUNEL is the use of dUTP as a substrate, as Tdt generates —(U)ₙ—OH 3′ polymers at each of the free DNA-OH 3′ ends; the generation of these poly-U-OH 3′ polymers is very good for signal amplification, but makes the TUNEL assay intrinsically unquantifiable. Likewise, the ability to perform quantitative analysis of nucleic acids in situ has been constrained by a lack of ligase-based assays that are suitable for the selective detection of specific markers of necrosis and/or apoptosis in tissue sections.

Reactive oxygen species produce a wide spectrum of DNA damage, including oxidative base damage and abasic (AP) sites. Many procedures are available for the quantification and detection of base damage and AP sites. However, either these procedures are laborious or the starting materials are difficult to obtain. A biotinylated aldehyde-specific reagent, ARP, has been shown to react with the aldehyde group present in AP sites, resulting in biotin-tagged AP sites in purified DNA. However, this cannot be used in a quantitative manner in either fixed cells or tissues, due to the interference of aldehydes/ketones present in other biomolecules (Kow and Dare, 2000).

### BRIEF SUMMARY OF THE INVENTION

The present invention overcomes these and other inherent limitations in the prior art by providing useful, non-obvious, and novel compositions including tissue analogs and histological phantoms, as well as methods for their use in the quantification and calibration of images in fluorescence microscopy. Using the known temperature phase-transition of gelatin-based solutions, the inventors have developed novel and non-obvious tissue analogs and phantoms that include a labeling medium making them suitable for reference standards in fluorescence microscopic analysis. The inventors have shown that by exploiting the inherent characteristics of gelatin (which has an intrinsically low absorbance in the ultraviolet [uv] spectral region), this protein can be effectively used as a labeling medium.

The present invention advantageously improves conventional compositions and methodologies for fluorescence microscopy, and provides a variety of quantization standards, and particularly those employing gelatin as the medium. In a variety of embodiments, including those described herein, gelatin has been directly, and covalently labeled with a variety of compounds including, for example, isothiocyanates N-hydroxysuccinimide ester and sulfonyl chloride fluorophore derivatives or any conventional commercially-available amine labeling fluorophores such as the more than 185 that are available commercially from Molecular Probes, or to the carboxylate groups also commercially available. The concentration of these probes has been determined using absorption spectroscopy.

This novel methodology described herein permits, for the first time, preparation of tissue phantoms containing known amounts of one or more compounds (such as without limitation: one or more fluorophores; one or more nucleic acids such as DNA or RNA; one or more peptides, polypeptides or proteins; or one or more oligonucleotide standards). The basis of these phantoms is the ability of gelatin to act as a matrix for the conjugation of fluorophores, either as a free flowing liquid or as a gelatinous solid depending on temperature.

In first illustrative embodiments, the compositions and methods disclosed herein have been used to measure the concentration of a doubled stranded DNA (including, for example salmon sperm DNA) that has been immobilized within gelatin, using ultraviolet spectroscopy. In illustrative embodiments, the detection of nucleic acids, including DNA and RNA is possible over a broad range of concentrations. In particular, the methods have been shown to be useful in detecting from about 1 to about 50 μg/mL.

The average extinction coefficient at 260 nm for double-stranded DNA is 0.020 (μg/mL)⁻¹ cm⁻¹, for single-stranded DNA and for RNA it is 0.027 (μg/mL)⁻¹ cm⁻¹. Typically, one can therefore unambiguously measure the concentration of DNA and RNA, within a 10% gelatin suspension, from 1 to 50 μg/mL for double-stranded DNA, ssDNA and RNA (an absorbance range of between 0.02 and 1 (dsDNA) and 1.35 (ssDNA or RNA) (Tataurov et al., 2008).

Similarly, in related illustrative embodiments, polypeptides and proteins (including, for example, cytochrome c), have been covalently attached to gelatin and their concentration determined by absorbance spectroscopy. Using the disclosed methods, any soluble protein, including antibodies, may be dissolved in the gelatin and covalently crosslinked to it using paraformaldehyde. In exemplary embodiments, both cytochrome c and Cy3-labeled Streptavidin have been successfully embedded in this manner.

In one embodiment, the invention provides a tissue phantom that generally includes gelatin that has been operably attached to at least a first detection moiety. Preferably, the gelatin is a mammalian skin gelatin, such as porcine skin gelatin Type A. The gelatin, preferably present in a concentration of about 7.5% to about 20%, and preferably about 15%, is linked, and preferably covalently-linked, to the detection moiety using one or more crosslinking agents, such as an amine-reactive crosslinking agents to attach one or more calibration standards thereto. In certain embodiments, the calibration standard may include one or more chromophoric dyes, one or more fluorophoric dyes, one or more oligonucleotides, one or more proteins, one or more peptides, one or more enzymes, one or more antibodies or antigen binding fragments, or any combination thereof. In an illustrative embodiment, the amine-reactive crosslinking agent is suberic acid bis(N-hydrosuccinimide ester, or a derivative or analog thereof.

In certain embodiments, the first peptide, the first protein, the first enzyme, or the first antibody may be indirectly or directly crosslinked to all or a portion of the gelatin by fixation with one or more suitable fixation reagents, including, without limitation, paraformaldehyde or the like. Preferably, the tissue phantoms of the present invention include one or more calibration standards that are adapted and configured for use in one or more suitable detection instruments, including without limitation, UV spectroscopy, or visible, fluorescence, and/or epifluorescence microscopy, or any combination thereof. In illustrative embodiments, the tissue phantoms of the present invention are preferably adapted and configured for use on a microscope slides, or in one or more wells of a multi-well assay plate, a cell culture well, a microtiter plate, or any other article of manufacture that can be utilized in one or more of the diagnostic assay methods described herein. In illustrative embodiments, (see e.g., FIG. 8), the invention concerns one or more tissue phantoms prepared on microscope slides that include one or more distinct tissue phantoms each of which contains a different, known quantity of the first calibration standard, and in some cases, will also further include a second and/or a third distinct detection moiety, or a second and/or a third distinct calibration standard that can be used to prepare a standard calibration curve which can then be used to assess the presence of, and/or to quantitate the amount of one or more selected compounds of interest within a specimen, a sample, a tissue, a cell, or any combination thereof.

The invention thus also includes an article of manufacture, such as a cuvette, a cell culture plate, a microscope slide, a microtiter dish, or a multi-well assay plate, or such like device, that includes one or more of the tissue phantoms disclosed herein. Such devices are preferably stable for extended periods of time following manufacture, such that they may have a prolonged shelf life, to permit tests to be performed some time distant from when the tissue phantom standards were prepared. For example, various concentrations of one or more known detection reagents may be prepared in one or more distinct tissue phantom compositions, and one or more of such distinct tissue phantoms can be included in a commercial form (for example, a pre-prepared microscope slide (see FIG. 7 and FIG. 8), to which a technician can then add a particular sample of interest, and the pre-prepared tissue phantoms can be used to calculate the concentration of one or more selected compounds within the sample of interest.

Exemplary devices include pluralities of distinct tissue phantoms, each of which includes a distinct, known amount of at least one calibration standard, that has been adapted and configured to facilitate the generation of a standard curve to quantitate the one or more selected compounds of interest within the specimen, sample, tissue, cell, or combination thereof that has been applied to the microscope slide and subsequently analyzed using a device such as a fluorescence or an epifluorescence microscope capable of detecting, visualizing, and/or imaging the assayed sample and the calibration standard-facilitating tissue phantom(s) present on the slide.

The present invention also includes a method of modifying a terminal deoxynucleotidyl transferase nick end labeling assay that generally involves substituting a 3′ dideoxy UTP substrate for the conventional dUTP substrate under conditions effective to facilitate detection of the reaction products resulting therefrom. Similarly, the invention also provides a method of quantitating —OH 3′ ends in a nucleic acid molecule, comprising using a ddTUNEL assay in which a 3′ ddUTP substrate is present.

The invention in further aspects provides a method of quantitating —PO₄ 3′ ends in a nucleic acid molecule. The method, as detailed herein, generally involves using an enzyme such as using calf intestinal alkaline phosphatase to convert the —PO₄ 3′ ends of a nucleic acid molecule to —OH 3′ ends, and then assaying the converted —OH 3′ ends using the ddTUNEL-based assay described herein.

The invention further provides a method of oxidizing or acetylating at least a first nucleobase of a polynucleotide molecule. This method, also detailed herein, generally involves using an enzyme such as the formamidopyrimidine DNA glycosylase (Fpg) from E. coli to convert the resulting nucleic acid molecules into ddTUNEL-positive substrates that can then be in turn, quantitated by the ddTUNEL assay described herein. In such assays, the resulting oxo-species can also further optionally be treated by borohydride reduction or by derivatization with dinitrophenyl hydrazine.

The invention also provides a method of visualizing reactive oxygen species damage within a biological cell, wherein one or more DNP-H-derivatized oxo-species are localized in the cell by detecting the presence of a labeled anti-DNP antibody, and methods for interrogating cell death within one or more cells present in a biological sample, comprising performing one or more ddTUNEL, CIAP-ddTUNEL, Fpg-ddTUNEL assays as described in detail herein, using one or more of the signal-calibrated tissue phantoms described herein, under conditions effective to monitor the level of apoptosis in one or more such cells. Such method typically involves monitoring via epifluorescence microscopy or such like as described in detail herein. In illustrative embodiments, the epifluorescence microscopy is adapted and configured with one or more optical filter blocks that include: a) a DAPI channel, b) an FITC channel, c) a Texas Red channel, or any combination thereof, such that the DAPI channel is adapted and configured for the detection of a biological probe that comprises 4′,6-diamidino-2-phenylindole; the FITC channel is adapted and configured for the detection of a biological probe that comprises an amine-reactive fluorescein derivative; and/or the Texas Red channel is adapted and configured for the detection of a biological probe that comprises sulforhodamine 101 acid chloride or a derivative or analog thereof, and the amine-reactive fluorescein derivative is a compound such as fluorescein isothiocyanate, Alex488, or DyLight488, or any combination thereof.

### BRIEF DESCRIPTION OF THE DRAWINGS

For promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates. The following drawings form part of the present specification and are included to demonstrate certain aspects of the present invention. The invention may be better understood by reference to the following description taken in conjunction with the accompanying drawings, in which like reference numerals identify like elements, and in which:

FIG. 1A, FIG. 1B, FIG. 1C, FIG. 1D and FIG. 1E show the general methodology for preparing tissue phantoms that allow the calibration of fluorescence signals in fluorescence microscopy. FIG. 1A: 3% gelatin is conjugated to amine reactive dyes, such as fluorescein isothiocyanate (FITC) or sulforhodamine 101 acid chloride (Texas Red). The excess dye is removed by ethanol precipitation and the conjugated gelatin is washed in cold water. The gelatin is then rehydrated in warm water to 15% and a concentration series is generated and dispensed into the wells of a 96-well plate. FIG. 1B: After spectroscopic determination of the conjugate concentration, warmed aliquots are dispensed into a mold, FIG. 1C. Upon paraformaldehyde (PFA) fixation, the plasticized tissue phantoms are removed from the mold, FIG. 1D, and washed in buffer, then treated as pathological specimens undergoing dehydration, waxing, slicing and mounting on slides (FIG. 1E);

FIG. 2A and FIG. 2B show the design of the blunt ended (FIG. 2A) and overhanging (FIG. 2B) specific DNA probes and standards. Standards are conjugated to gelatin using suberic acid bis-succinimide, a homo-bifunctional cross-linking reagent with amine reactivity, and their concentration measured using uv spectroscopy at 260-280 nm. The probes are labeled (either with Texas Red or with Alexa Fluor® 405), and each of the oligonucleotides has a restriction endonuclease site that allows non-specific binding to be quantified. The Texas Red-labeled blunt-ended probe was manufactured in its final form by Oligo Factory (Houston, Tex., USA). The overhanging oligonucleotide probe was prepared from an N-hydroxysuccinimide (NHS)-carboxy-dT oligonucleotide manufactured by Oligo Factory, to which the cadaverine form of the Alexa Fluor® 405 dye was coupled (Invitrogen Corp., Cat. No. A-30675), using the manufacturer's instructions;

FIG. 3A and FIG. 3B show how a standard solution of 2,4-dinitrophenol (DNP)-conjugated gelatin may be used as an internal standard for the quantification of a second chromophore/fluorophore. A Cy3-streptavidin serial dilution was prepared using 15% gelatin that was conjugated with 84 μM DNP. The stock concentration of a commercially obtained Cy3-streptavidin conjugate (ZyMed. Corp., San Francisco, Calif., USA) was determined spectrophotometrically as 81 μM streptavidin and 44 μM Cy3 based on the extinction coefficients of the streptavidin tetramer (ε₂₈₀=165,304 M⁻¹ cm⁻¹) and Cy3 (ε₅₅₉=150,000 M⁻¹ cm⁻¹). Cy3 streptavidin was dissolved in an equal volume of 30% gelatin, and then diluted it in a 1:1 ratio with DNP-conjugated gelatin (168 μM DNP). This generated a stock Cy3-streptavidin solution that contained 15% gelatin, 84 μM DNP, 20 μM streptavidin, and 11 μM Cy3. This stock underwent a serial dilution from 5.5 μM to 150 nM Cy3 in 84 μM DNP-gelatin, and the optical spectra of 300-μL aliquots (1-cm path length) were recorded. Casts were prepared from 200-μL aliquots taken from each well, and the absorbance of the approximately 3-mm phantom blocks was taken before being mounted on slides. The absorbance spectra was taken of the dilutants in a 96-well plate (FIG. 3A) and in the PFA-treated casts (FIG. 3B). The use of DNP allows the path length of the two absorbance series to be determined, so that the DNP signal at 360-nm signal is an internal standard for the 560-nm Cy3-streptavidin signals;

FIG. 4A, FIG. 4B, FIG. 4C, FIG. 4D, FIG. 4E, FIG. 4F, FIG. 4G, FIG. 4H, FIG. 4I, and FIG. 4J show images obtained from sixty different phantoms, showing the dynamic range and sample homogeneity. The first three series, FIG. 4A, FIG. 4B, and FIG. 4C, show the signals generated in 6 μM phantoms where a fluorophore is directly conjugated to gelatin with Texas Red, FITC, and Rhodamine-B. FIG. 4D shows the signals generated in Cy3-streptavidin, conjugated to gelatin by PFA during the fixing process. FIG. 4E and FIG. 4F show the use of a primary/secondary antibody pairing for the visualization of DNP and cytochrome c, respectively. Anti-DNP antibody (Sigma Aldrich Chemical Co., St. Louis, Mo., USA) produced in rabbit was used as the primary and imaged using Alexa Fluor® 594 goat anti-rabbit immunoglobulin (IgG) and additionally, mouse anti-cytochrome c antibody (Abcam®). Murine IgG was used as the primary antibody, and it was imaged using Alexa Fluor® 488-labeled goat anti-mouse IgG. FIG. 4G and FIG. 4H show the signals generated by in situ ligation of fluorescently labeled oligonucleotide probes to a conjugated oligonucleotide standard. The final two series show the fluorescence of salmon sperm DNA, immobilized within fixed gelatin, following treatment with using 1 μM YO-PRO-1® in Fluoromount-G® (SouthernBiotech) and DAPI (Slowfade Gold® with DAPI, Invitrogen), (FIG. 4I and FIG. 4J, respectively);

FIG. 5A, FIG. 5B, FIG. 5C, and FIG. 5D show the relationship between absorbance at 592 nM and the fluorescence of 300-μL samples of Texas Red conjugated 15% gelatin, measured in a 96-well plate, showing the non-linearity of the relationship at concentrations>15 μM FIG. 5A; the average fluorescence, n=4, of seven different concentrations of Texas Red phantoms cut to a thickness of 5 μm, and measured at a magnification of 40× for 1.5 sec. FIG. 5B; the average fluorescence, n=4, of the in situ-ligated and overhanging probes to their respective standards. The Texas Red-labeled blunt-ended probe ligated to its oligonucleotide standard had essentially the same fluorescent properties as the Texas Red-conjugated gelatin standards (FIG. 5C). The four panels in FIG. 5D show that the images of Texas Red bound to either gelatin or to gelatin via the oligonucleotide pairings have the same fluorescence signal (I and II). The stability of the dehydrated, waxed samples is high, with no loss of signal in a sample stored for three months (III). The last image (IV) shows the signal levels of a ligated, blunt-ended standard, at 15 μM, following incubation with the restriction endonuclease, EcoRI. The lack of signal indicates that there is little or no, non-specific binding of the blunt-ended probe to the gelatin matrix;

FIG. 6A and FIG. 6B show the optical spectra of conjugated cytochrome c and fluorescence of cytochrome c phantoms. FIG. 6A shows the reduced minus oxidized difference spectra of cytochrome c conjugated to 10% gelatin and indicates that there were no spectral perturbations of the heme. FIG. 6B shows that it was possible to quantify the levels of cytochrome c in phantoms using a primary/secondary antibody pair, a mouse anti-cytochrome c monoclonal antibody, and an Alexa Fluor® 488-labeled goat anti-mouse secondary antibody;

FIG. 7A, FIG. 7B, FIG. 7C, and FIG. 7D show the effect of the chemotherapeutic agent irinotecan (i.e., (S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxy late), on levels of blunt and overhanging breaks in U87 cells. Shown is the damage caused to U87 cells by irinotecan measured using the fluorescence signal generated by Texas Red-labeled blunt ends (red), Alexa Fluor® 405-labeled overhangs (blue) and YO-PRO-18-labeled DNA (green). FIG. 7A shows that control cells had few DNA breaks, (presented graphically in FIG. 7C, which shows the total number of molecules along the y-axis). Treatment with irinotecan increased the number of blunt-ended breaks by more than 50%, and the number of overhanging breaks by almost 3-fold. The two color-bars on the upper left and right show the approximate color levels that resulted from a 6 μm slice of DNA (mM) and Texas Red (μM);

FIG. 8 illustrates the use of Fpg-ddTUNEL and DNPH to discriminate between modified bases and AP sites. Shown is a representative damaged section of double stranded DNA (exemplified in SEQ ID NO:8 and SEQ ID NO:9) with an oxidized guanine (G=O) and an AP site (R=O), (Scheme A). The sample can be incubated with DNP-H, (Scheme B), to convert the AP site into a hydrazone, which is not a substrate for Fpg (exemplified in SEQ ID NO:17 and SEQ ID NO:18). The samples are treated with Fpg (exemplified in SEQ ID NO:11, SEQ ID NO:12, SEQ ID NO:13, SEQ ID NO:14, SEQ ID NO:19, SEQ ID NO:20, SEQ ID NO:21, and SEQ ID NO:22), followed by CIAP, to generate 3′OH ends and then with ddTUNEL (exemplified in SEQ ID NO:15, SEQ ID NO:16, SEQ ID NO:23, and SEQ ID NO:24). In samples not treated with DNPH (Scheme A), both the 8-OG and AP sites are labeled with ddUTP. For the derivatized sample (Scheme B), only 8-OG was labeled by ddTUNEL. However, the presence of the ribose-hydrazone can be independently-labeled using an anti-DNP antibody;

FIG. 9A, FIG. 9B, and FIG. 9C show how FITC-labeled gelatin tissue phantoms could be used to calibrate fluorescence signals for the ddTUNEL reaction. The images shown in FIG. 9A are of different labeled FITC-phantoms, with FIG. 9B showing the average fluorescence (n=3), of those standards, and those from two other slides (Mag: 100×; accumulation time 100 msec). FIG. 9C shows that the fluorescence of the 11.5-μM standard was proportional to exposure time; thus, from the averaging of signals from know fluorophore phantoms it is possible to construct a standard curve, which may then be used to signals obtained by this same fluorophore for the measurement of an unknown level of a biological molecule; i.e., FIG. 10A to FIG. 10L.

FIG. 10A, FIG. 10B, FIG. 10C, FIG. 10D, FIG. 10E, FIG. 10F, FIG. 10G, FIG. 10H, FIG. 10I, FIG. 10J, FIG. 10K, and FIG. 10L show the validation of the ddTUNEL and Fpg-ddTUNEL assays in rat mammary gland. This figure illustrates the validation of the ddTUNEL (biotin-ddUTP/FITC-avidin) and Fpg-ddTUNEL (PromoFluor-594 ddUTP; PromoKine/PromoCell Gmbh, Heidelberg, GERMANY) assays rat mammary gland on Day 1 and Day 7 of involution. In FIG. 10A, FIG. 10B, and FIG. 10C, the presence of ddTUNEL/Fpg-ddTUNEL-positive cells, and the formation of apoptotic bodies are shown (at three different magnifications) in breast on Day 1. The same labeling of tissue on Day 7 of involution is shown in FIG. 10E, FIG. 10F, and FIG. 10G. The solid arrows in panel FIG. 10B indicate two cells that are in the early stages of apoptosis and the cell shown by the dotted arrow is in a later stage of apoptosis. The apoptotic bodies contain ddTUNEL positive DNA that is associated with histone γ-H2A.X (see FIG. 10D and FIG. 10H). The color of the ddTUNEL probe was switched to red (PromoFluor-594 ddUTP), to demonstrate that the florescence resulted from the appropriate probes, and was not due to cytoplasmic lipofuscin pigment. In FIG. 10I and FIG. 10J, CD3ε-positive immune cells were shown to be near apoptotic cells and apoptotic bodies, and to contain apoptotic DNA that came from these cells. Finally, FIG. 10K and FIG. 10L show the presence of ddTUNEL/Fpg-ddTUNEL DNA in apoptotic bodies on Days 1 and 7 that was not labeled by DNP-H, which lack AP sites. The ddTUNEL/Fpg-ddTUNEL apoptotic DNA was surrounded by oxidized protein, which was itself surrounded by dying cells. DNA was counterstained by DAPI where indicated;

FIG. 11A, FIG. 11B, FIG. 11C, FIG. 11D, FIG. 11E, FIG. 11F, FIG. 11G, FIG. 11H, FIG. 11I, FIG. 11J, FIG. 11K, and FIG. 11L illustrate the use of the ddTUNEL and Fpg-ddTUNEL assays to assess the DNA damage caused to U87 cells. Shown is the use of biotinylated ddUTP, in ddTUNEL (Fluram®-avidin; fluorescamine, Hoffman-LaRoche and Co.) and Fpg-ddTUNEL (Texas Red-avidin) assays of DNA damage caused to U87 cells, following incubation with reactive oxygen species (ROS)-inducing and chemotherapeutic reagents. In FIG. 11A to FIG. 11F, DNA was stained with green YO-PRO-1® and in FIG. 11G to FIG. 11L green γ-H2A.X is shown. The stressors all increased the levels of DNA damage; these levels are shown in Table 1, and discussed in the following Examples;

FIG. 12A, FIG. 12B, FIG. 12C, FIG. 12D, FIG. 12E, and FIG. 12F show the determination of oxidized and acylated DNA bases using sodium borohydride (NaBH₄) and the Fpg-assay, and the discrimination between acylated and oxidized bases using NaBH₄ reduction. In FIG. 12A, FIG. 12B, and FIG. 12C, green ddTUNEL- and red Fpg-ddTUNEL-positive DNA damage is shown in cells treated with irinotecan] i.e., (S)-4,11-diethyl-3,4,12,14-tetrahydro-4-hydroxy-3,14-dioxo1H-pyrano[3′,4′:6,7]-indolizino[1,2-b]quinolin-9-yl-[1,4′bipiperidine]-1′-carboxylate, a topo-isomerase I inhibitor]; H₂O₂ (which oxidizes DNA); and carmustine (BiCNU®, Bristol-Myers Squibb; BCNU; N,N′-bis(2-chloroethyl)-N-nitroso-urea, a DNA ethylating agent). In the lower three panels (FIG. 12D, FIG. 12E, and FIG. 12F) cells are shown from the same slides that have been treated with an ethanolic solution of NaBH₄. What was apparent was that the red Fpg-ddTUNEL-positive DNA damage signal had been altered. The signal was lowered by borohydride reduction as all the oxo-bases had been reduced and were no longer substrates for the Fpg-ddTUNEL assay. The levels of oxo-bases in the irinotecan- and carmustine-treated cells were unaltered by reduction, as would be expected given that irinotecan and carmustine do not appear to increase ROS levels, either intuitively (i.e., one would have expected no increase in ROS damage in these cells, but an increase in DNA breaks and an increase in acylated bases, respectively), or by actual measurement;

FIG. 13 shows the use of the dc/TUNEL and CIAP-ddTUNEL to measure 3′OH and 3′PO₄ DNA ends. Shown is a representative damaged section of double stranded DNA with both DNase Type I- and Type II-ends. After a round of ddTUNEL, using a green-labeled ddUTP all 3′OH-ends were labeled (1). All 3′PO₄ DNase Type II-ends are converted into ddTUNEL positive 3′OH ends using CIAP, (2) and these are labeled with red ddUTP (3), in a second round of ddTUNEL;

FIG. 14 shows the labeling of DNase type-II treated U87 cells using ddTUNEL and CIAP-ddTUNEL to measure 3′OH and 3′PO₄. U87 cells were grown on slides, fixed, permeabilized, washed and then treated with DNase II for 2 hrs. The 3′OH ends were labeled green using ddTUNEL (biotin-ddUTP/FITC-avidin). The levels of 3′OH ends in DNase II treated cells were identical to those of control cells incubated with the enzyme omitted from the DNase II buffer. After ddTUNEL half of the samples were incubated with CIAP and the other half with only CIAP buffer. The CIAP positive and negative samples were then incubated with a second ddTUNEL assay mixture (biotin-ddUTP/FITC-avidin). Only cells that were incubated with CIAP, (A), were labeled in the second round of ddTUNEL, cells in which 3′PO₄ ends were not converted in vitro into 3′OH ends, (B), were only stained in the initial ddTUNEL round;

FIG. 15A, FIG. 15B, FIG. 15C, and FIG. 15D show exemplary commercialization embodiments of labeled tissue phantoms (“slide ladders”) with fluorophoric phantom “ladder rungs” each containing different concentrations of the standard (FIG. 15A). FIG. 15B shows another exemplary commercialization of labeled tissue phantoms (slide “polka dots”) with each of the standard “dots” containing different concentrations of an IgG antibody. FIG. 15C shows another exemplary commercialization of labeled tissue phantoms (slide “polka dots”) with fluorophoric phantom “dots,” each containing different concentrations of the standard. FIG. 15D shows an illustrative embodiment of the invention in which tissue phantom “flowers” were prepared as described herein, and then placed on a standard microscope slide to serve as a standard for quantitating the contents of a sample placed alongside the tissue phantom and imaged by fluorescence microscopy. The final fluorophoric gelatin “petals” (each containing a different, known, concentration of a known standard fluorophore) were arranged around a central “core” path-length standard (e.g., DNP), which permits determination of the exact thickness of the imaged sample;

FIG. 16A and FIG. 16B show a comparison of TUNEL with ddTUNEL. FIG. 16A shows the use of 2′ dUTP in the TUNEL assay leads to the unquantifiable polymetric labeling of a single 3′OH present on demonic DNA. Each labeled dUTP added to a 3′OH end acts as a substrate for a subsequent dUTP. In contrast, using 2′,3′ ddUTP in the ddTUNEL assay ensures that one and only one labeled ddU is added to each 3′OH DNA end. The structures of deoxyribose and dideoxyribose are shown in the appropriate panel, and the arrow indicates the presence of 3′H in ddUTP;

FIG. 17A and FIG. 17B show changes in mitochondrial membrane potential and of ROS generation induced by ethylmercury in normal human astrocytes. Changes in mitotracker and ROS-induced DCF levels caused by incubation with increasing concentrations of Thimerosal (FIG. 17A) and the time course of incubation with 14.4 μM thimerosal (FIG. 17B) with respect to control cultures. Cells were imaged in the centerfield of three independent wells, consisting of an average of 44 cells per field, with a standard deviation of 16.5;

FIG. 18A, FIG. 18B, FIG. 18C, and FIG. 18D show co-localization of Mitotracker (ΔΨ) and DCF (peroxide) fluorescence in Normal Human Astrocytes: Thimerosal induces oxidative stress at the mitochondrial level. High-resolution images of control NHAs and NHAs treated for 60 min with 14.4 μM Thimerosal. FIG. 18A: Mitotracker (red), ROS induced DCF (green), and nuclear Hoechst staining (blue) of NHAs at 60× in the absence (left) and presence (right) of 14.4 μM Thimerosal. FIG. 18B: Images of control and treated cells obtained at 150× magnification. An orange-colored ‘horseshoe-shaped’ signal in the control cell consists of a network of mitochondria which is mirrored in the ROS induced DCF image. The same is demonstrated in the treated cells by a ‘lightening bolt’-shaped mitochondrial network. FIG. 18C: Square outlines of the cells from FIG. 18B highlighting individual Mitotracker and ROS images, and their overlaid images. FIG. 18D: Intensity profile of MT, DCF and Hoechst along the two diagonal lines in FIG. 18B (with the MT signal 4× in the Thimerosal-treated image). Red: MT signal, blue: Hoechst signal, green: ROS generated DCF, black: fit to the ROS signal, based on the amplitudinal changes of MT and Hoechst. The two simulations indicate that four times the amount of DCF is generated by mitochondria in the ethylmercury treated cells, but background cytosolic rates of generation are the same;

FIG. 19A, FIG. 19B, and FIG. 19C show co-localization of Mitotracker and carbonyls in Normal Human Astrocytes: Thimerosal induces oxidative damage at the mitochondrial level; Control and 14.4 μM Thimerosal treated cells prepared using MT (red) and Hoechst (blue), with FITC-Avidin/Biotin-Hydrazide carbonyl labeling (green). FIG. 19A: A large ethylmercury treated cell showing an increase in green ROS damaged cell contents as a function of distance from the nucleus. FIG. 19B: Two boxes from FIG. 19A highlighting the correlation between MT (red) and carbonyl (green) signals. FIG. 19C: The two vertical lines in FIG. 19A indicate the position of fluorophore intensity profile interrogation. Red: MT, green: carbonyls, blue: Hoechst, black: a simulation of the levels of ROS damage generated by combining fractions of the MT and Hoechst signals. In both samples the simulation is a poor match for the actual ROS-induced signal, with cross-correlations of ROS vs. simulation giving R2 values of only 0.68 and of 0.86, respectively;

FIG. 20A, FIG. 20B, and FIG. 20C show mitochondrial superoxide production correlates with hydroxyl radical generation and mtDNA damage in Normal Human Astrocytes: Thimerosal potentiates Fenton/Haber-Weiss chemistry in the mitochondrial matrix. Control NHAs and NHAs incubated for 1 hr with 14.4 μM Thimerosal. Production of ROS measured with the mitochondrial superoxide probe MitoSox™ (red), and measurement of HO. via hydroxyphenyl fluorescein (HPF) (green) in FIG. 20A, 3′OH DNA ends with ddTUNEL (green) in FIG. 20B, and blunt ended DNA breaks (green) in FIG. 20C;

FIG. 21 shows a summary of observed changes in NHA following incubation with ethylmercury. Bar plot showing the summarized changes observed in NHA following a one hour exposure to 14.4 μM Thimerosal, with respect to untreated controls. Each value is expressed as mean±SD of five fields measured in the center field of triplicate experiments. Each of the treated values is statistically different from the controls at p<0.01. The increase in HO. is statistically different from the increase in H₂O₂ (H₂DCF-AM) at p<0.01. Statistical analyses were performed using one-way analysis of variance (ANOVA) with the Holm-Bonferroni post hoc test. The test was performed only when the results of ANOVA were p<0.05, using Daniel's XL Toolbox, a free, open source add-in for Microsoft Excel; and

FIG. 22A, FIG. 22B, and FIG. 22C show proposed mechanism for the toxicity of organomercury. FIG. 22A: As a lipophilic cation, ethylmercury will become concentrated inside astrocytes, following the plasma membrane potential of 45 mV, by a factor of 5.6-fold, and cytosolic ethylmercury will partition into the mitochondria by a factor of 1,000 fold, its accumulation driven by the approximate 180 mV mitochondrial membrane potential. FIG. 22B: Inside the mitochondria, ethylmercury reacts and caps thiols/selenols, including the cysteine residues of iron-sulfur centers. The formation of ethylmercuricthiol adducts causes not only enzyme inhibition, but also increases the levels of free iron inside the mitochondria. FIG. 22C: The release of iron catalyzes Fenton/Haber-Weiss chemistry leading to the formation of the highly oxidizing HO. HO. has multiple targets, including sensors of the permeability transition complex and also mtDNA. High levels of HO. cause Mitoposis, leading to cytochrome c release from the mitochondria and the initiation of apoptosis.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

Fluorescence/Epifluorescence Microscopy

Fluorescence/epifluorescence microscopy is a method widely used in life sciences to image biological processes in living and fixed cells or in fixed tissues (Waters, 2009; Storrie et al., 2008; Suzuki et al., 2007). Epifluorescence microscopes are similar to conventional reflecting optical microscopes in that both types illuminate the sample, and produce a magnified image of the sample. Whereas conventional reflecting optical microscopes use the scattered illumination light to form an image, epifluorescence microscopes use the emitted fluorescent light to form the image. Epifluorescence-based microscopy requires higher intensity excitation (illumination) light than in conventional microscopy, with the higher intensity excitation light needed to excite one or more fluorescent molecules present in the sample, causing them to emit fluorescent light. Because the excitation light has a higher energy (i.e., shorter wavelength) than the emitted light, epifluorescence microscopes can use the emitted light to produce a magnified image of the sample. A particular advantage of epifluorescence-based microscopy is that the sample may be prepared such that one or more fluorescent molecules may be preferentially attached to one or more biological structure(s), molecule(s), or cell(s) of interest within the sample thereby facilitating their imaging.

Quantification and calibration of images in fluorescence microscopy is notoriously difficult (Swedlow, 2007; Wolf, 2007). Reliable quantification of fluorescent signals will permit quantitative comparison of images obtained on different microscopes, or on the same microscope employing different objectives as well as images taken days or weeks apart. One approach to the quantification of fluorescence has been to use a fluorescent reference layer that typically contains a fluorescent dye embedded in uniform polymer film (Song et al., 1995; Talhavini et al., 1988; Zwier et al., 2007; Zwier et al., 2008; Didenko and Baskin, 2006). Such systems typically use polyvinyl alcohols as the host matrix. Such standards are made by spin-coating fluorophores, embedded within a polyvinyl alcohol matrix onto cover slides, producing a fluorophore/matrix of uniform thickness. These types of standards have greatly aided the quantification of fluorophore signals, but are not easy to prepare, are not robust and quantify the levels of only the fluorophore, not any probe to which the fluorophore may be attached. They therefore have limited utility in the calibration of biologically relevant samples (Didenko and Baskin, 2006).

TUNEL and ddTUNEL Assays for DNase Type II Activity

DNase type II enzymes are associated with the endoplasmic reticulum/lysosome and their activity is often indicative of either necrosis or of caspase-independent calpain/serpin driven apoptosis (Counis and Torriglia, 2006; Reme et al., 1998; Torriglia and Lepretre, 2009). The —PO₄ 3′-ends that result from DNase Type II activity are not substrates for the Tdt dependent TUNEL assay. However, the —PO₄ 3′-ends in a tissue sample can be easily converted to —OH 3′ ends, suitable for TUNEL labeling, by incubation with a phosphatases. Calf intestinal alkaline phosphatase (CIAP) has been used to convert the —PO₄ 3′-ends generated by DNase Type II activity into TUNEL-positive —OH 3′-ends (Lorenz and Schroder, 2001).

FPC-ddTUNEL assay

E. coli formamidopyrimidine-DNA glycosylase (Fpg or MutM) is a DNA repair enzyme that excises damaged DNA bases from double-stranded DNA leaving behind a gap flanked by 3′- and 5′-phosphate ends (Gill et al., 1996; O'Connor and Laval, 1989; Ropolo et al., 2006; Speit et al., 2004; Wu et al., 2002; Ying-Hui et al., 2002; Xu et al., 2001). Incubation of DNA with CIAP after incubation with Fpg generates one ddTUNEL positive —OH 3′-end for each acylated/oxidized base substrate. Therefore, by combining Fpg/CIAP with ddTUNEL one may quantify the total levels of oxidized/modified Fpg-positive base modifications in a sample.

Using a combination of ddTUNEL and Fpg/CIAP TUNEL, with internal calibration using fluorescently-labeled tissue phantoms, one can observe, for the first time, the relative levels of different types of DNA damage in fixed cells or tissue samples in a quantitative manner. Quantification of fluorescence signals in microscopic samples allows direct comparisons of DNA damage in different cell types, DNA damage caused by different stimuli, or between samples collected and analyzed at different times, or under different laboratory conditions.

### EXEMPLARY DEFINITIONS

The term “for example” or “e.g.,” as used herein, is used merely by way of example, without limitation intended, and should not be construed as referring only those items explicitly enumerated in the specification. In accordance with long standing patent law convention, the words “a” and “an” when used in this application, including the claims, denote “one or more.”

### EXAMPLES

The following examples are included to demonstrate illustrative embodiments of the invention. It should be appreciated by those of ordinary skill in the art that the techniques disclosed in the examples that follow represent techniques discovered by the inventor to function well in the practice of the invention, and thus can be considered to constitute preferred modes for its practice. However, those of ordinary skill in the art should, in light of the present disclosure, appreciate that many changes can be made in the specific embodiments that are disclosed and still obtain a like or similar result without departing from the spirit and scope of the invention.

### Example 1

### Preparation of FITC-Labeled Gelatin Phantoms; Identification and Calibration of Images in Fluorescence Microscopy

Fluorescently-labeled, oligonucleotide probes have been developed, which can be used, inter alia, to study programmed cell death, or for visualizing the presence of different types of DNA breaks (see, e.g., Didenko et al., 2006; Didenko et al., 2004; Baskin et al., 2003; Didenko et al., 2003; Didenko et al., 2002; Didenko, Ngo and Baskin, 2002; Didenko et al., 1999) in a biological sample. This methodology was facilitated by the development of tissue “phantoms” that contain known amounts of: 1) one or more chromophores or fluorophores; 2) one or more nucleic acids (e.g., a DNA, an RNA, or any combination thereof); 3) one or more peptides, proteins, polypeptides, enzymes, and/or antibodies (either labeled or unlabeled), or any combination thereof; 4) one or more oligonucleotide standards (either DNA, RNA, or any combination thereof); or 5) any combination of one or more of the compounds or molecules in 1) through 4).

This example describes the preparation and use of such tissue phantoms, and details the methodology useful in preparing and utilizing a wide range of molecular standards in a variety of conventional fluorescence-based microscopy protocols. In particular, a key feature of these tissue phantoms is the creation of a gelatin matrix to which one or more known fluorophores can be covalently conjugated (i.e., linked), and which may exist either as a free-flowing liquid or as a gelatinous solid depending on incubation temperature (e.g., ≧40° C. and ≦4° C., respectively), from which calibration standards may be developed to permit quantitation of one or more selected compounds of interest within an imaged specimen, sample, tissue, cell, or such like.

Materials and Methods

All data presented in this example utilized porcine skin gelatin (Type A) (Sigma-Aldrich, Cat. No. 9000-70-81), FITC (Sigma-Aldrich, Cat. No. F7250), Cytochrome c, rhodamine B isothiocyanates (Sigma-Aldrich, Cat. No. R1755), Texas Red (sulforhodamine 101 acid chloride, Sigma-Aldrich, Cat. No. S3388), and suberic acid bis-succinimide (Sigma-Aldrich, Cat. No. S1885). 6-(2,4-dinitrophenyl)aminohexanoic acid, succinimidyl ester (DNP-DHS) was obtained from Invitrogen Corp. (Cat. No. D2248; Carlsbad, Calif., USA) and all reagents were used as supplied. PELCO® 20-cavity embedding silicone molds were purchased from Ted Pella, Inc. (Redding, Calif., USA). Oligonucleotide standards were purchased from Integrated DNA Technologies (Coralville, Iowa, USA). The Texas Red-labeled, blunt-ended probe was manufactured in its final form by Oligo Factory, and the labeled overhanging oligonucleotide probe was prepared from an NHS-Carboxy-dT oligonucleotide manufactured by Oligo Factory to which the cadaverine form of Alexa Fluor® 405 dye (Invitrogen Corp., Cat. No. A-30675) was coupled.

General Methodology for Phantom Preparation

One aspect of the present invention, the preparation of tissue phantoms, makes use of the well-known phase-change that hydrated gelatin undergoes upon heating and cooling. The most common use of gelatin is in the production of table jelly or jelled snacks such as Jell-O (Kraft Foods, Northfield, Ill., USA). Gelatin is only sparingly soluble in cold water; however dry gelatin swells or hydrates when stirred into water at <34% gelatin, and upon warming to >40° C. melts to give a uniform solution. Gelatin at 15% has a temperature transition that allows it to be converted from a semi-solid at 4° C. to a free and pipettable liquid at 45° C. Gelatin is essentially optically transparent in the UV region, having only small amounts of the UV chromophores, tyrosine, tryptophan and histidine. This property permits optical spectroscopy on the phantoms before fixing, to examine the concentration of chromophores like DNA or crosslinked proteins or peptides. Additionally, gelatin can be easily purified by precipitation in cold ethanol.

In this study gelatin has been covalently labeled with amine reactive cross-linking reagents attached to chromophoric or fluorophoric dyes, oligonucleotides and proteins. Excess dye/oligo/protein was separated from the conjugated gelatin by washing in ethanol, which precipitated gelatin, and then washing in cold water. The amount of probe bonded to gelatin was determined by absorbance spectroscopy.

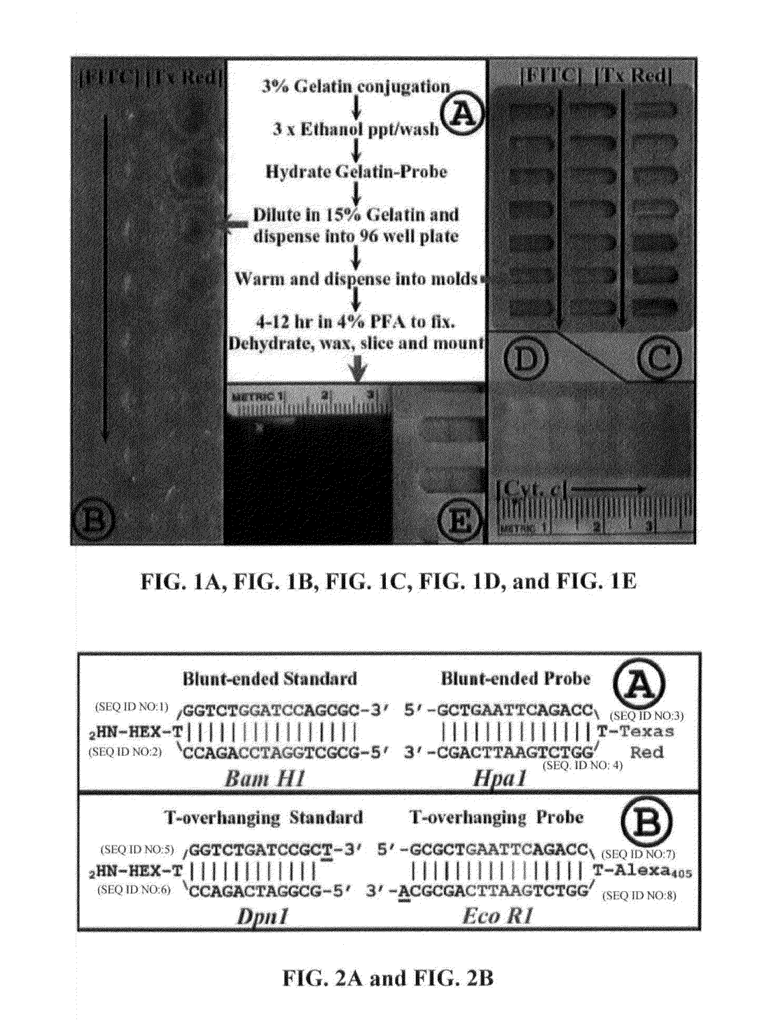

Standard solutions of conjugated gelatin were then melted at 45° C. and cast into silicone molds. They were cooled to about 4° C., fixed in 4% paraformaldehyde overnight, and then subjected to the same histological procedures as tissue samples. After slicing, de-waxing and hydration the labeled tissue phantoms were used to prepare standard curves of fluorescence signal vs. probe concentration. Essentially, ethanol-precipitated gelatin is weighed, then hydrated (using warm water and physical mixing) until it forms a completely mixed, free-flowing liquid. This liquid is then transferred into a silicone mold using a pipette, and the mold is placed in a refrigerator until cool (in a manner analogous to the way one makes edible gelatin snacks). Cold gelatin has a rubbery consistency (similar to that of a chilled gelatin dessert), which can be submerged in 4% PFA overnight for fixing. After fixing is completed, the phantom can be removed from the silicone mold and the resulting cast now has the properties of a soft rubber/flexible plastic. FIG. 1A, FIG. 1B, FIG. 1C, FIG. 1D and FIG. 1E show the general methodology of the process, including representative images. The following section details how specific phantoms are prepared.

Gelatin Sources:

In preliminary studies, a large number of different types of gelatin were tested, from various chemical companies and from supermarkets: gelatin from bovine skin Type B (Sigma G9382), gelatin from coldwater fish skin (Sigma-Aldrich, Cat. No. G7041), granular gelatin for laboratory use (Fisher Scientific, Inc., Cat. No. G8-500), as well as food-grade gelatins from grocers such as Whole Foods Market and the Kroger Company. Of the samples tested, porcine skin gelatin; Type A, (Sigma-Aldrich) was found to have the best properties for the preparation of phantoms, as it has a clean optical spectrum in the UV region, forms a pipettable free flowing liquid at 45° C. at 15% (wt./vol.), and is also a liquid at 30% at this temperature. This gelatin does not dissolve in ice-cold water but hydrates rapidly at 45° C. following ethanol precipitation. The other gelatins tested, although still usable, had poorer flow qualities upon heating, and/or produced less-solid gels upon refrigerating.

Washing Gelatin:

Following conjugation, the excess, un-reacted probe was separated from gelatin using cold ethanol precipitation. It was found that after pre-washing gelatin in ethanol, ethanol soluble fractions were removed, so that later ethanol precipitation steps had a higher yield (i.e., if 5% of the gelatin is soluble ethanol, this fraction will take up label, but be lost in the precipitation step; however, if it is removed prior to probe conjugation then the efficiency of the conjugation/precipitation process is improved). Gelatin was washed at room temperature in 95% ethanol, at five volumes of ethanol to one volume of gelatin. After centrifugation and drying under vacuum, the gelatin was washed with ice-cold water and after decanting, was washed once more in 100% ethanol and dried. This washing process removes low molecular weigh peptides that reduce the recoverable yield of conjugated gelatin. Washing is not essential if the probe being conjugated is of low cost (e.g., FITC), but is more important if the probes being used are expensive (e.g., for the conjugation of synthetically-prepared oligonucleotides and the like).

Gelatin stock solutions for phantom casting were typically 15%, but it was demonstrated that 10% gelatin was optimal when suberic acid bis(N-hydroxysuccinimide ester) was used as a cross-linking agent. The range of gelatin concentrations that have been used range from 7.5 to 20%. The exact, optimal, concentration of gelatin depends on the levels of and type of probe being suspended (e.g., when proteins are used) within the gelatin or being covalently bonded to it. What is important is that the mixture forms a free-flowing liquid when warm, allowing accurate dispensation, and forms a more-or-less firm gel when cooled, so that it will not dissolve when 4% PFA is added.

Preparation of Isothiocyanate-, Sulfonyl Chloride- and N-Hydroxysuccinimide-Labeled Gelatin Conjugates:

A very large selection of amine- and carboxylate-reactive chromophore/fluorophores are commercially available (e.g., there are more than 150 different fluorophores that can be used to conjugate gelatin from one supplier alone—see; e.g., “Fluorophores and Their Amine-Reactive Derivatives—Chapter 1 The Molecular Probes® Handbook,” 11th Ed.). Typically, protocols for the conjugation of isothiocyanates, sulfonyl chlorides and N-hydroxy-succinimides suggest that the probe should be dissolved in an organic solvent (such as DMF) and an organic buffer (such as 1.0% diisopropylethylamine), and then added to the protein (see, e.g., The Molecular Probes® Handbook). These protocols were used as an initial starting point, and they achieved quite low coupling efficiencies (e.g., 20% to 50%).

It was found that higher coupling efficiencies could be achieved by the addition of a few grains of the amine-reactive probe to warm gelatin. Gelatin was dissolved in 1 mL of 10 mM potassium phosphate, pH 7.0, to a concentration of 3% in a 15-mL centrifuge tube and warmed to 45° C. A small amount (≦200 μg) of the solid probe (containing a reactive isothiocyanate, a sulfonyl chloride or an N-hydroxy-succinimide) was added from the tip of a spatula, and the solution was then mixed by vortexing for a few minutes, and incubated for ˜1 hr in a 45° C. water bath. The gelatin-conjugate was then precipitated by the addition of 14 mL of ice-cold 100% ethanol. The tube was centrifuged, a 1-mL aliquot of the supernatant was removed, and the concentration of free probe determined using a spectrophotometer. The conjugated gelatin pellet was then washed twice in 15 mL of 95% ethanol and, after drying under vacuum, was re-suspended in 200 μL of hot water (≈65° C.). Tracking the levels of free probe in each of the three ethanol supernatants allowed the coupling efficiency to be determined. Coupling efficiencies of >80% were achieved using FITC, ≈75% using rhodamine B isothiocyanate or Texas Red sulfonyl chloride, and >60% using DNP-NHS.

Casting and Fixing of Standard Blocks:

The general methodology is shown in the flow chart seen in FIG. 1A. A fluorescently-labeled, 3% gelatin solution was dissolved in 19% gelatin (in a 1 to 3 ratio), at 45° C. to produce a final gelatin concentration of 15%. The warm gelatin was pipetted into Eppendorf tubes containing 15% gelatin in a heating block. After vortexing and centrifugation to remove bubbles, 300-μL aliquots were dispensed into the wells of a 96-well plate, 1 cm path length (see FIG. 1B). The accuracy of pipetting was increased by pre-warming and pre-hydrating each pipette tip in 0.1 M PBS buffer heated to 45° C. The spectrum of the plate was taken to determine the concentration of conjugate in each well; the fluorescence of the samples was also examined at this time to establish the relationship between concentration and fluorescence. After reading, the 96-well plate was reheated to 45° C. and 200-4 aliquots were transferred from the wells into silicone molds (PELCO), FIG. 1C. The mold was placed in a refrigerator and cooled to 4° C. for 20 min., and then the casts were fixed in 4% PFA overnight. The fixed protein blocks were removed from the mold and washed in 0.1 M PBS, FIG. 1D. After this fixing, the plasticized blocks were treated in exactly the same manner as any authentic fixed tissue, being dehydrated in increasing concentration of ethanol and then impregnated with wax, sliced and mounted on a slide, FIG. 1E.

Salmon Sperm DNA:

Approximately 100 mg of salmon sperm DNA was added to 0.5 mL of 15% gelatin and was mixed and incubated at 45° C. during the course of the day to generate a saturated solution. After centrifugation, to remove un-dissolved DNA, an aliquot of the solution was removed and the concentration of DNA was calculated using extinction coefficient ε₂₆₀=10,520 M⁻¹ cm⁻¹; (A+T) is 41.15% and (G+C) is 58.85% in salmon sperm DNA. The maximum concentration of DNA/gelatin obtained was 8.5 mM. DNA was visualized using either DAPI Slowfade Gold® (Molecular Probes/Invitrogen, Eugene, Oreg., USA) or YO-PRO-1® 1 μM dissolved in Fluoromount-G® (Southern Biotech, Birmingham, Ala., USA).

Conjugation of DNA Oligonucleotides:

Two oligonucleotide standards were obtained that were designed to form either a blunt-ended, or 3′-T-OH overhanging hairpin (Integrated DNA Technologies, Coralville, Iowa. Each had a thymine with a C6 hexane linker with a terminal amine, at the inflexion point. Two oligonucleotide probes, designed to ligate to blunt-ended and 3′-T-OH overhanging ended DNA breaks, were obtained which have a fluorophore present at the hairpin apex (Oligo Factory). The design of the standards and the corresponding oligonucleotide probe is shown in FIG. 2A and FIG. 2B. The blunt-ended standard contained the sequence GGTCTGGATCCAGCGC-3′ (SEQ ID NO:1); complement shown in (SEQ ID NO:2), while the blunt-ended probe contained the sequence 5′-GCTGAATTCAGACC (SEQ ID NO:3); complement shown in (SEQ ID NO:4). The T-overhanging standard included the sequence GGTCTGATCCGCT-3′ (SEQ ID NO:5); complement shown in (SEQ ID NO:6), while the T-overhanging probe included the sequence 5′-GCGCTGAATTCAGACC (SEQ ID NO:7); complement shown in (SEQ ID NO:8).

Suberic acid bis-succinimide was used to conjugate the oligonucleotide standards to gelatin. The blunt ended oligonucleotide standard, FIG. 2A, 109 nMoles, was diluted in 90 μL of water. To this was added 10 μL of freshly prepared 100 mM suberic acid bis-succinimide in ethanol. After 20 min, 600 μL of 3% gelatin in 10 mM phosphate, pH 7.0, was added, mixed and incubated at room temperature overnight. The gelatin was washed three times in 15 mL of 95% ethanol and then once in ice-cold water. The gelatin was rehydrated using 600 μL warm water, an aliquot was taken, and its UV spectrum recorded. As gelatin has very little absorbance in the UV region, the spectrum of the DNA is easily measured. A probe concentration of 112 μM, was recorded using the manufacture's extinction coefficient of 310,900 M⁻¹ cm⁻¹, indicating a conjugation efficiency was >60%. Nucleotide standards were prepared by diluting with 15% gelatin in the 0 to 20 μM range.

Conjugation of Proteins:

Fluorescently-labeled antibodies as well as avidin/streptavidin are widely used and are available from many commercial vendors. Moreover, many laboratories manufacture their own IgG-labeled probes using kits. It is important to be able to calibrate the fluorescent signals obtained using fluorescence microscopy with the actual level of specific binding protein. To this end, the methodology to prepare phantoms with known levels of a protein of interest, including IgGs and Avidin/Streptavidin is below. However, although these particular proteins were chosen for illustration purposes only, it should be apparent to one of ordinary skill in the art having benefit of the present teachings that any soluble peptide, protein, polypeptide, enzyme, and/or antibody can be entrapped in gelatin, and then covalently bonded to it via PFA fixation. Protein phantom blocks can be manufactured using a single, or mixture of proteins, in the same way that commercially-available proteins (some of which are often conveniently pre-labeled), are sold for use as standard protein markers (such as the ColorBurst™ Markers available from Sigma) in methodologies such as Western hybridization analyses, and the like.

Facile Preparation and Conjugation by PFA:

Using PFA, it was possible to conjugate soluble proteins directly to gelatin, as occurs during normal histological fixing, with little or no loss of protein during the fixing process.

A protein such as cytochrome c or Cy3-Streptavidin was mixed with molten gelatin to a final concentration of 15% gelatin at 45° C., and serially diluted, again in 15% gelatin at 45° C. 300-μL aliquots were dispensed into the wells of 96-well palates and the spectrum was taken. The plate was warmed and 200-μL aliquots were dispensed into the Pelco molds, and placed in a refrigerator for 1 hr. The casts were then fixed overnight in 4% PFA. The 200-μL phantoms had a thickness of 3 mm±200 μm and the concentration of the label was measured using absorbance spectroscopy.

Quantitative Preparation, Using Internal Standard to Measure Path-Length:

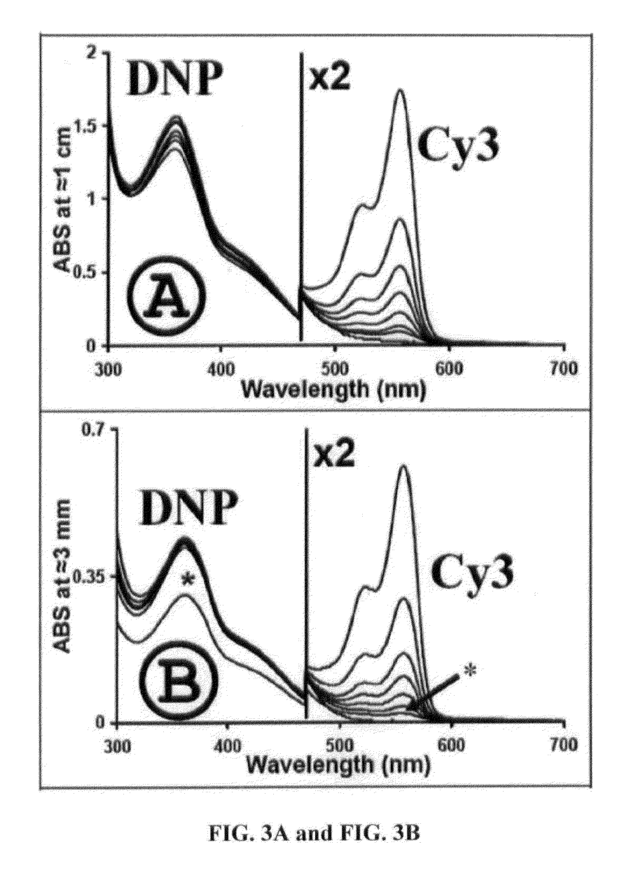

To know the levels of a chromophore in a solution, gel or solid, one needs to know the absorbance, the extinction coefficient and the path length. When blocks are cast that contain a chromophore/fluorophore attached to the gelatin or to a dissolved probe protein one can calculate the levels of this chromophore/fluorophore with using spectroscopy, from the absorbance, only if the path length of the cast block is known. It is clear that the thickness of a cast block is both variable and non-uniform. However, by pre-labeling gelatin with its own chromophore, one can determine the path length of the cast block, and can thus produce blocks where one knows exactly the levels of the entrapped probe. By labeling gelatin with dinitrophenyl hydrazine, it permitted the measurement of the path length of a PFA-fixed block. This was demonstrated in FIG. 3A and FIG. 3B, where the preparation shown, Cy3-Streptavidin phantoms, were used to construct a standard curve.

The stock concentration of a commercially obtained Cy3-Streptavidin conjugate (ZyMed, San Francisco, Calif. USA) was determined spectrophotometrically as 81 μM Streptavidin and 44 μM Cy3 (Streptavidin tetramer; ε₂₈₀=165,304 M⁻¹ cm⁻¹ and Cy3; ε₅₅₉=150,000 M⁻¹ cm⁻¹). 300 μL of the Cy3-Streptavidin was dissolved in 300 μL of 30% gelatin, mixed by vortex and centrifuged to remove bubbles. This was then mixed 1:1 with a solution of DNP conjugated gelatin, at 168 μM DNP, to a gelatin concentration of 15%. This generated a stock Cy3-Streptavidin solution that contained 15% gelatin, 84 μM DNP, 20 μM Streptavidin and 11 μM Cy3. This stock underwent a serial dilution from 5.5 μM to 150 nM Cy3 in 84 μM DNP-gelatin, and the optical spectra of 300-4, aliquots were recorded. The absorbance spectra are shown in FIG. 3A. 200-μL aliquots were then taken from each well and placed in molds, which were fixed and washed. The absorbance of the ≈3 mm phantom blocks was taken, FIG. 3B. The two series of absorbance spectra demonstrated the variability of path-lengths, as a result of the difficulty in dispensing the viscous gelatin solution accurately. In the wells, the DNP absorbance varied by ±6.8% and in the case of the phantoms by ±8%; but notice the one outliner (*) in FIG. 3B, which, without the use of a DNP internal reference, would have been incorrectly measured. By using the known concentration and extinction coefficient of DNP in each block, the exact path length of the individual blocks was measured, and from this, the concentration of the Cy3-Streptavidin was accurately calculated.

Conjugation Before Fixation:

Cytochrome c is a highly soluble redox heme protein with well-known spectral characteristics. Moreover, it often plays a critical role in apoptosis (Lartigue et al., 2008; Sharonov et al., 2005). Cytochrome c was used as a model protein to conjugate to gelatin, before PFA treatment. To definitively conjugate the cytochrome c before fixation the doubled-ended amine reactive six carbon linked reagent, suberic acid bis(N-hydroxysuccinimide ester), was used. By covalently bonding the cytochrome c to gelatin, prior to fixing, the inventors could ensure that the fixing process was not perturbing the properties of cytochrome c. 500 μL of 1 mM cytochrome c in water was mixed with 100 μL of 10 mM suberic acid bis-succinimide for approximately one minute and then with 3 mL of warm 3% gelatin and vortexed. The solution was allowed to incubate for 1 hr at 45° C. The proteins were precipitated using 14 mL of ethanol and the pellet was then twice washed with ice-cold water, to remove unbound cytochrome c and then rehydrated using 900 μL of water.

Rehydration at 45° C. took a number of hrs, with regular mixing. Large aggregates were removed by centrifugation and the cytochrome c was present homogenously throughout the gel, both before and after fixing, as demonstrated by the optical spectrum shown in FIG. 1D. The spectrum of oxidized and reduced conjugated cytochrome c was used to calculate the concentration, and to show that no optical perturbations had occurred. The overall coupling efficiency was 20%, and the reduced, oxidized and reduced minus oxidized cytochrome c spectrum showed that the conjugated cytochrome c was identical to that of the native spectrum.

A final concentration of 10% gelatin was used, rather than 15%, when using suberic acid-NHS to conjugate cytochrome c to gelatin to aid the cutting process of the waxed phantoms. It was found that the waxed phantoms blocks were difficult to cleanly slice when suberic-NHS acid treated gelatin at 15% was used.

Sample Treatment and Imaging

All tissue phantoms were sent to the pathology unit and dehydrated, waxed, and sectioned by the same staff using the same methods employed for standard tissue samples. “Techniques for Visualizing Gene Products, Cells, Tissues, and Organ Systems,” Chapter 16, in Manipulating the Mouse Embryo, 3rd edition, by Andras Nagy, Marina Gertsenstein, Kristina Vintersten, and Richard Behringer. Cold Spring Harbor Laboratory Press, Cold Spring Harbor, N.Y., USA, 2003.

Slides were de-waxed by incubation in xylene (2×) and then in 100%, 95%, 90%, 50% ethanol, all for 30 min. The slides were then washed 3× in 0.1 M PBS for 30 min. The slides were prepared for microscopy by covering the sample with mounting solution, a cover slip and sealed the edges using clear nail varnish. DNA was measured using DAPI (Slowfade Gold® with DAPI, Molecular Probe/Invitrogen) or 1 μM YO-PRO-10 in Fluoromount-G® (SouthernBiotech). Sigma Anti-DNP antibody produced in rabbit was used as primary and imaged using Alexa Fluor® 594 goat anti-rabbit IgG. (Invitrogen, Carlsbad, Calif.) Abcam mouse anti-cytochrome c IgG (Abeam, Cambridge, UK) was used a primary and imaged with Alexa Fluor® 488 goat anti-mouse IgG (Invitrogen, Carlsbad, Calif.) or as a FITC conjugate made in house. In all cases, the samples were blocked with 10% horse serum.

U87 Cells

U87, human glioblastoma, cells were obtained from the American TypeCulture Collection (ATCC) Manassas, Va. and grown as recommended in DMEM with penicillin/streptomycin and 10% FBS. Following 3 weeks of growth and splitting cells were plated into slide chambers (Lab-Tek Nalge Nunc International, Rochester, N.Y., USA) at 2×10⁵ cells per mL, and were allowed to grow thereon for 24 hrs until confluence was reached.

The following day, medium was removed, and 2 mL of fresh medium was added which contained either irinotecan at 250 μM dissolved in ethanol or an ethanol vehicle as control (i.e., 10 μL in 240 μL of medium). Twenty-four hrs after treatment, all cells were fixed with 4% PFA for one hr and washed 3× in 0.1 M PBS, incubated in 0.1% Triton-X100® for 6 min and then washed in 0.1 M PBS buffer. Each slide was assayed for double-strand DNA blunt-ended and overhanging 3′-OH ends, and counterstained for DNA with YO-PRO-1®.

In Situ Ligation: Blunt Ends and Overhangs

The inventors have developed a methodology for apoptosis detection in tissue sections, namely the in situ ligation assay (Didenko et al., 2004; Didenko et al., 2003; Didenko et al., 2002; Didenko, Ngo and Baskin, 2002; Didenko et al., 1999). The assay selectively labels a single type of apoptotic DNA damage, and can be used to selectively label double strand breaks with 3′-OH ends. It utilizes T4 DNA ligase, which attaches the hairpin shaped labeled oligonucleotide to cellular DNA with full double strand breaks, thus eliminating the possibility of labeling nicked or single stranded DNA. The in situ ligation assay is more specific for apoptosis than the conventional TUNEL technique, but less popular, partly because this assay does not permit the accumulation of properly quantified data.

The 3′-OH T specific overhang and blunt-ended DNA probes, shown in FIG. 2A and FIG. 2B, were ligated to the known oligonucleotide standard phantoms the amine apex oligoneuleotides, or alternatively, to fixed permeabilized U87 cells. The sections were pre-incubated in the ligation buffer without the probe (66 mM Tris-HCl, pH 7.5, 5 mM MgCl₂, 0.1 mM dithioerythritol, 1 mM ATP, and 15% polyethylene glycol-8000) to ensure even saturation. The buffer was aspirated, and the full ligation mix containing the ligation buffer with probe, 35 μg/μL, and 0.5 U/μL T4 DNA ligase (New England BioLabs; Ipswich, Mass., USA) was applied to the sections, which were then incubated in a humidified box overnight.

Controls consisted of probe-ligated phantoms, which were then incubated with either EcoRI or BamHI, in the appropriate buffer for 4 hrs, washed, cover-slipped using Fluoromount-G® (Southern Biotech), and sealed with nail varnish.

Epifluorescence Microscopy

The fluorescence signal was acquired using a Eclipse® TE2000-E fluorescent microscope (Nikon™, Shinjuku, Tokyo, Japan) equipped with a CoolSnap ES® digital camera system (RoperScientific, Trenton, N.J., USA) containing a CCD-1300-Y/HS1392×1040 imaging array cooled by a Peltier device.

Images were recorded using NIS-Elements software v3.13 (Nikon) and images were stored as both .tiff and .jpg files. Pixels of the .tiff file data, which have >10 times the pixel resolution of .jpg files, were analyzed using ImageJ public-domain software (Wayne Rasband, National Institutes of Health, Bethesda, Md., USA) (see Collins, 2007) and figures utilizing .jpg color images were analyzed using Photoshop® software (Adobe Corp., San Jose, Calif., USA).

Microscopic Calculations

The pixel dimensions of the inventors' microscope/camera have been calibrated by a representative of the manufacturer. At 100× magnification each pixel element represented an interrogated area of (0.061)² μm². At 40× magnification the interrogated area per pixel is (0.162)² μm². Given there are 1000 L in 1 m³, for each 1 μm of sample depth, the pixel volume at 100× magnification is 3.7×10⁻¹⁸ L (1×10⁻⁶×6.1×10⁻⁸×6.1×10⁻⁸ m³) and at 40× magnification was 2.62×10⁻¹⁷ L (1×10⁻⁶×1.62×10⁻⁷×1.62×10⁻⁷ m³).

At a solution concentration of 1 M, there are 2,167,920 molecules present in a 1 μm slice at 100× magnification. As 6-μm slices were used for phantoms, in a 6-μm phantom slice, there are ≈13.4 molecules per μM at 100× magnification, whereas at 40× magnification each pixel interrogates ≈95 molecules per μM.

Spectroscopy

Absorbance and fluorescence optical spectroscopy was performed using a Synergy® HT reader (BioTek U.S., Winooski Vt., USA). The fluorescence levels of FITC/Cy3-labeled proteins were calibrated in this instrument against known FITC/Cy3-gelatin conjugates. The well volumes in 96-well plates were corrected for a 1-cm path length. Instrument calibration was performed by comparing well volume against a cytochrome c solution in a cuvette with a 1-cm path length. All gelatin-probe absorbance measurements were corrected for the absorbance of the same concentration of unlabeled gelatin. The absorbance of the PFA-treated phantom blocks was measured using the same instrument.

Results and Discussion

FIG. 3A shows the optical spectra of DNP-labeled gelatin (84 μM) and various concentrations of Cy3-Streptavidin, measured in the well of a 96-well plate, path length≈1 cm. 200-μL aliquots were removed from each well and placed in molds containing the sample, which were fixed overnight in 4% PFA and then washed. The absorbance of the ≈3 mm phantom blocks was determined, FIG. 3B. The two series of absorbance spectra demonstrated the variability of path-lengths in the two types of measurement. In the wells, the DNP absorbance varied by ±6.8% and in the casts by ±8%; but one outlier (*) was observed in the data shown in FIG. 3B, which would have been incorrectly measured. Using the internal DNP standard, the absolute path length of the wells and of the phantom blocks was established and therefore the 560-nm Cy3 spectral signals were absolutely quantified.

Phantom Images

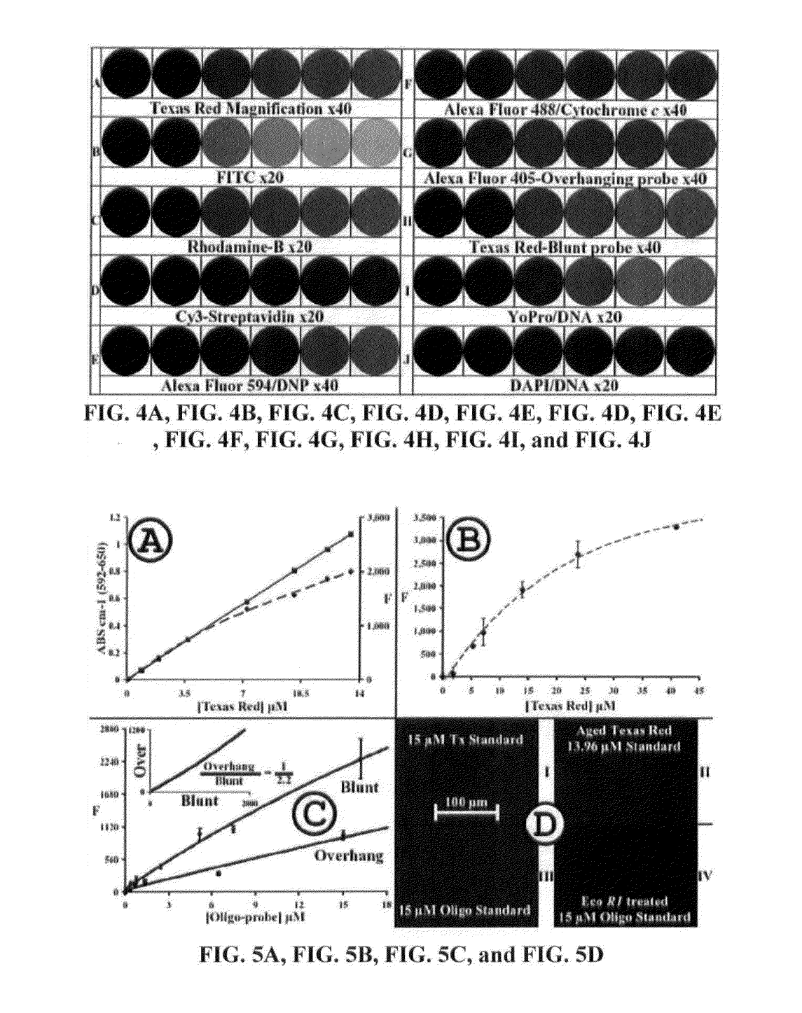

FIG. 4A, FIG. 4B, FIG. 4C, FIG. 4D, FIG. 4E, FIG. 4F, FIG. 4G, FIG. 4H, FIG. 4I, and FIG. 4J show a set of representative images of 20- to 50-μm thick discs taken from the .jpg images of various constructed phantoms. The first three phantoms, shown in FIG. 4A, FIG. 4B, and FIG. 4C, consist of dyes directly conjugated to gelatin, which have then undergone fixation, waxing, slicing, mounting, de-waxing, and rehydration. FIG. 4D illustrates the microscope images of Cy3-Streptavidin phantoms described in the previous section.

DNP is widely used as label, and in FIG. 4E labeling of DNP-gelatin phantom is shown with a primary goat polyclonal anti-DNP antibody that was visualized using an Alexa Fluor® 594-labeled mouse anti-goat monoclonal antibody. FIG. 4F shows how the cytochrome c phantoms were used to prepare a standard curve using an anti-cytochrome c specific antibody, which was in turn probed by a fluorophore attached to a secondary antibody. This pair of figures demonstrates that there is enough ‘room’ within the conjugated phantom matrix for a pair of independent antibodies to diffuse in and adhere to their respective epitopes.

FIG. 2A and FIG. 2B show how the DNA blunt and overhanging DNA standard ends were constructed, as well as the probes specific for each of those ends. FIG. 4G and FIG. 4H illustrate how the T4 DNA ligase in situ ligation reaction was used to attach the oligonucleotide probes to the oligonucleotide standards, in a concentration dependent manner. FIG. 4I and FIG. 4J show how salmon sperm DNA was used to construct DNA standard curves that can be probed with either YO-PRO-1® or the more traditional DAPI.

Standard Curves of Fluorophores and Oligonucleotides

FIG. 5A, FIG. 5B, FIG. 5C, and FIG. 5D present a complete data set to show how biologically relevant probes may be quantified, using Texas Red as an example. These figures show the relationship between the absorbance and fluorescence of Texas Red, conjugated to gelatin, measured in a plate reader. A series of Texas Red concentration standards, to an n=4, were prepared in 15% gelatin, and 200-4, aliquots were dispensed into the wells of a 96-well plate (see FIG. 1A, FIG. 1B, FIG. 1C, FIG. 1D, and FIG. 1E). The fluorescence (590/20 nm excitation and 645/40 nm emission) and absorbance (85,000 M⁻¹ cm⁻¹ at 597-650 nm) were recorded, and FIG. 5A shows the relationship between fluorescence and absorbance. There was some non-linearity, and the fluorescence signal was fitted using a hyperbolic function. The fact that this signal is hyperbolic, and not linear, is the type of information that can only be determined by the generation of calibration curves using methodologies outlined in this application.

FIG. 5B shows the relationship between fluorescence of 6-μm thick Texas Red-conjugated gelatin tissue phantoms and concentration. These slices were measured under the same conditions (40× magnification and 1.5-sec illumination time), that was used for the measurement of Texas Red labeled oligonucleotide bonded to biological samples during in situ ligation experiments. The curve was again hyperbolic, but it was clear that at concentrations below ≈15 μM, the relationship was almost linear. This hyperbolic behavior may be due to the fluorophore not truly being in solution, and by being conjoined to a rigid protein, had less movement in three-dimensional space in addition to the self-quenching that occurred with increasing fluorophore concentration, as previously discussed (Margoliash and Frohwirt, 1959). This non-linearity of signal vs. concentration is very important when interpreting signals from the measurement of probe levels in biological systems.

FIG. 5C shows the relationship between blunt and overhanging oligonucleotides, ligated to gelatin conjugated oligonucleotide standards. The design and pairing of the oligonucleotide standards and probes was shown in FIG. 2A and FIG. 2B. The Texas Red-labeled blunt-ended probes gave the same fluorescence/concentration dependence as the authentic Texas Red-conjugated gelatin phantoms measured under the same conditions. This permitted determination of the ligation efficiency of the selected probe, which was very high, ≈100%. FIG. 5C also shows the relationship for the Alexa Fluor® 405-labeled overhanging probe, ligated to the appropriate conjugated oligonucleotide standard. Here, the illumination time was increased from 1.5 to 5 sec, and yet the measurable signal was still less than that of Texas Red at any tested concentration.

The data in FIG. 5D demonstrated two important points: First, the Texas Red-conjugated waxed phantoms had a long shelf life. The series of Texas Red sliced phantoms used to generate the data set shown in FIG. 5B were stored in a slide case for more than 90 days. The signal was then compared against a pair of newly generated phantoms; a blunt-ended standard that was ligated with the selected Texas Red-labeled oligonucleotide probe, and an authentic Texas Red-conjugated gelatin phantom. There was virtually no detectable loss in Texas Red signal as a result of this aging process. Secondly, a blunt-ended, ligated, standard phantom was treated with the restriction endonuclease, EcoRI. The blunt/overhanging-ended standards and the probes were designed with restriction endonuclease cutting sites that permitted measurement of the nonspecific binding of the selected probes to the phantoms. Following treatment with EcoRI, and washing, the Texas Red levels were lowered to just above background (from 15 μM to ≦0.25 μM). This indicated that the Texas Red-labeled probe was ligated to the blunt-ended standard, which, in turn, was covalently linked to the gelatin. Measurement of the levels of the Texas Red probe, in the oligoprobe/oligostandard pairing indicated that the ligation efficiency was close to 100%. To reiterate; the levels of oligonucleotide standard in the phantom were measured via uv spectroscopy and known and the level of Texas Red probe attaching to this standard was measured by comparison to a Texas Red standard gelatin phantom, and the use of EcoRI was a negative control, showing that the binding of the Texas Red probe was not adventurate, and the oligoprobe was indeed ligated to standard.

Cytochrome c Standard Curves

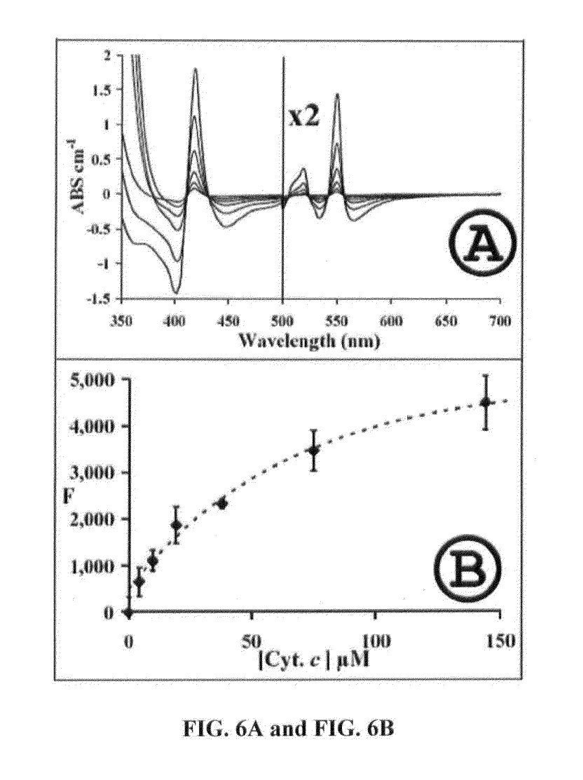

FIG. 6A and FIG. 6B show the dithionite reduced-minus-oxidized spectrum of suberic acid-NHS conjugated gelatin-cytochrome c, which demonstrated that cytochrome c could be visualized using a combined primary/labeled-secondary antibody system. The oxidized/reduced spectrum of the conjugated cytochrome c was indistinguishable from that of the native protein after baseline subtraction (Xu and Villalona-Calero, 2002). The reduced-minus-oxidized spectrum (FIG. 6A) had the classical peaks at 419 and 550 nm, and also the typical isosbestic points at 411, 432, 542 and 558 nm (Xu and Villalona-Calero, 2002).

There was some initial concern that high protein levels (when conjugated to gelatin) might restrict the movement of various probes into the protein matrix. To test such steric hindrances, cytochrome c was probed using a paired antibody combination (see FIG. 6B). Both the first anti-cytochrome c IgG and the second, fluorescently-labeled, anti-mouse IgG were able to diffuse into the tissue phantom and bind to their respective epitopes.

Application to Cell Imaging

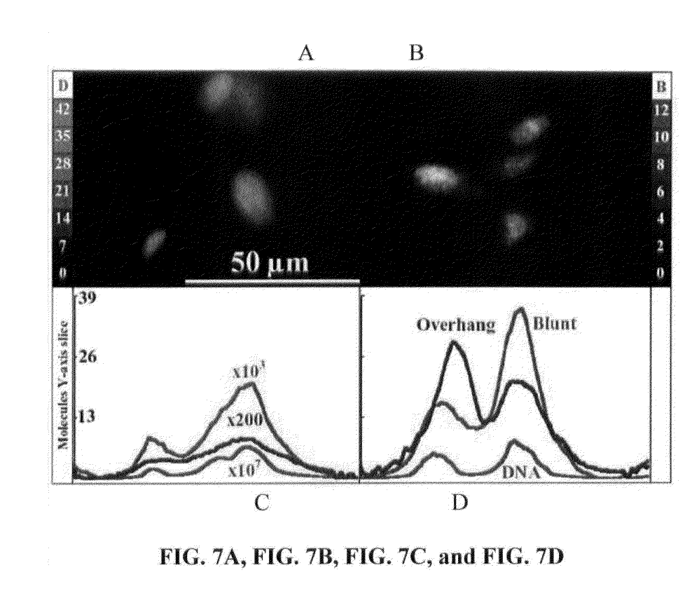

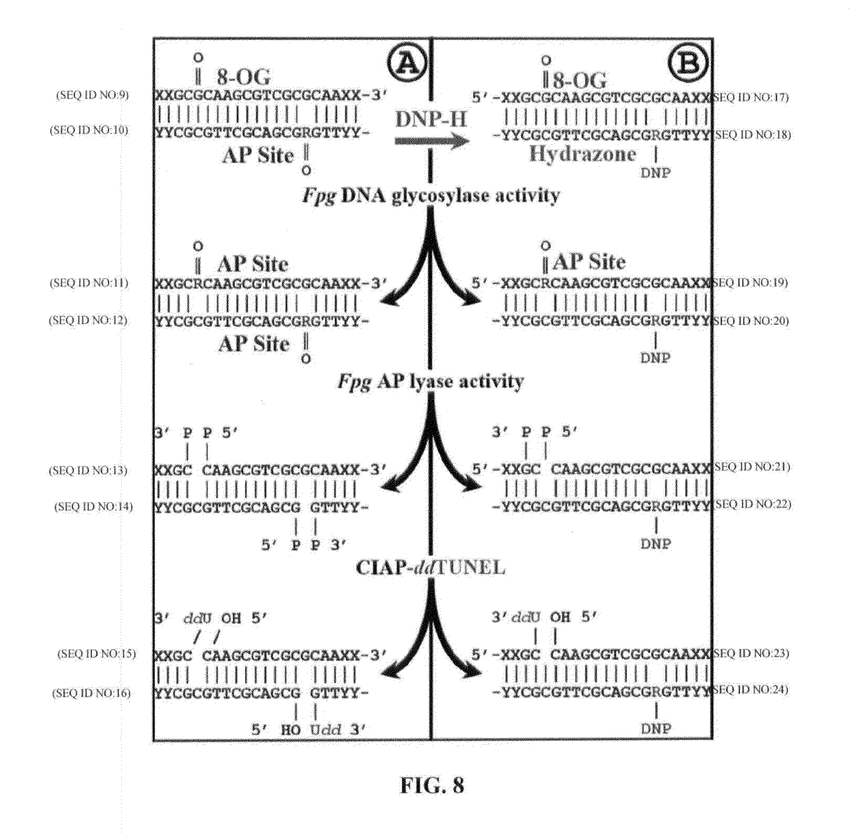

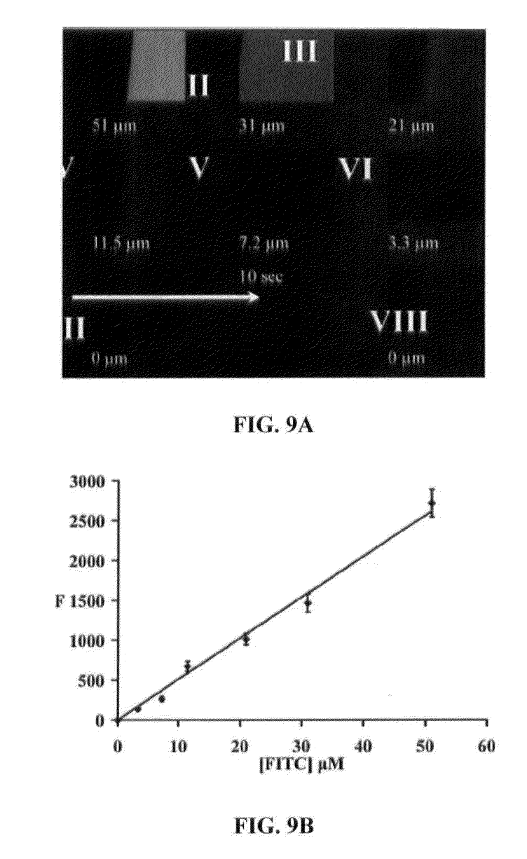

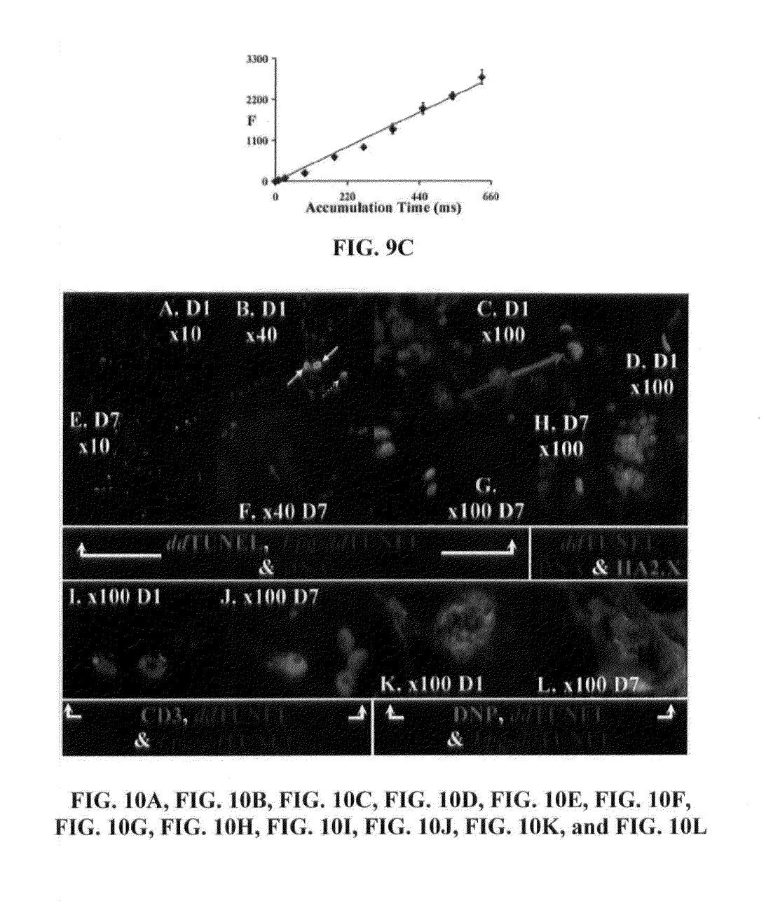

The ability to be able to measure the levels of specific types of DNA damage in cells is of enormous interest, especially in the field of cancer treatment. The majority of chemotherapeutic agents is targeted directly to DNA or to DNA repair enzymes, and so the ability to quantify DNA damage is useful not only in drug design, but are in the area of personalized medicine. To illustrate this point, the effects of the glioblastoma chemotherapeutic, irinotecan (a topoisomerase I inhibitor) were examined with respect to the number and type of DNA breaks in U87 cells. Analysis was performed using high-resolution .tiff image files, while the images presented for visualization were from lower-resolution .jpg files. The colors shown in FIG. 7 have undergone both background subtraction and intensity magnification so that the highest pixel represents an intensity of 255 color units.

In Situ Ligation: Blunt and Overhanging Probes

Control and irinotecan-treated cells were treated with the blunt-ended and 3′-T-OH-specific overhanging probes shown previously in FIG. 2A and FIG. 2B. The images in FIG. 7 showed YO-PRO-1®-labeled DNA in green, blunt-ended probe-labeled DNA in red, and overhang probe-labeled DNA in blue. The highest level of Texas Red that was measured corresponded to a phantom level of ≈10 μM, while the highest level of the overhanging Alexa Fluor® 405 probe that was measured corresponded to a phantom level of ≈3.2 μM. In the control cells, the average number of blunt-ended breaks was 190 per million base pairs and this increased to 285 per million base pairs after treatment with irinotecan. The effect on overhanging breaks was much more dramatic. The number of 3′-T-OH overhang ends increased from 43 per million to 119 per million basepairs following treatment. This change in the level of DNA breaks was consistent with what is known about the mechanism of irinotecan, the active metabolite of which (SN-38) is known to bind to the topoisomerase I/DNA complex, where it prevents the re-ligation of single-strand breaks in the DNA molecule caused by the enzymatic action of topoisomerase (Xu and Villalona-Calero, 2002).

A number of ways in which a fluorescent signal generated from a histological slide can be calibrated against an absolute standard have been demonstrated herein. The basis of the method is the property of hydrated gelatin to undergo a phase transition, within a temperature range suited to biological samples. Gelatin has little absorbance in the UV, visible, and near-IR spectral ranges, so it is especially suited for measuring the presence of chromophores in these regions of the spectrum. The simplest methodology for creating fluorescent tissue phantoms is to conjugate the dye directly to the gelatin. Alternatively, it is also possible to link antigens, such as FITC or proteins, to the gelatin. The use of a compound such as FITC is doubly useful, since an FITC-gelatin standard can also be used to assay any labeled secondary antibody, simply by using a murine anti-FITC monoclonal antibody. Results have demonstrated that standard curves for paired antibodies worked quite well in the gelatin phantom systems disclosed herein (see e.g., FIG. 3).

The ability to link and quantify oligonucleotides in gelatin means that it is now possible, for the first time, to quantify the levels of blunt-ended and T 3′-OH overhanging DNA breaks in cells.

Although biotin is the most widely used label for many different imaging techniques, its usefulness in creating phantoms according to the present methods appears limited. Biotin-phantoms were prepared, and then tested using the Surelink™ biotinylation method (KPL, Gaithersburg, Md., USA). However, it was found that the biotin was destroyed during the histological fixing and waxing process. While the biotin appeared to survive the initial PFA treatment, it was subsequently destroyed. Biotinylated phantoms were also prepared using a different methodology, in which mouse anti-FITC IgG was biotinylated, added to FITC-gelatin phantoms, and then labeled the biotin using Cy3-Streptavidin. This method, however, was more complicated and more costly than directly conjugating the Cy3-streptavidine to the gelatin, thereby limiting the usefulness of biotin in the methodology.

### Example 2

### Direct and Quantitative Measurement of Blunt and Overhanging DNA Ends

The present example describes a method of quantifying the levels of blunt- and overhanging-DNA ends using a method that employs oligonucleotide standards bound to gelatin slices. Using these tissue ‘phantom’ standards, a ligating efficiency of essentially 100% and a background staining level of <5% of the typical signal has now been achieved. The methodology's ability to label apoptotic nuclei and apoptotic inclusion bodies has been successfully validated using rat mammary gland, from Days 1 and 7 of involution. Moreover, the various types of DNA damage that occur in human glioblastoma U87 cells following exposure either to reactive oxygen stressing agents (such as H₂O₂ and Paraquat), or to one of the three chemotherapeutic agents routinely used for treating this disease (carmustine [BCNU], temozolomide, or irinotecan) have also been characterized.

The present ligase-based assay for selective detection of specific markers of necrosis/apoptosis in tissue sections utilizing oligonucleotide probes employs biotinylated, oligonucleotide, hairpin probes to detect the products of internucleosomal enzymatic DNA cleavage. Both blunt- and overhanging-DNA 3′-OH ends were detectable, and the typical products of DNase I type activity using in situ labeling of double-stranded DNA breaks. This method has many advantages over conventional terminal transferase-based labeling assays that stain apoptotic, necrotic and transiently damaged cells. Previous methods employing blunt-ended or over-hanging probes and T4 DNA ligase have been shown to offer much higher discrimination in labeling only cells which are undergoing DNA damaging DNase I activity. However, these biotinylated probes lacked signal quantification because the visualization was performed using avidin/streptavidin, each tetramer of which can bind between 1 and 4 biotin units. Moreover, the ligation efficiency of such earlier methods was unknown, as they were never tested against samples with known levels of either blunt-ended or overhanging standards.

In sharp contrast, the inventive methods described herein overcome the inherent limitations of conventional TUNEL-based methods. In the new probe design, the biotin reporter molecule has been eliminated, and a fluorophore (e.g., Texas Red or Alexa Fluor® 405) has been attached to the oligonucleotide probe instead. The oligonucleotide standards were conjugated to gelatin using suberic acid bis-succinimide. The new probes and standards were created with restriction endonuclease cutting sites incorporated into their sequence, so that background levels of fluorescence could also be quantified.

Materials and Methods

Preparation of Oligonucleotide Standards and Oligonucleotide Probes

Suberic acid bis-succinimide (Sigma-Aldrich) was used to conjugate the oligonucleotide standards to Type A pig skin gelatin (Sigma-Aldrich). The blunt-ended oligonucleotide standard was 109 nMoles (supplied as a lyophilized, buffered powder), and diluted in 90 μL of water. To this, 10 μL of freshly-prepared 100 mM suberic acid bis-succinimide (in ethanol) was added. After 20 min, 600 μL of 3% gelatin [in 10 mM phosphate (pH 7.0)] was also added, mixed, and then incubated at room temperature overnight. The gelatin was washed three times in 15 mL of 95% ethanol, and then once in ice-cold water. The gelatin was rehydrated using 600 μL warm water. A probe concentration of 112 μM was recorded using the manufacturer's reported extinction coefficient of 310,900 M⁻¹ cm⁻¹, which indicated a conjugation efficiency of >60% was achieved. Nucleotide standards were prepared by diluting with 15% gelatin in the 0 to 20 μM range. The gelatin-oligonucleotide standards were then melted at 45° C., cast into 20-cavity embedding silicone molds (PELCO®, Ted Pella, Inc, Redding, Calif., USA), cooled to 4° C., fixed in 4% paraformaldehyde, and subjected to the same histological procedures as the tissue samples. The overhanging standard was prepared in a similar manner.

Two oligonucleotide standards that were designed to form either a blunt or 3′-T-OH overhanging hairpin were commercially obtained (Integrated DNA Technologies, Inc., Coralville, Iowa, USA). Each had a thymine with a C₆ hexane linker with a terminal amine, at the inflexion point. Two oligonucleotide probes were designed to ligate to blunt-ended and 3′-T-OH overhanging-ended DNA breaks, which had a fluorophore present at the hairpin apex, via a hexane-amine linker (Oligo Factory, Holliston, Mass., USA). The conjugation of Texas Red was performed by Oligo Factory, while the conjugation of NHS-Alexa Fluor® 405 (Invitrogen) was performed in the inventors' laboratory using the methodology recommended by the manufacturer.

In Situ Ligation: Blunt-Ends and Overhangs

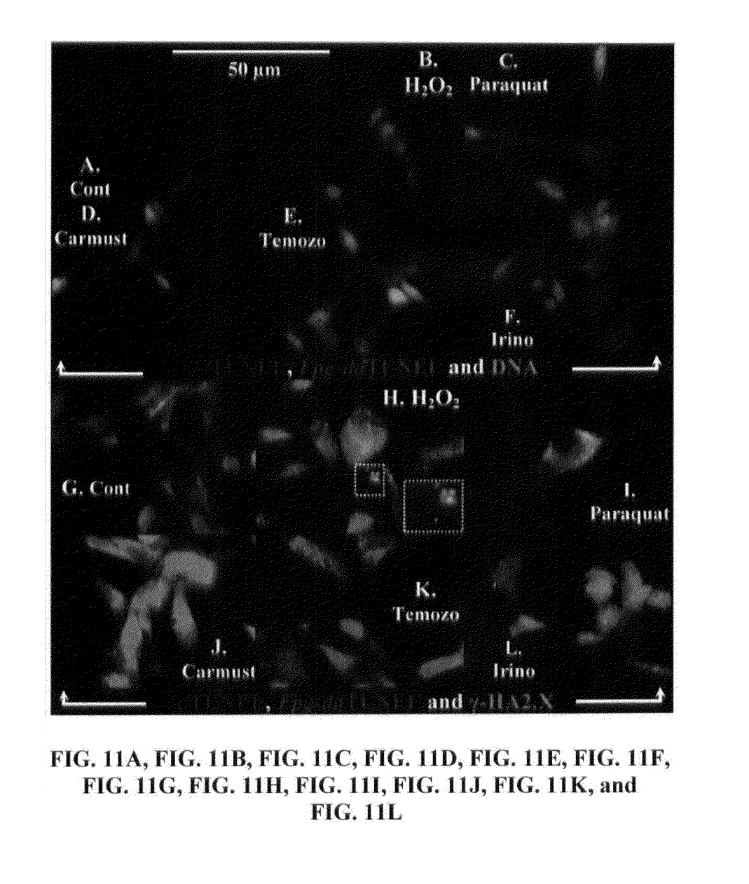

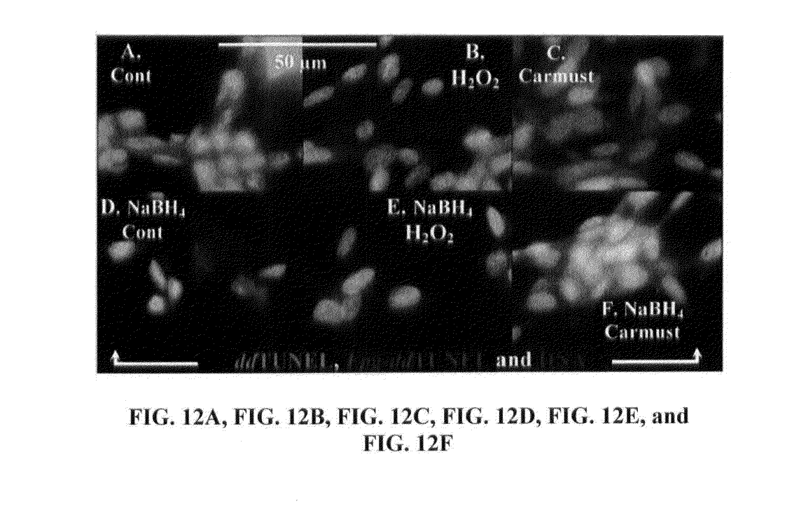

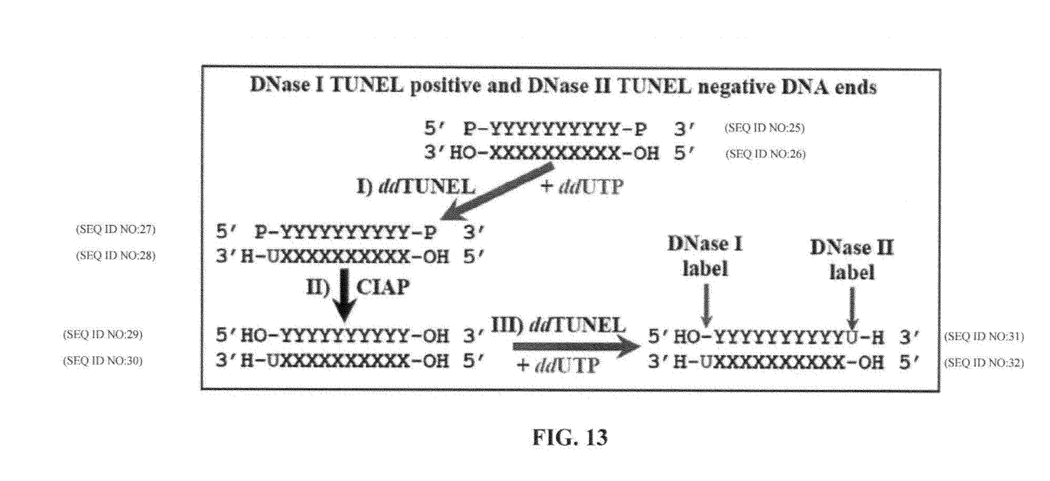

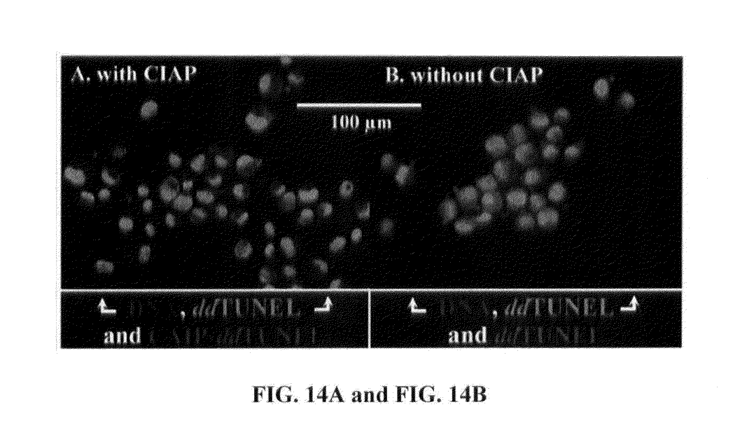

The 3′-OH T-specific overhanging- and blunt-ended DNA probes shown in FIG. 11B were ligated to oligonucleotide standard phantoms, mammary gland or to fixed, permeabilized U87 cells. The sections were pre-incubated with ligation buffer in the absence of the probe (66 mM-Tris HCl, pH 7.5, 5 mM MgCl₂, 0.1 mM dithioerythritol, 1 mM ATP, and 15% polyethylene glycol-8000) to ensure even saturation. The buffer was aspirated and the full ligation mix [ligation buffer+probe (35 μg/μL) and T4 DNA ligase (0.5 U/μL)] was applied to the sections, which were then incubated in a humidified box overnight. Controls consisted of probe-ligated phantoms, which were then incubated with either EcoRI or BamHI, in the appropriate buffer for 4 hrs, washed, cover-slipped using Fluoromount-G™ (SouthernBiotech), and then sealed with nail varnish.

Sample Treatment and Imaging

All tissue phantoms were dehydrated, waxed, and then sectioned using the same methods as used for conventional tissue sample preparation. All fixed paraffin embedded slides were de-waxed in xylene (2×) and then in 100%, 95%, 90%, and 50% ethanol, each for 30 min. The slides were then washed in 0.1 M PBS for 30 min, and prepared for microscopy by covering the sample with mounting solution and a cover slip, and then sealed using clear nail varnish. DNA was measured using 1 μM YO-PRO-1® (Invitrogen) dissolved in Fluoromount-G™ (SouthernBiotech).

FITC-labeled Anti-H2A.X histone monoclonal antibody (BioLegend, San Diego, Calif.) was used as an internal control for DNA breaks, at a 1/50 dilution after the samples were blocked with 10% horse serum.

Rat Mammary Gland

Slides containing three, 5-μm thick sections, fixed-paraffin embedded slices of rat mammary gland on Day 1 and Day 7 of involution were used as a model system of apoptosis (RP-414-N1 and RP-414-N7, Zyagen, Inc., San Diego, Calif., USA). All samples underwent treatment with Sudan Black to remove autofluorescence signals, as suggested by Romijn et al., (1999). A 0.3% Sudan Black (wt./vol.) in 70% Ethanol (wt./vol.) was applied to the slide for 10 min after ligation/antibody application, and was then washed 8× in 0.1 M PBS. No change was seen in the response of the FITC, Alexa Fluor® 405 or Texas Red using this methodology.

U87 Cells

Human glioblastoma cells (U87) (American Type Culture Collection, Manassas, Va., USA), and grown as recommended in DMEM with penicillin/streptomycin and 10% FBS. Following 3-weeks' growth and splitting, 5 mL of cells were plated into microscope slide chambers (Lab-Tek) at 2×10⁷ cells per mL, and were allowed to grow in the slide chambers for 24 hrs, until they became confluent. The following day the spent medium was removed and 2 mL of fresh medium (containing either a mixture of 1 mM H₂O₂, 300 μM Paraquat, 25 μM temozolomide, 2.5 μM Carmustine, and 250 μM irinotecan, or an ethanol vehicle control) was added. The particular concentration of each reagent was chosen as it represented the LD₅₀'s of these cells, measured over a 24-hr incubation time. 24 hrs after treatment, all cells were fixed with 2% PFA for 1 hr, washed 3× in 0.1 M PBS, then incubated in 0.1% Triton X-100® for 6 min and finally washed in 0.1 M PBS buffer. Each slide was assayed for double-strand blunt (red) and overhanging (blue) DNA damage, and counterstained for DNA with YO-PRO-1® (green) or for γH2A.X with an FITC-labeled IgG antibody (green).

Epifluorescence Microscopy

The signal was acquired using a Nikon Eclipse TE2000-E fluorescent microscope equipped with a CoolSNAP ES® digital camera system (Photometrics/Roper Scientific, Tucson, Ariz., USA) containing an CCD-1300-Y/HS1392×1040 imaging array cooled by a Peltier device. Images were recorded using Nikon NIS-Elements software, and images were stored as both .tiff and .jpg files. Pixels of the .tiff file data, which have >10× the pixel resolution of .jpg files, were analyzed using ImageJ public domain software (Wayne Rasband, NIH, USA) (see Collins, 2007); figures utilizing .jpg color images were analyzed using Adobe Photoshop™.

Results and Discussion

Tissue Phantom Standards

The ligation of the Alexa Fluor® 405-labeled 3′-T-OH overhanging probe to its gelatin phantom standard, and the relationship between epifluorescence and probe concentration was performed. Images were recorded for labeled phantoms with or without incubation with the restriction endonuclease, EcoRI. Treatment of gelatin-standard-probe with EcoRI reduced the fluorescence level by >95%, to that of the background. The resulting plots gave a standard curve that was obtained using a number of blunt and overhanging standards, following ligation with their respective probes. The use of these probes permitted calibration of the system, and facilitated calculation of the absolute levels of both probes in a variety of tested samples.

Apoptosis in Rat Mammary Gland

Cessation of milk removal leads to rapid changes in the mammary tissue and initiation of the process of mammary involution. Mammary involution in the rat is characterized by a rapid loss of tissue function and degeneration of the alveolar structure and massive loss of epithelial cells, due to apoptosis, that has been monitored by TUNEL (see, e.g., Colitti et al., 2009; Bagheri-Yarmand et al., 2009; and Lacher et al., 2003). Morphology consistent with apoptotic cell death was observed in the rat mammary gland the first day of involution. The nucleus and cytoplasm condense, the chromatin becomes fragmented and marginated, and apoptotic bodies were formed. Results illustrated the appearance of DNA breaks in rat mammary tissue during this natural process of organ sculpting. Autofluorescence was quenched in the samples from the rat tissue. In the unlabeled Day 1 sample, a composite of images were captured using blue, green and red filters, and showed much autofluorescence (caused by Lipofuscin and other naturally-occurring fluorophores), which made any analysis of the specific fluorescence levels impossible. These signals could be quenched, however, by staining the sections alter ligation/immunolabeling with Sudan Black B, which completely blocked this autofluorescence. The resulting micrographs revealed the imaging of DNA, blunt-ended DNA breaks and 3′T-OH overhanging breaks of rat mammary tissue (recorded at 10×, 40× and 100× magnification). A few apoptotic nuclei were observed on Day 1, but there was also the appearance of small apoptotic bodies that contained blunt, overhanging DNA breaks, and the remnants of nuclei. By Day 7, there had been extensive organ sculpturing with the ducts collapsing, the presence of many apoptotic nuclei and the presence of large apoptotic bodies. Particularly noteworthy is that there were three types of apoptotic nuclei present—some apoptotic nuclei were fragmented by DNases that created blunt-ends, some fragmented by DNAses that generated overhanging cuts, or a combination of both forms of fragmentation.

In mammalian cells, DNA double-strand breaks induce phosphorylation of serine-139 in the C-tail serine-glutamine-glutamate motif of the histone variant H2A.X, creating γ-H2A.X, which acts as a signal for DNA repair or for apoptosis (see, e.g., Solier and Pommier, 2009). The inventors tested whether the probes were measuring DNA breaks in this organ model of apoptosis, using FITC-labeled anti-γ-H2A.X with blunt/overhang ligated oligonucleotide probes. It was shown that in Day 1 tissue there was significant co-localization of the probes with γ-H2X, especially in the apoptotic bodies. γ-H2A.X, blunt and overhanging DNA breaks in tissue harvested on Day 7 were also observed. These results demonstrated that more histone was associated with the apoptotic bodies than with nearby cells that were undergoing apoptosis, that the apoptotic bodies contained DNA, γ-H2A.Y and both types of DNA breaks, and that the observed signals were not due to artifacts.

The difference in the levels of blunt-end breaks between control cells and those of temozolomide-treated cells was greater than 30×, and the difference in the overhang breaks in irinotecan-treated vs. control cells was greater than 50×. This range was greater than the dynamic range of the eye in judging background color in colored images, so .jpg files were deliberately overexposed in case of chemotherapy agents. There were very few DNA breaks in the control cells, but the background level of red blunt-end breaks was more apparent, as was the presence of γ-H2AX, which was mostly found in the cytosol. Hydrogen peroxide-treated cells showed the classical morphological changes associated with apoptosis, including the formation of blebs. Of note was the distribution of DNA throughout the cells; it appeared that the DNA in the small apoptotic blebs labeled with blunted ended probe, and with very little overhanging probe. Conversely, raised levels of γ-H2A.X in the cytosol correlated very strongly with overhanging DNA breaks, but not with blunted-ended DNA. Paraquat (which generates superoxide radicals in mitochondria that can then be converted into hydrogen peroxide) had a completely different death signature from that of H₂O₂. This was somewhat surprising, and may explain the previous antioxidant/ROS death studies in other cell systems (see e.g., Samai et al., 2007, and references contained therein). There was no blebbing apparent in any of these paraquat treated cells, whereas approximately 2% of the control cells showed evidence of apoptotic cell death and blebbing. Moreover, blunt-ended cuts were mostly restricted to the nucleus, whereas the overhang cuts were mostly found in the cytosol as was 65% of the γ-IGAX. Treating cells with carmustine or temozolomide induced massive increases in blunt-ended breaks, and increased the levels of overhangs by more than an order of magnitude.

There are important differences between the mechanisms of cell death that result from these two DNA alkylating agents. In carmustine-treated cells, blunt-ended cuts are mostly restricted to the nucleus and overhangs are found 2:1 in the cytosol, whereas with temozolomide, not only is the distribution of blunt-ended cuts more even between the two compartments, so too is the distribution of the levels of overhangs. The most striking difference was a 10-fold difference in cytosolic γ-H2A.X levels in carmustine-treated cells, when compared to those treated with temozolomide. These levels could not be correlated with the numbers of total breaks, or to the ratio of blunt/overhang. Irinotecan induced both overhang and blunt-ended breaks, but at a ratio of 5:1 in favor of the former. The blunt ends were localized in the nucleus and in apoptotic vesicles, but while these vesicles contained DNA, they showed relatively low levels of γ-H2AX.

Summary

This example demonstrates a methodology for the labeling of DNA-OH 3′ blunt ended and overhanging breaks, using T4 DNA ligase and oligonucleotide probes. These probes can be combined with other probes, such as YO-PRO-1® or FITC-IgG to reveal information about the mechanism of DNA hydrolysis during cell death. The use of rat mammary gland on Days 1 and 7, post-involution, validated the specificity of the probes and showed the fine detail of apoptosis that occurs in a natural form of organ sculpting. Moreover, it has been demonstrated that the apoptotic bodies contain DNA bearing both types of DNA cuts and the probes are co-localized with histone γ-H2AX.

The investigation of the effects of ROS and chemotherapy agents on U87 cells demonstrated that the processes that occur during cell death are insult specific; and that by using DNA standard phantoms quantification of the type and number of DNA breaks in each nucleus could be achieved. Moreover, it was shown that in these cells, without treatment, the average number of blunt end breaks was ≈145 per million base pairs and of overhang was ≈40 per million base pairs. Compare this to the non-apoptotic cells in the breast tissue where the levels are ≈60 per million base pairs and of overhang is ≈10 per million base pairs, respectively. This indicated that the immortalized cancer cells have poor housekeeping with normal cells. The effects of the stressors was somewhat surprising; all the agents increased the overall levels of DNA breaks, but most importantly changed the Over/Blunt ratio from a resting level of 0.3 to as high as 2.5, which suggested that the DNA repair pathways for overhanging breaks are more easily saturated than are the pathways for repairing bunt ended breaks.

It is interesting to note the difference in the behavior of cells treated with carmustine and temozolomide seen in this study. While the two drugs had similar effects on the generation of blunt ended and overhanging DNA breaks (with approximately 900 blunt-ended and 350 overhanging cuts per million base pairs present in both cases), carmustine, however, was associated with the transfer of γ-H2A.X into the cytosol, while little or no transfer was seen when using temozolomide. In addition, carmustine caused the formation of small blebs, containing very high concentrations of DNA with blunt-ended breaks and histone, whereas there was little evidence of blebbing following temozolomide treatment and the small features which may be vesicles appear to contain little histone, but do containing overhanging DNA. The observations are in no way definitive of the complexities of the death pathways that were investigated, but serve to illustrate one important method in which the new assay may be utilized.

### Example 3

### Quantification of DNase Type I and II Ends and Oxidized/Acylated Bases

In the present example, a quantitative assay has been developed by substituting labeled ddUTP in place of dUPT in the conventional TUNEL assay, and a protocol developed to permit, for the first time, a TUNEL-based assay to be used in a quantifiable manner. The inventors are also the first to demonstrate how such a ddTUNEL assay can be combined with phosphatase treatment to detect and specifically quantitate the levels of DNase Type II activity in a single sample.

The ddTUNEL assay described herein has been combined with the base-modification repair enzyme, formamidopyrimidine-DNA glycosylase (Fpg), to interrogate the levels of modified DNA in tissues or in fixed, cultured cells. Using rat mammary gland, from Days 1 and 7 of involution, the inventors have validated the new methodology's ability to label apoptotic nuclei and apoptotic inclusion bodies. In addition, the types of DNA damage and modification that occur in a human glioblastoma cell line (such as U87 cells), have been investigated following exposure to reactive oxygen stressing agents, H₂O₂ and Paraquat, alkylating agents, and the topoisomerase I inhibitor, irinotecan.

Materials and Methods

Below are exemplary reagents and protocols used in exemplary embodiments of one or more aspects of the present invention:

Deparaffinization/Background Quenching/Rehydration Reagents

Reagents: Xylene; Ethanol, anhydrous denatured, histological grade.

Wash buffer: 1×PBS/0.1% Triton X-1000 (1×PBST). To prepare 1 L add 100 mL 10×PBS to 900 mL H₂O, 1 mL Triton-X100® and mix thoroughly.

10×PBS (Thermo Fisher Scientific Inc, Rockford, Ill., USA)

Lipofuscin/background fluorescence quenching: 0.3% Sudan Black/70% ethanol.

ddTUNEL Reaction Buffer

ddTUNEL reaction buffer was prepared fresh daily by diluting a previously-frozen) stock solution of ddTUNEL buffer 1:5, and solution of cobalt chloride 1:25.

Stock reaction buffer, 5×: 125 mM Tris-HCl, 1 M sodium cacodylate, 1.25 mg/mL BSA, pH 6.6. Stock reaction CoCl₂ solution, 25×: 25 mM cobalt chloride.

It was found that ddUTP labeled with PromoFluor-594 was a very good substrate for the ddTUNEL assay. Using a typical epifluorescence microscope filter set up (DAPI, FITC and Texas Red) the following combinations were tested:

1) DAPI, biotin-ddUTP/Streptavidin-Alexa Fluor® 488 and PromoFluor-594 ddUTP; and

2) Biotin-ddUTP/Streptavidin-Alexa Fluor® 405, YO-PRO-1® (Invitrogen) and PromoFluor-594 ddUTP.

Invitrogen and PromoKine supply their Alexa Fluor® or PromoFluor® fluorophores, respectively, in amine-reactive (NHS)— forms, which permit the phantom dye standards of the present invention to be easily prepared.

Reduction of Oxo-DNA bases

Each sample was incubated in 25 mM sodium borohydride in 70% ethanol for 30 min. The sample was then washed twice in 0.1 M PBS. Typically, a slide box was employed, and it was half-filled with reducing solution so that half the sample(s) on the slide were reduced.

DNP Derivatization of Carbonyls and Elimination Schiff Bases

Each sample was incubated in 15 mM DNP-H in 2.5 M HCl for 30 min and was then washed twice in 0.1 M PBS.

Preparation of Tissue Sections

A. De-paraffinize/quench background fluorophores/rehydrate tissue sections.

B. Place tissue sections in two washes of xylene for 10 min each.

C. Place tissue sections in two washes of 100% ethanol for 10 min each.

D. Place tissue sections in two washes of 95% ethanol for 10 min each.

E. Place tissue sections in two washes of 70% ethanol for 10 min each.

F. Incubate tissue sections for 5 min with 0.3% Sudan Black/70% ethanol to remove background fluorescence (see, e.g., Romijn et al., 1999).

G. Place tissue sections in two washes of 50% ethanol for 10 min each.

H. Place tissue sections in two washes of 20% ethanol for 10 min each.

I. Place tissue sections in four washes of PBST for 10 min each.

Finally, using a hydrophobic liquid repellant barrier pen (PAP pen; Cat. No. 9804; Scientific Devices Laboratory, Des Plaines, Ill., USA), carefully draw around tissue sections to minimize area incubated with the subsequent reaction mixtures.

Preparation of Cells in Multi-Well Plates or in Microscope Slide Tanks

Grow cells (e.g., in microscope slide tanks or in one or more wells of a multi-well microtiter plate) in the presence of one or more effectors (e.g., the chemotherapeutic agent carmustine). Remove cell culture medium, add ice-cold buffered 1% PFA and leave overnight at 4° C. Wash cells in wells/tanks four times with PBST for 10 min each.

ddTUNEL Assay for the Detection of 3′OH Ends

1. Wash samples twice in ddTUNEL reaction buffer for 10 min each.

2. Incubate sections in ddTUNEL reaction solution for 1-2 h at 37-40° C. in humidified chamber or overnight at room temperature.

For 5-mm thick tissue sections, 20 to 60 mL ddTUNEL reaction mixture per section is desirable, while 25 mL ddTUNEL reaction mixture is employed per well in a multi-well (e.g., a 96-well microtiter) plate format.

3. Remove and if using all 96-wells, store used ddTUNEL reaction solution at 4° C.

4. Perform two 10-min washes in PBST.

5. Incubate with fluorescently labeled avidin or streptavidin (5-30 mg/mL) for 30 min.

6. Repeat two 10-min washes in PBST.

These samples are then used for CIAP-ddTUNEL.

CIAP-ddTUNEL Assay for the Detection of 3′PO₄ Ends

NEBuffer3 wash and reaction solutions; 50 mM Tris-HCl, 100 mM NaCl, 10 mM MgCl₂, 1 mM dithiothreitol, pH 7.9, and 100 U/mL of CIAP (New England BioLabs).

1. Perform two 10-min washes with 1× NEBuffer3 (New England BioLabs).

2. Incubate sections with 20 U/mL CIAP in 1× NEBuffer3 for 1-2 hr at 25° C.

3. Remove and wash twice with PBST for 10 min per wash.

4. The sample can be used in a second round of ddTUNEL. The previously stored/recycled ddTUNEL reaction solution can be used here.

5. Perform two 10-min washes with 1× NEBuffer3.

6. Incubate sections with 20 U/mL CIAP in 1× NEBuffer3 for 1-2 hr at 25° C.

7. Remove and wash twice with PBST for 10 min per wash.

8. The sample can be used in a second round of ddTUNEL. The previously stored/recycled ddTUNEL reaction solution can be used here.

9. After CIAP-ddTUNEL is complete, each 3′PO₄→3′OH can be visualized using fluorescently-labeled avidin/streptavidin. In a typical assay, half the samples are capped at the biotinylated 3′PO₄ 3′OH ends using 10 μg/mL unlabeled, native avidin for 30 min. These samples are then used for Fpg-ddTUNEL.

Each sample, having previously undergone ddTUNEL, was washed and incubated with NEBuffer3 for 30 min and then with ≈50 μL of the same buffer containing 100 U/mL of CIAP (Sigma-Aldrich) for ≧2 hrs and the newly-generated, 3′PO₄→3′O, ends were labeled by ddTUNEL.

CIAP-ddTUNEL-positive controls. As levels of 3′PO₄ were typically very low in all of the samples investigated, positive 3′PO₄ controls were prepared using authentic DNase II. Fixed, permeabilized, and washed U87 cells were treated with 10 Units/mL of DNase II (Sigma-Aldrich) for 30 min at 37° C. in 80 mM sodium acetate buffer containing 25 mM magnesium chloride (pH 4.6). The levels of authentic 3′OH ends were labeled in a first ddTUNEL round with FITC-avidin/Biotin-ddUTP, and then half of the samples were incubated in buffer containing CIAP, and the other half in buffer alone. They then underwent a second round of ddTUNEL with 3′PO₄→3′OH ends labeled with Texas Red-avidin/biotin-ddUTP.

Fpg Assay

The sample was washed twice in 10 mM HEPES, 10 mM NaCl, 2 mM EDTA and 0.1% BSA and then ≈50 μL of the same buffer containing 100 U/mL of Fpg was applied to each of the sections, which were then incubated in a humidified box for ≧4 hr. The sample was washed twice in 100 mM PBS, and then twice in NEBuffer3. Approximately 50 μL of the same buffer containing 100 U/mL of CIAP was then applied to each section, and incubated for ≧2 hrs.

Fpg-ddTUNEL Assay for the Detection of Modified Bases

Washing and reaction buffers: 10 mM Tris, 100 mM KCl, 2 mM EDTA, 0.1 mg/mL BSA, pH 7.5. (Mut M/Fpg Buffer; Affymetrix, Santa Clara, Calif., USA).

1. After completing both ddTUNEL and CIAP-ddTUNEL, each sample is washed twice in Fpg-buffer (Affymetrix) for 10 min.

2. Samples are then incubated in 100 U/mL Fpg (Affymetrix) in Fpg Buffer for 2 hr at 37° C. (or, alternatively, overnight at room temperature).

3. Following incubation, samples are washed twice in PBST (10 min each).

4. Samples are then incubated with fluorescently-labeled avidin/streptavidin (5 to 30 mg/mL) for 30 min.

5. Two final washes of the samples in PBST are performed for 10 min each.

Following ddTUNEL and CIAP-ddTUNEL, capping 3′OH/3′PO₄ ends, samples were washed twice in 10 mM HEPES, 10 mM NaCl, 2 mM EDTA and 0.1% BSA and then ≈50 μL of the same buffer containing 100 U/mL of Fpg was applied to each of the sections, and then incubated in a humidified box ≧4 hrs. Each sample was washed twice in 100 mM PBS, twice in NEBuffer3 and ≈50 μL of the same buffer containing 100 U/mL of CIAP was applied to each section and incubated for ≧2 hr; samples then underwent a third round of ddTUNEL.

Preparation of Samples for Micrographic Imaging

For slides, after washing samples were then treated with an anti-fade DAPI mounting solution to stain DNA (Slowfade Gold®, Invitrogen), cover-slipped, and sealed using clear nail varnish.

For multiwall plates, after washing samples, the cells were incubated with 10 mM DAPI in PBST for 10 min, washed twice in PBST (10 min each), and 100 μL of PBST containing 0.02% sodium azide was added to prevent microbial growth.

Fpg-negative deoxyribitol samples were generated by incubation in 25 mM NaBH₄/70% methanol for 30 min. Fpg-negative dinitrophenylhydrazones samples were prepared by placing a drop of 15 mM DNP-hydrazine in 2.5 M HCl at room temperature for 30 min. After a one-hour incubation with 10% horse sera (Invitrogen), and subsequent washing, the DNP was imaged using a rabbit anti-DNP primary antibody 1:500 (Sigma) and donkey anti-goat FITC-labeled secondary antibody 1:500 (Invitrogen).

Labeling of H2A.X Histone and CD3ε Cell Marker

After incubation with 10% horse sera and washing, Ser139-phosphorylated H2A.X histone was imaged using an FITC-labeled monoclonal antibody (Biolegend, San Diego, Calif., USA) (1:500) following the methods of the manufacturer. Immune cell marker CD3ε was imaged using an FITC-labeled monoclonal antibody (Santa Cruz Biotechnologies, Santa Cruz, Calif.) (also at 1:500) as recommended by the manufacturer.

Rat Mammary Gland

Slides containing, 5-μm thick sections, fixed-paraffin embedded slices of rat mammary gland on Day 1 and Day 7 of involution, a model system of apoptosis, were purchased from Zyagen. Sudan Black was used to remove autofluorescence signals (0.3% Sudan Black/70% Ethanol) applied to the slide for 10 min, and then washed 8× with 0.1 M PBS.

U87 human glioblastoma cells were grown in DMEM with penicillin/streptomycin and 10% fetal bovine serum (FBS) (Invitrogen). Following 3-weeks' growth, 5 mL of cells were plated into slide chambers (Lab-Tek, Nalge Nunc International, Rochester, N.Y., USA) at 2×10⁵ cells per mL, and grown for 24 hrs at 37° C. in a 5% CO₂ incubator. The spent medium was removed, and 2 mL of fresh medium which contained; 1 mM H₂O₂, 300 μM Paraquat Sigma, 25 μM temozolomide carmustine, or 250 μM irinotecan (all chemotherapeutics obtained from Enzo Life Sciences International, Plymouth Meeting, Pa., USA), or an ethanol vehicle control was added. These concentrations were chosen as they represent the LD₅₀'s for these compounds on the selected cells (measured over a 24-hr incubation time). 24-hrs' later, cells were fixed with 2% PFA, then permeabilized in 0.1% Triton X-100® as described above.

Epifluorescence Microscopy

The signal was acquired using an Eclipse® TE2000-E fluorescent microscope (Nikon) equipped with a CoolSnap® ES digital camera system (RoperScientific) containing an CCD-1300-Y/HS1392×1040 imaging array cooled by a Peltier device. Images were recorded using NIS-Elements® software (Nikon) as described above.

Microscopic Calculations:

The pixel dimensions of the microscope/camera used has been previously calibrated by a representative of the manufacturer. At 100× magnification, each pixel element represented an interrogated area of 0.061×0.061 μm². At 40× magnification, the interrogated area per pixel is 0.162×0.162 μm².

For each 1 μm of sample depth, the volume is:

3.7×10⁻¹⁸ L (1×10⁻⁶×6.1×10⁻⁸×6.1×10⁻⁸ cm³) at 100× and is

2.62×10⁻¹⁷ L (1×10⁻⁶×1.62×10⁻⁷×1.62×10⁻⁷ cm³) at 40×.

Exemplary Anti-Mouse Cy3-Rabbit IgG Assay.

1. Obtain a pair of commercially-labeled antibodies (e.g., an FITC-mouse monoclonal primary and an anti-mouse Cy3-Rabbit secondary IgG).

2. Record spectrum of primary mouse IgG, and determine the ratio of FITC-to-antibody.

3. Dissolve mouse IgG in 15% gelatin, and then prepare a dilution standard curve.

4. Measure FITC in each sample using 300 μL in a 96-well plate.

5. Cast 200-μL blocks, cool, fix, dehydrate, wax, section and mount on slides that contain a series of 6-FITC calibration standards (e.g., a “Phantom Phlower”).

6. Rehydrate sections; treat with animal sera, wash, and then incubate with labeled secondary Cy3-Rabbit IgG antibody.

7. Measure the fluorescence levels of the FITC standards in each of the individual calibration standards (e.g., each “petal” of the “Phantom Phlower”).

8. Using this standard curve, calculate the levels of FITC in the FITC-Mouse monoclonal primary from the previously obtained FITC:IgG ratio, and calculate the levels of mouse IgG in each phantom.

9. Plot the level of Cy3 fluorescence against mouse IgG to give a standard curve of Cy3 vs. epitope concentration.

Results

The use of 3′ di-deoxyUTP as a TUNEL substrate in a ddTUNEL assay has been described and validated. Three ddUTP substrates were used for this study: a) biotin-16-ddUTP (Roche), which was visualized using FITC-avidin; b) biotin-16-ddUTP (Roche) which was visualized using Cy3-streptavidin; and c) 5-propargylamino-ddUTP labeled with PromoFluor-594® or PromoFluor-425® (PromoKine). These ddUTPs were chosen to compliment the excitation filters most commonly used in epifluorescence microscopy, and permit for the first time, quantification of the levels of DNA-OH 3′ ends in a histological specimen, tissue section, or in fixed cultured cells.

The ddTUNEL assay was shown to be superior to the traditional TUNEL assay, with respect to quantification of DNA —OH 3′ ends. In the classical assay, labeled dUTP (U-F) is the substrate and this is ligated to each —OH 3′ in the sample. However, each dUTP added to the DNA —OH 3′ also contains a TUNEL-positive —OH 3′ end, and therefore results in, intrinsically unquantifiable, polymeric labeling. In the present methods, ddUPT has been substituted for dUPT, to permit the stoichiometric addition of a single label at each, original, —OH 3′ end.

Fpg-ddTUNEL Assay; 2) Acylation or Oxidation

Two ways have been identified and validated in which oxidized bases can be detected (either by inference or directly), based upon their reactivity with sodium borohydride and 2,4-dinitrophenyl hydrazine (DNP-H) (Smith et al., 1998).

1) Reduction of DNA Carbonyls:

Incubation of the sample with sodium borohydride reduces all of the carbonyl groups in the sample, which therefore turns Fpg-ddTUNEL-positive oxidized DNA bases (oxo-DNA) into Fpg-ddTUNEL-negative bases (see e.g., FIG. 5).

2) Derivatization of DNA Carbonyls:

One can label all the carbonyl groups in a sample (including oxo-DNA bases) using DNP-H. The DNP-H forms DNP-hydrazone conjugates with biomolecules that have carbonyl groups, and the DNP-hydrazone can then be bound by an appropriately-labeled anti-DNP IgG. DNP-H will also react, of course, with Schiff bases, such as those that are generated from the reaction of paraformaldehyde with the primary amines of proteins (Ahmed et al., 2003). The background DNP-hydrazone level is low, however, as the products of the reaction of DNP-H with amine/formaldehyde-Schiff bases are soluble DNP-hydrazones that can be washed from the tissue (Smith et al., 1998).

Combined ddTUNEL, CIAP-ddTUNEL and Fpg-ddTUNEL Assay

FIG. 1B(1) and FIG. 1B(2) show a representative section of double stranded DNA featuring a variety of cuts, nicks and base modifications, all of which can be independently assayed. The DNA section begins with a Type II DNase blunt-ended cut, and along the 5′→3′ strand, there is a 8-oxo guanine, a DNase type I gap and finally a Type I DNase overhanging break with a —HO 3′ end. On the 3′→5′ strand, there are 3′ and 5′ phosphate ends, flanking a methylated guanine. After an initial round of ddTUNEL, using a fluorescently labeled ddUTP (F1), both of the —OH 3′ ends are labeled with a Red fluorophore [see e.g., FIG. 1B(3)]. The sample is then treated with CIAP, to generate a TUNEL-positive OH 3′ from the 3′-PO₄ [see, e.g., FIG. 1B(4)]. This newly-generated ddTUNEL-positive end can then be labeled using a biotinylated ddUPT [see, e.g., FIG. 1B(5)]. The sample was then treated with Fpg/CIAP, to generate a pair of TUNEL-positive —OH 3′ ends from both the oxo- and methyl-guanines [see, e.g., FIG. 1B(6)]. Finally, the newly-generated —OH 3′ ends were labeled using a second fluorescently-labeled ddUTP (F2) [see, e.g., FIG. 1B(7)].

Quantification of ddTUNEL.

In FIG. 2A, the images obtained from FITC-gelatin phantoms were then used to prepare a calibration curve, which is depicted in FIG. 2B. Images were deliberately chosen where sharp edges could be seen. The 6-μm thick FITC-gelatin phantom images (shown in FIG. 2A I to VIII), were recorded using a magnification of 40×, with an accumulation time of 100 msec. The final image (accumulated over 10 sec) showed the background levels of auto-fluorescence in un-conjugated gelatin. The obtained signal was proportional to concentration (see FIG. 2B), and the standard deviation for three different slides was 7.3%. The insert (FIG. 2C), shows the relationship between signal and accumulation time using three different 11.5-μM samples that were measured at a magnification of 100×. Again, the relationship was linear under the conditions employed. The relationship between signals at the two magnifications was in the order of 1.67:1 with regard to the illumination time required at a magnification of 100× to generate the same signal that was obtained at 40×.

The camera employed for these studies had a 1392×1040 pixel array. At 100× magnification each pixel represented an interrogated area of 0.06 μm², and at 40× magnification, each pixel represented an interrogated area of 0.16 μm². Thus, at a magnification of 100×, the volume interrogated by each pixel element, for each 1 μm of sample depth, is 3.6×10⁻¹⁸ L (1×10⁻⁶×6×10⁻⁸×6×10⁻⁸ cm³). At a solution concentration of 1 M, there were 2,167,920 molecules present in a 1-μm slice at 100×magnification. In 6-μm slices, there were 13 molecules per μM at 100× magnification, whereas under 40× magnification each pixel was interrogating 96 molecules per μM.

New Insights in Tissue Sculpting in Rat Mammary Gland.

Mammary involution in the rat is characterized by a massive loss of epithelial cells, due to apoptosis, and organ sculpting in this organ has been widely studied (Colitti and Farinacci, 2009; Bagheri-Yarmand et al., 2009; Lacher et al., 2003). One puzzle that remains in this tissues normal metabolic cell death is the nature of the clean-up crew. Do the characteristic apoptotic bodies that form consist of macrophages and lymphocytes or are they formed by cells of the breast it self and are cleared by macrophages and lymphocytes? (Tatarczuch et al., 1997; Kralj and Pipan, 1995; Walker et al., 1989.

In FIG. 3A, FIG. 3B, and FIG. 3C, the presence of ddTUNEL-positive nuclei and the formation of apoptotic bodies were shown at different magnifications in breast on Day 1. FIG. 3E, FIG. 3F, and FIG. 3G show the same tissue on Day 7 of involution. It can be seen that the apoptotic bodies contain both ddTUNEL- and Fpg-positive DNA. Moreover, this DNA is associated with histone γ-H2A.X (see FIG. 3D and FIG. 3H. It is important to note that the colors of the ddTUNEL probe were switched to demonstrate that the florescence of the apoptotic bodies was not due to cytoplasmic lipofuscin pigment, which has previously described in this tissue (Walker et al., 1989).

Signal Quantification of ddTUNEL.

In FIG. 3B a pair of ddTUNEL positive cells with a halo of digested oligonucleotides, around denuded DAPI nuclei, is highlighted by the dotted ellipse. In this case, FITC-avidin/biotinlylated-ddUTP was used in the ddTUNEL assay, and the FITC-Avidin had a ratio of FITC to protein of 1.03:1. The average signal correlated to a FITC phantom concentration of 24 μM, and thus it was possible to determine that in this pair of cells, there is approximately one —OH 3′ for every 640 basepairs. The cell nearby, arrowed, which has almost completed its apoptotic death, has a —OH 3′ for every 105 basepairs. If a cell were digested into the theoretical minimum-sized 180 to 200 bp fragments, there would be one —OH 3′ for every 95 basepairs (ignoring the contribution of nicks). The average number of cuts/nicks in the cells in the bottom right of FIG. 10B, which do not appear to be apoptotic, was approximately one free —OH 3′ for every 15,000 basepairs.

The role of lymphocytes in the cleanup of cell debris is shown in FIG. 10I and FIG. 10J. These sections were labeled with ddTUNEL, Fpg-ddTUNEL and with FITC-anti-CD3ε (a marker of lymphocytes). It could be seen that the lymphocytes were taking up DNA fragments (in the cytosol) and were associated with (but are not part of) the apoptotic bodies. To determine whether the DNA in the apoptotic bodies was oxidized or acylated, the sample was initially reduced, and it was found that the staining of the apoptotic bodies using Fpg-ddTUNEL was unaffected.

To discern if the DNA in apoptotic bodies was oxidized or modified by some other mechanism, sections were treated with DNP-H and then stained for DNA conjugates using an FITC-labeled anti-DNP antibody. FIG. 3K and FIG. 3L show the levels of carbonyls in breast tissue on Days 1 and 7, respectively. Carbonyl material was found in the apoptotic bodies, but it was not associated with DNA. Instead, it was discretely labeled protein that was found enshrouding the rounded, dice-like, DNA/histone material. Moreover, it appeared that much of this DNA had been methylated, even though it was known that the machinery of DNA methylation is normally down-regulated during apoptosis (see Vinken, 2010; Roos and Kaina, 2006). Instead, this suggested that nuclear-methylated DNA was packaged preferentially into apoptotic bodies using quite different methodologies (Andollo et al., 2005; Huck et al., 1999). Finally, inspection of the images from Day 1 and Day 7 showed that there was far more oxidative stress in the older tissue, and comparing the results in FIG. 3I and FIG. 3K with those of FIG. 3J and FIG. 3L strongly suggested that it was this oxidized material which was the main focus of the “clean-up crew”.

Effects of ROS and Chemotherapy on U87 Cells.

In FIG. 4A to FIG. 4K the formation of DNA breaks using the blue ddTUNEL and red Fpg-ddTUNEL; along with green DNA (YO-PRO-1), FIG. 4A to FIG. 4E or green γ-H2A.X (FITC IgG), FIG. 4F to FIG. 4K was shown. Absolute quantification of the levels of the ddTUNEL and Fpg-ddTUNEL in their respective cellular compartments was beyond the scope of this study, but data obtained from the sample shown in FIG. 4A to FIG. 4K was tabulated to demonstrate the approximate average concentration of the probes (assuming that each section was 6 μm thick) in the cells shown (Table 1):

TABLE 1
LEVELS OF DDTUNEL AND FPG-TUNEL
IN U87 CELLS FOLLOWING TREATMENT

| Treatment | Control | H₂O₂ | Paraquat | Carmustine | Temozolomide | Irinotecan |
|---|---|---|---|---|---|---|
| ddTUNEL | 3.7 | 9.4 | 37.2 | 22.8 | 8.7 | 7.4 |
| Fpg-ddTUNEL | 0.9 | 4.5 | 6.3 | 5.2 | 13.7 | 1.7 |

Shown are the approximate concentration (in μM) of the probes used for the ddTUNEL and Fpg-ddTUNEL in the cells depicted in FIG. 4A, assuming a (nominal) path length of 6 μm.

Control Cells.

There are many DNA nicks/breaks in the control cells and they appear green in FIG. 3A, but in FIG. 3G the background level of red blunt end breaks is more apparent, as is the presence of γ-H2A.X, which is mostly found in the cytosol. The absolute levels of all types of DNA damage in U87 cells is much higher than found in normal tissue samples, such as the rat breast tissue. Moreover, it was found that 2-5% of these cells are apoptotic at this stage of their growth cycle, and that there was considerable variation in the background levels of DNA fragmentation in different flasks. It was for this reason that a single slide/media/cells was used to grow the cells used to generate the paired images shown in FIG. 4A-FIG. 4K. For example, the images in FIG. 3A and FIG. 3G were from a single slide, and the cells used for different treatments were divided from one another using a hydrophobic liquid barrier pen (PAP Pen, Cat. No. 9804; Scientific Devices Laboratory).

H₂O₂.

The hydrogen peroxide treated cells (FIG. 4B and FIG. 4H) showed the formation of blebs which are stained for both histone and for ddTUNEL. The pattern of the distribution of DNA throughout the cells follows what was seen with the blunt-ended probe, ddTUNEL positive DNA is found in the cytosol and at very high levels in the small apoptotic blebs labels, but its levels are low within the nucleus and ddTUNEL tracks the distribution of γ-H2A.X closely. The red Fpg-ddTUNEL was also unevenly distributed. Oxidized DNA appeared to be rapidly exported from the nucleus and then concentrated up in the apoptotic vesicles. There also appeared to be some heterogeneity in the composition of these vesicles: some appeared to have high levels of γ-H2A.X, other appeared to have high levels of either ddTUNEL- or Fpg-ddTUNEL-positive DNA. The insert (FIG. 3G) is an enlargement of the indicated portion of these vesicles and suggests that vesicles are heterogeneous and that their formation may be a multi-pathway process.

Paraquat:

As was the case in previous studies, paraquat had a completely different death signature to H₂O₂. It was found that blunts were in the nucleus and overhangs were in the cytosol and in small vesicles, that were present in the cytosol, additionally there was no evidence of blebbing. ddTUNEL correlated with γ-H2A.X and was found elevated in the cytosol and highly concentrated in the small vesicles (FIG. 4C and FIG. 4H). There did seem to be evidence that oxidized DNA was preferentially exported out of the nucleus, and it was apparent from the bright white spots present in FIG. 4H that oxidized ddTUNEL-positive DNA and associated γ-H2A.X had been concentrated up into these vesicles.

Carmustine:

Treatment of cells with carmustine (FIG. 4D and FIG. 4I) induced six-fold increases in ddTUNEL and Fpg-ddTUNEL. In carmustine-treated cells, ddTUNEL ends were found in a 2:1 ratio in the cytosol compared with the nucleus. (In previous work, it was also shown that overhanging ends had a similar distribution). Moreover, the results indicated that blunt ended breaks and Fpg-ddTUNEL-positive DNA was overwhelmingly found in the nuclear compartment.

Temozolomide:

Cells treated with the methylating agent, temozolomide (FIG. 4E and FIG. 4J), gave the highest Fpg-ddTUNEL-positive result. It was apparent that this methylated DNA was exported into, and concentrated up in cytosolic vesicles—vesicles that are also γ-H2A.X and ddTUNEL rich. In comparing the ethylating agent, carmustine, and the methylating agent, temozolomide, the striking difference was the 10-fold difference in cytosolic γ-H2A.X levels in Carmustine treated cells, and these histones were not packaged into inclusion bodies. It was concluded that as methylated DNA which can be physiological, and larger acylation moieties are patho-physiological, is treated in a completely different fashion than is ethylated/acylated DNA.

Irinotecan:

The final pairing (FIG. 4F and FIG. 4L) showed the effect of irinotecan, on cell death. It had previously been demonstrated that irinotecan induces both overhang and blunt ended breaks, but with a ratio of 5:1 in favor of the former, and that blunt ends are localized in the nucleus and in apoptotic vesicles, and while these vesicles contain DNA, they have relatively low levels of γ-H2A.X. Here it was quite clear that ddTUNEL-rich DNA was intimately associated with γ-H2A.X and Fpg-ddTUNEL. One striking feature was that the DNA containing vesicles were again heterogeneous, containing either ddTUNEL with γ-H2A.X or Fpg-ddTUNEL rich DNA.

Oxidation or Acylation:

As noted above, the present methods were useful in differentiating between oxidation and acylation of DNA bases using reduction. This is demonstrated in FIG. 5. It was expected, a priori, that H₂O₂ would increase the levels of oxidized DNA, that carmustine would increase ethylation, and that irinotecan would not generate significant levels of either. To test this, half of a slide was incubated in ethanolic NaBH₄ to reduce all the oxidized bases. The results of the Fpg-ddTUNEL were then compared on all three incubations. As expected, only the H₂O₂-treated cells had an Fpg-ddTUNEL signal that was redox sensitive.

Summary:

In the present example, the venerable TUNEL assay (Gavrieli et al., 1992) has been improved, and a new methodology has been developed to quantify signals obtained from fluorescence microscopy, using the fluorescently-labeled gelatin tissue phantoms described above, to facilitate measurement of the absolute levels of Tdt accessible —OH 3′. The use of labeled ddUTP in a TUNEL-type assay has previously been used in a TUNEL/Tdt type assay where the ddUTP was labeled with digoxigenin and developed using a fluorescent anti-digoxigenin antibody (Anderson and Lee, 1997), an anti-digoxigenin-alkaline phosphatase conjugate (Abdelilah et al., 2001), and an anti-digoxigenin-peroxidase that was developed by use of diaminobenzidine/H₂O₂ (Ahlemeyer et al., 2001). However, in these cases there was never any attempt made to substitute ddUTP for dUTP to increase signal quantification.

Modification of DNA bases is a much-pursued strategy in the field of chemotherapy; cell death is included by using compounds that act as methylating/ethylating agents, nitrating/nitrosating agents, and as pro-oxidants that directly oxidize DNA bases or induce that induce oxidative stress and cause the formation of oxo-DNA indirectly. Such damage can be repaired in vivo by E. coli by Fpg, which removes a wide range of DNA lesions. Mammalian cells that have been transformed with E. coli Fpg have been shown to be more resistant to a range of toxic insults including the ethylating agent ThioTEPA (Gill et al., 1996; Kobune et al., 2001), ROS in the form of potassium bromate, H₂O₂ and Y-Rays (Frosina, 2001) and in the form of hyperoxia (Wu et al., 2002), the Carmustine-like ethylating agent bis(2-chloroethyl)-N-nitrosourea (Xu et al., 2001), and Carmustine (Ying-Hui et al., 2002).

It has been shown in the present example that the levels of both —OH 3′ and —PO₄ 3′ DNA ends can be assayed using ddTUNEL, and CIAP-ddTUNEL, respectively. Further, it has been demonstrated that E. coli Fpg can excise oxidized/acylated DNA bases in vitro, and that the —PO₄ 3′ ends generated after this action can be quantitative capped with fluorescently-labeled ddUTP, after treatment with a CIAP. These assays have been validated in an organ sculpting apoptotic model, the mammary gland (Walker et al., 1989), and in U87 cells treated either with oxidants, acylating agents, or with a topomerase I inhibitor.

Results demonstrated that it is now possible to differentiate between oxidized and acylated bases by treatment of the oxo-species by either reduction with borohydride or derivatization with dinitrophenyl hydrazine. This latter treatment allows one to interrogate the localization of ROS damage within a cell using an anti-DNP antibody.

The three techniques described herein, ddTUNEL, CIAP-ddTUNEL and Fpg-ddTUNEL, can be combined with the use of blunt and overhanging oligonucleotides, and with the use of tissue phantoms for signal calibration. By combining one or more of these techniques with the construction of tissue phantoms for signal calibration, researchers in cell physiology and patho-physiology are now able to interrogate cell death in significantly greater detail than was possible with previously-existing methodologies.

### Example 4

### Illustrative Products Exploiting Aspects of the Inventions

The standard, workhorse, epifluorescence microscope used in scientific research typically contains three default excitation/emission sets of optical filter blocks, typically called the DAPI-FITC-Texas Red Set (Table 2):

TABLE 2
NIKON TRIPLE BAND EXCITATION FILTER
COMBINATION SPECIFICATIONS

| Filter Set | Excitation | Polychromatic | Barrier | Imaging |
|---|---|---|---|---|
| Description | Filter (nm) | Mirror (nm) | Filter (nm) | Utility |
| DAPI | 395-410 | 445 | 450-470 | Violet Ex |
| | | | | Blue Em |
| FITC | 490-505 | 510 | 515-545 | Blue Ex Green |
| | | | | Em |
| Texas Red | 560-580 | 590 | 600-650 | Green Ex Red |
| | | | | Em |

This workhouse instrument was designed to generate images that have three components, a ‘blue’, a ‘green’ and a ‘red’ image. These have been optimized to allow the imaging of three probes.

The “DAPI channel,” 4′,6-diamidino-2-phenylindole is a fluorescent stain that binds strongly to A-T rich regions in DNA, and is by far the most widely-used conventional microscopy-based method for visualizing and imaging DNA. This channel, however, can also image DNA that is intercalated with one or more stains, including those of the Hoechst family of bis-benzimide-derived stains.

The “FITC channel” allows one to image amine-reactive fluorescein derivatives (such as FITC), including those conjugated to one or more biological probes (such as, for example, avidin/streptavidin, or combinations of labeled primary and secondary antibodies, etc.).

The “Texas Red channel” permits imaging of amine-reactive red fluorophores such as Texas Red (sulforhodamine 101 acid chloride), including those conjugated to one or more biological probes (such as, for example, avidin/streptavidin, or combinations of labeled primary and secondary antibodies, etc.).

In recent years, the use of FITC and Texas Red as labeling fluorophores has waned, as commercial vendors have introduced new fluorophores that have better intrinsic qualities than these first-generation fluorescent molecules. These new fluorophores have been developed to use the same filter sets present in existing epifluorescence microscopes, but have greatly improved properties over conventional compounds, including e.g., greater photostability, higher fluorescence intensities, etc. For example; FITC, which has excitation and emission spectrum peaks at 495 nm/521 nm, has been replaced by AlexaFluor® 488 (495/519 nm) (Invitrogen) and DyLight® 488 (493/518) (ThermoScientific, Waltham, Mass.), two dyes that have been tailored to have similar spectral characteristics to that of FITC, but with improved properties.

Using a fundamental microscope as an example (rather than more-sophisticated instruments which are capable of multilevel spectroscopy, etc.), this example outlines how tissue phantoms, ddTUNEL, and blunt-ended probes can be used in practical, real-world scenarios for quantitative determination of levels of proteins (e.g., antigens) and of different types of DNA damage in fixed, cells and tissues.

### Example 5

### Preparation of Microscope Slides Suitable for Commercial Vending for the Calibration of Fluorophores in Biological Samples

Phantom Flowers:

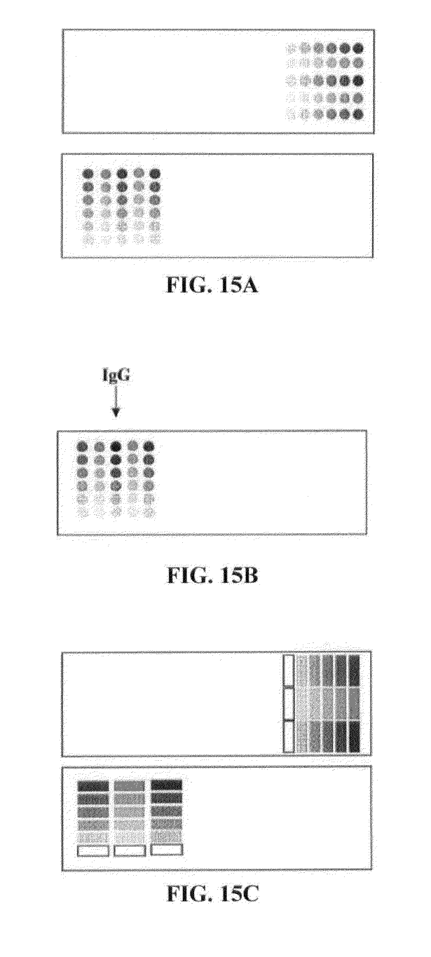

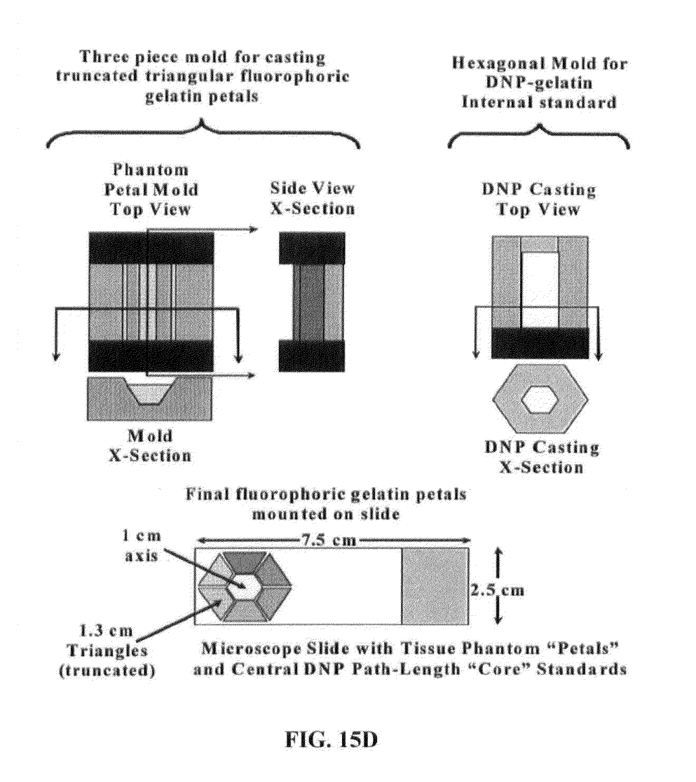

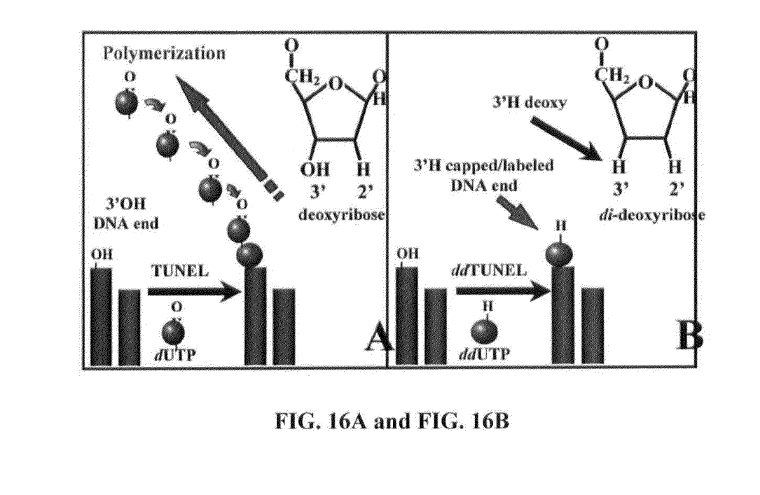

In FIG. 15A-FIG. 15D the inventors outlined how one may prepare a microscope slide containing a waxed, fixed, series of fluorophore-(such as FITC) labeled phantoms that also contain an internal, chromophore-based (such as DNP), path-length standard.

Preparation of Fluorophore Standard Fixed Casts:

Firstly, FITC-gelatin, in the range of 5 to 15%, is prepared. Using 15% gelatin as an example, and employing a maximum concentration of FITC on the order of 20 μM, four additional concentrations of FITC-gelatin may be prepared by dilution; for example a 15% gelatin solution that contains 20 μM conjugated FITC is initially prepared, then aliquots are diluted in 15% native gelatin to give rise to a concentration series of 20, 10, 5, 2.5, and 1 respectively. By using native gelatin alone in one sample, a 0 μM control is also preparable. The concentration of each of these solutions may be established by optical spectroscopy, e.g., using the known extinction coefficients (e.g., FITC has an ε₄₉₅ of 75,800 M⁻¹ cm⁻¹ at pH 8.5).

Each of the FITC-gelatin standards is warmed. Gelatin fluidity depends on the % of gelatin, but at 15% the solutions are preferably free-running at 45° C. The FITC-gelatin standards are then poured into a suitable form or mold, which, in the case of phantom “flowers” takes on the form of a truncated ‘V’ as shown illustratively in FIG. 15D. Of course, many other physical forms of the concentration standards are possible to arrange of the slide prior to commercial packaging (e.g., bars, dots, squares, wedges, etc.), the “flower” arrangement depicted herein, is a convenient, facile, and aesthetically pleasing arrangement of a set of standards on a given slide.

After pouring each of the standardized concentrations into their respective portions of a suitable mold, the mold and FITC-gelatin may be cooled (e.g., placed in a refrigerator) to approximately 4° C. to firm and set the gelatin standards. After cooling, the trench of the mold, which contains the FITC-gelatin samples, is fixed using cold PFA solution, 4° C. (preferably between about 2% and about 8% PFA), and then allowed to fully fix (preferably about 4 to about 48 hours).

### Example 6

### Preparation of Gelatin-Conjugated with a Chromophore For Sample Thickness Determination

An internal chromophore standard, such as using DNP or another suitable chromophore whose absorptivity is relatively high (e.g., the DNP-gelatin conjugate has an ε=17,530 M⁻¹ cm⁻¹ at 360 nm) may be operably linked to a gelatin solution (e.g., 15%) so that the absorbance peak of the final conjugated gelatin, in a section of a 5-μm pathlength, is ≧10× the signal-t-noise ratio of a typical spectrophotometer.

A typical laboratory spectrophotometer has a detection error of ±0.001 A at 360-312 nm; thus, a 2 mM DNP-gelatin internal standard will, when sliced to 5 μm thickness, generate a spectral peak at 360-312 nm of 0.01753 absorbance units, allowing the resolution of the phantom thickness of 5±0.3 μM.

Usage of chromophores with a higher extinction coefficient (for example, commercially-available cyanine dyes in amine-reactive forms, like Cy3, have extinction coefficients in the order of 250,000 M⁻¹ cm⁻¹) permit either improved resolution or the use of a lower concentration of conjugate; i.e., substituting Cy3 for DNP would allow the conjugated chromophore to be lowered from 2 mM to only 140 μM, while still providing a resolution of the final phantom thickness of 5±0.3 μm.

Preparation of a Chromophore Cast

In one illustrative embodiment, a readily-achievable, aesthetic, and practical form of the chromophore-gelatin standards includes one that is cast in a multiform (e.g., hexiform) mold, such as the one exemplified in FIG. 15D. After dispensing the appropriate volume of solution into the mold, the mold and the chromophore-gelatin contained therein can be placed in a refrigerator and cooled to about 4° C. to solidify the gelatin. After cooling, the trench of the mold, containing the FITC-gelatin can then be fixed (e.g., using cold PFA solution, 4° C.; between 2 and 8%), and then allowed to fully fix (e.g., 4-48 hours).

Cast Assembly, Dehydration, Waxing and Slicing.

The fixed, FITC-gelatin wedge-shaped casts (six in the case of a hexaform “flower petal” configuration) can then be conveniently arranged around one or more central fixed chromophore casts (the central part of the flower in the case of exemplary flower-petal arrangement). The form can then be sliced into appropriately-size (e.g., about 1 to about 5 mm) longitudinal sections, and then fitted into a suitable holder (e.g., a standard Tissue-Tek® embedding cassette [Sakura Finetek USA, Torrance, Calif.] provides a convenient holder for the illustrative hexagonal form depicted herein). The assembled phantom is then dehydrated and waxed in a suitable tissue processor (e.g., a Thermo-Shandon Pathcenter Tissue Processor (ThermoScientific Pathology, Waltham, Mass., USA) and then waxed using a suitable station (e.g., a Shandon Histocentre 3).

The waxed block can then be sliced using an instrument such as a Thermo Scientific Finesse 325 manual rotary microtome (Thermoscientific Pathology) and placed on universal standardized 2.5 by 7.5 cm microscope slides (See, e.g., FIG. 15A-FIG. 15D).

In this form, the phantoms can be conveniently stored (even at ambient storage conditions) for extended periods of time (e.g., many weeks to many months or more) without any significant loss of function. In illustrative studies, it was shown that a 15.5 μM Texas Red fixed, waxed, slide-mounted phantom stored at room temperature in a laboratory drawer for 12 months produced a signal equal to 96±6% of a freshly-prepared standard which generated a corresponding signal of 100±5%.

End-User Implementation

In the practice of the invention, an investigator will typically place a sample, fresh from a microtome, onto the center of the slide and allow the sample to dry. The entire is then subjected to conventional de-waxing, rehydration, and washing, after which the investigator would then label the sample of interest with at least a first FITC-bound detection probe. The resulting sample would then be cover-slipped and sealed, after which an investigator could then obtain an optical spectrum of the center of the ‘flower’ and record the absorbance of the chromophore. Knowing the extinction coefficient of that chromophore, the exact thickness of the phantom could then be calculated.

Next, the investigator images the sample of interest, preferably optimizing the instrument parameters towards that sample, and after completing that sample imaging, then records images of each of the FITC standards using the identical instrument settings employed for imaging the sample of interest.

By using the signals collected from each of the standards, the investigator would then construct a standard curve, converting the signals into known levels of fluorophore. From the standard curve, it would then be possible to absolutely, and definitively quantitate the amount of probe in the sample image, and thus determine the precise amount of the biological parameter of interest on a “per unit volume” or a “per cell” basis.

Phantom “Ladder Rungs” and Phantom “Polka Dots”

In the preceding description, it was shown how an internal chromophore could be used for an absolute thickness to be determined for a sample. However, using a measure of path-length is not necessary for all slides that are cut at the same time and from the same-waxed block. The thickness of such slices could be determined by the manufacturer, using a chromophoric block, and only part of the section, which contains fluorophore signal, would need to be mounted.

In FIG. 15B three different fluorophores are shown, with each comprising seven different concentrations that could also conveniently be applied to a slide, and thus form a commercial embodiment of the invention. The use of conventional geometric shapes, including rectangular- and/or cylindrically-shaped molds, would allow a manufacturer to make fluorophore phantom arrays; offered either as a standard production run (e.g., a slide with salmon sperm DNA, FITC and also Texas Red). Such slides would allow an investigator to construct three standard curves—one for each of the three channels of a typical fluorescence microscope, as shown schematically in FIG. 15B.

Because cylindrically-shaped standards would likely allow more of the slide to remain useful for sample placement, than if larger, square or rectangular standards would, the inventors contemplate that the use of cylinders of phantoms to produce “polka dots” on the slides would permit the mounting of standards of 5 or more known concentrations or 5 or more different fluorophores of a similar concentration, or a combination thereof, to produce ready-to-use microscope slides pre-formatted to contain a plurality of standards (see, e.g., FIG. 15C and FIG. 15D).

Phantom Epitopes and Proteins:

The use of antibodies and their visualization using a secondary antibody, labeled with a fluorophore, is a widely used practice in conventional microscopy. Typically, primary antibodies from one animal (for example, a mouse or a rabbit) are raised to a particular epitope, and then sold to researchers in purified form. Investigators then subsequently incubate one or more of these primary antibodies to a particular sample of interest, and after washing, then apply an antibody that was raised against antibodies to the primary species. (e.g., labeled goat IgG antibodies are most often used as a secondary antibody specific for a mouse primary antibody, although labeled donkey-anti-mouse, or donkey-anti-other animal antibodies are also commercially available and readily obtainable.

By incorporating known IgG standards into gelatin blocks, signals could be obtained from secondary labeled antibodies that would permit specific quantitation of the primary target. As an example, mixtures of IgGs from the most popular primary species (e.g., mouse, rabbit, and/or other species), could be prepared, e.g., in the range of about 5 μg/mL to about 5 pg/mL, which would permit a full range of antigens to be assayed on a given slide. A range of other proteins or peptides, including synthetic ones, could also be used to manufacture standard phantoms suitable for preparation into freshly prepared or commercially pre-prepared microscope slides.

### Example 7

### Exemplary Commercially-Vended Kits

The present example describes various illustrative commercially-vendable products, including diagnostic and quantitative kits, microscope slides containing pre-prepared quantitation/calibration standards, and a variety of compositions, and articles of manufacture that are now possible in view of the inventive compositions and methods disclosed herein.

3′-OH, 3′PO₄.

Abasic(apurinic/apyrimidinic sites) and total Fpg-sensitive sites. In the preceding examples, it has been shown how it is possible to quantify the levels of different types of DNA damage using biotinylated ddUTP. In a typical study an investigator would prepare four cell or tissue samples (e.g., Samples A, B, C & D), and from those samples, measure the levels of 3′-OH (A), 3′PO₄ (B), Abasic(apurinic/apyrimidinic sites) (C) and Fpg-sensitive sites (D), respectively, typically by using one channel; i.e., each type of DNA end would be visualized using a single type of labeled, biotin binding; such as Texas Red Avidin. However, different combinations and permutations of labeling; using differently labeled biotin binding proteins are, of course, possible.

ddTUNEL for 3′-OH.

All four samples are washed twice in terminal deoxynucleotidyl transferase (Tdt) reaction buffer; prepared by diluting a stock solution 1:5 of ddTUNEL buffer (125 mM Tris-HCl, 1 M sodium cacodylate, 1.25 mg/mL bovine serum albumin [BSA], pH 6.6) and a 25 mM cobalt chloride stock solution, 1:25. After washing some 50 μl of reaction buffer containing 20 units/mL of Tdt and 250 nM of labeled-ddUTP (Roche, Ind., USA) is applied to each of the samples, which are then incubated in a humidified box at room temperature for >2 hours or for 2 hours at 37° C.

In sample A, 3′-OH ends may be visualized by adding a fluorophore-labeled biotin binding protein, e.g., Texas Red avidin.

In samples B, C & D, all the biotinylated 3′ DNA ends are blocked with a native biotin binding solution (e.g., unlabeled avidin).

CIAP/ddTUNEL for 3′PO₄:

Samples B, C and D are washed twice in NEBuffer3 (New England BioLabs) and then incubated with 5-100 U/mL CIAP (Sigma) in NEBuffer3 for 1-2 hr at 25° C. They are then washed twice in Tdt reaction buffer and undergo a second round of second round of ddTUNEL: as above. After ddTUNEL the newly CIAP generated, 3′PO₄→3′OH, ends are all biotinylated.

In sample B, 3′-OH ends are visualized by adding a fluorophore labeled biotin binding protein, e.g., Texas Red avidin.

In samples C & D, all the biotinylated 3′ DNA ends were blocked with a native biotin binding solution, e.g., unlabeled avidin.

All Fpg-Sensitive Modified Bases:

Sample C is are washed twice in 10 mM HEPES, 10 mM NaCl, 2 mM EDTA and 0.1% BSA and then ≈50 μL of the same buffer containing 10-200 units/mL of Fpg (USB, Cleveland, Ohio, USA) is applied and incubated in a humidified box ≧1-5 hours. The sample is then incubated with 5-100 U/mL CIAP (Sigma) in NEBuffer3 for 1-2 hr at 25° C. Sample C was then washed twice in Tdt reaction buffer, and subjected to a third round of ddTUNEL: as described above.

In sample C, after dc/TUNEL the newly Fpg-CIAP-generated abasic sites and modified bases-→3′PO₄→3′OH, ends are all biotinylated and visualized by adding a fluorophore labeled biotin-binding protein, such as Texas Red avidin.

Only Fpg-Sensitive Modified Bases:

Sample D was incubated in 25 mM NaBH₄/70% methanol for 30 min and is then washed twice, for 30 min, in 10 mM HEPES, 10 mM NaCl, 2 mM EDTA and 0.1% BSA and then ≈50 μL of the same buffer containing 10-200 units/mL of Fpg is applied and incubated in a humidified box ≧1-5 hours. The sample is and then incubated with 5-100 U/mL CIAP (Sigma) in NEBuffer3 for 1-2 hr at 25° C. Sample D was then washed twice in Tdt reaction buffer, and then subjected to a third round of ddTUNEL, as above.

In Sample D, ddTUNEL the newly Fpg-CIAP generated, modified Base→3′PO₄→3′OH, ends are all biotinylated and visualized by adding a fluorophore labeled biotin binding protein; Texas Red avidin.

The difference in levels of fluorophore between samples C-D is equal to the level of Abasic sites in the samples. The reduction by borohydride of the apurinic/apyrimidinic sites from aldehyde to alcohol renders these sites un-reactive towards Fpg, and so they are not excised and converted into a biotinylated end.

### Example 8

### Commercially-Vended Kit for the Measurement of Blunt and Over-/Under-Hanging DNA Breaks Using a Biotinylated, Blunt-Ended Probe

In the previous examples, the inventors demonstrated how to measure the levels of blunt-ended DNA damage. It was shown that the ligation reaction between a Texas Red-labeled oligonucleotide was ligated to blunt ended DNA breaks with 100% efficiency, and with a 1:1 stoichiometry. Following this work, the inventors have introduced and tested a universal, blunt-ended probe, which is labeled with biotin. This probe, shown below, works in an identical manner to the Texas Red version, but has more flexibility, as it can be visualized using a vast range of commercially available biotin binding proteins that are fluorescently labeled, or labeled with probes that can be detected using some other type of spectroscopy; e.g., NANOGOLD® streptavidin can be used as a probe in immunoblotting, light microscopy or electron microscopy (Invitrogen).

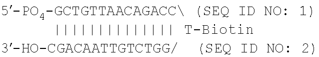

A Universal Probe for Measuring Both Blunt-Ended and Over-/Under-Hanging Ends.

Initially blunt ended DNA is labeled and then a biotin binding, fluorescently labeled protein is added (e.g., Avidin/Streptavidin). Then the over/under-hanging DNA breaks are converted to blunt ended breaks using modified, DNA Polymerase I/dNPT. After sculpting the ends, the newly-generated, blunt-ends are labeled in a second round of ligation, using the same biotinylated probe.

Blunt-End Ligation.

Slides are pre-incubated in the ligation buffer without the probe (66 mM-Tris HCl, pH 7.5, 5 mM MgCl₂, 0.1 mM dithioerythritol, 1 mM ATP, and 15% polyethylene glycol-8000) to ensure even saturation.

The buffer is aspirated, and the full ligation mix containing the ligation buffer with probe, 15-70 μg/μL (Sigma), and 0.1-1 U/μL T4 DNA ligase (New England BioLabs) is applied to the section, which is then incubated in a humidified box for 2-12 hours.

After washing, the probe can be visualized using fluorescently labeled avidin/streptavidin.

Sculpting Over and Under-Hanging DNA Ends.

The sample is washed and incubated in 1× NEBuffer2; 10 mM Tris-HCl, 50 mM NaCl, 10 mM MgCl₂, 1 mM dithiothreitol, pH 7.9 at room temperature for 30 min.

The sample is then incubated for >2 hours at room temperature in the same buffer containing 33 μM dNTPs and 50-500 Units/mL Klenow Fragment.

The Klenow Fragment is E. coli DNA polymerase I double mutant (D355A, E357A), that abolishes the 3′→5′ exonuclease activity but it retains the 5′→3′ polymerase activity.

In this case, the Klenow Fragment and dNTPs sculpt overhanging and under-hanging DNA ends into blunt ends (New England Biolabs).

Sculpted End: Over- and Under-Hanging-End Ligation.

After washing, the samples now undergo a second round of blunt end ligation with ligation mix containing the ligation buffer with probe, 15-70 μg/μL (Sigma), and 0.1-1 U/μL T4 DNA ligase. After washing, the biotinylated-probe can be visualized using a different fluorescently labeled avidin/streptavidin or other biotin labeled protein.

### Example 9

### Exemplary Quantitative Calculations Using Tissue Phantoms

This example shows illustrative calculations employing the tissue phantoms of the invention to determine the concentration of samples by fluorescence microscopy.

Calibration of DAPI Signal:

The calibration of the DAPI signal was performed by integrating the average signal per nuclei, to measure the total fluorescence per unit area and, using the (female) rat genome size of 5.353×10⁹ basepairs (bp), adjusting the look-up table (LUT) settings, so that in 8-bit color graphics, the maximum blue intensity of 255 units was equal to 200,000 bp.

Texas Red and FITC Phantoms Used for Calibration:

During the same 6 hour period all displayed images were recorded, the image of a Texas Red-gelatin tissue phantom; 5-μm thick; 15.5 μM Texas Red was recorded at the same magnification and time-base-100× and 250 ms. This phantom generated an average signal of 2517 (on a 0 to 4095 scale) after baseline subtraction. At 100× magnification, each pixel element had an interrogated area of 0.061 μm×0.061 μm and for each 1 μm of sample depth. The pixel volume at 100× was 3.7×10¹⁸ L; therefore, in a 5-μm phantom slice, there were ˜11.17 molecules/μM. Thus, 173 molecules/pixel of Texas Red gave a signal 2517, so each Texas Red molecule gave rise to a signal of 14.55 (on a 0 to 4095 scale).

An FITC-gelatin tissue phantom (5 μm thick, 12 μM FITC) was recorded at the same magnification and time-base as described above (100× and 250 ms). This phantom generated an average signal of 877 (in a 0-4095 scale), after baseline subtraction. Thus, 134 molecules/pixel of FITC gave a signal 877, so each Texas Red molecule gave rise to a signal of 6.54 (in a 0-4095 scale). The ratio of FITC to γ-H2A.X antibody was 1.8:1. Thus, a signal of 11.7 was equal to one antibody bound to a single histone at the site of a DNA break.

Recording of ddTUNEL (I), CIAP-ddTUNEL (II), Blunt (III) and Over- & Under-Hanging (IV) DNA Breaks:

Four tissue samples were labeled for 3′OH, 3′PO₄, blunt, and over-/under-hanging DNA ends, using the methods described herein. The biotinylated DNA ends were visualized using Texas Red Avidin, where the ratio of labeling was established to be 1.17 fluorophore per avidin tetramer. As one Texas Red molecules gives a signal of 14.55, each Texas Red labeled biotinylated DNA ended target gave rise to a signal of 17 (in a 0-4095 scale). The Texas Red signals were each recorded for 250 ms.

ddTUNEL 3′OH DNA:

In panel I, ddTUNEL labeled apoptotic tissue there were a pair of apoptotic cells. In both cells, assuming that the maximum thickness of the cell was 5 μm, and the peak signal was 1530, the Texas Red (nominal) concentration of 9.4 μM was thus equal to a biotin/3′OH concentration of 8 μM. The average signal (598) across the two cells was found across 124,320 pixels or 2×230 μm², giving 4,373,140 3′OH labels [(598×124320)/17)], which corresponded to one Tdt-reactive 3′OH end, for every 2,440 bp.

CIAP-ddTUNEL 3′PO₄ DNA:

Very few cells are labeled with CIAP-ddTUNEL, normally associated with necrotic cells death. In panel II, one such cell is labeled with CIAP-ddTUNEL giving average signal of 448, 2.7 μM Texas Red equal to 2.3 μM biotin/3′PO₄ ends, over 55,970 pixels. This represents 1,400,000 biotin molecules, or one, 3′PO₄-labeled end for each 3,630 bp.

Blunt Ends:

In panel III, blunt-ended, DNA-labeled apoptotic tissue there is a pair of apoptotic cells, each showing a peak at 8.5 μM biotin/blunt ended DNA breaks. Thus, there was a pair of blunt-ended breaks every 1,880 bp.

Over- & Under-Hanging Ends:

In panel IV, the tissue has been labeled for over-/under-hanging DNA ends. Thus, there was a pair of 0/U breaks every 3,300 bp.

The same images were also additionally imaged with γ-H2A.X antibody. This antibody is the “gold standard” for detecting the presence of DNA breaks in a nucleic acid molecule.

ddTUNEL 3′OH DNA:

One 3′OH end was present for every 2,440 bp, with the values for IgG being 1 in every 1,500 bp.

CIAP-ddTUNEL:

CIAP-ddTUNEL 3′PO₄ DNA analysis revealed one 3′PO₄ labeled end for each 2,800 bp, which was indicative of an atypical apoptotic cascade.

Blunt Ends:

There was a pair of blunt-ended breaks every 1,880 bp. Analyzing the green channel revealed one γ-H2A.X antibody for every 1,250 bp.

Over & Underhanging Ends:

There was a pair of O/U ends every 3,300 bp; analysis of the green channel revealed one γ-H2A.X antibody for every 2,200 bp.

### Example 10

### Thimerosal-Derived Ethylmercury is a Mitochondrial Toxin

Thimerosal, widely-used as a preservative, generates ethylmercury in aqueous solution. This example describes the toxicology of thimerosal in normal human astrocytes, with particular attention paid to mitochondrial function and the generation of specific oxidants. It was shown that ethylmercury not only inhibited mitochondrial respiration (leading to a drop in the steady-state membrane potential), but concurrent with these phenomena were increased formation of superoxide, hydrogen peroxide and Fenton/Haber-Weiss-generated hydroxyl radicals. These oxidants increased the levels of cellular aldehyde/ketones. Additionally, a five-fold increase in the levels of oxidant damaged mitochondrial DNA bases was observed as well as increased levels of mtDNA nicks and blunt-ended breaks. Highly damaged mitochondria were characterized by having very low membrane potentials, increased superoxide/hydrogen peroxide production, extensively damaged mtDNA and proteins. These mitochondria appeared to have undergone a permeability transition, an observation supported by the five-fold increase in Caspase-3 activity observed after Thimerosal treatment.

Thimerosal and Ethylmercury:

Thimerosal is a preservative that is widely used in medical products, including as a preservative in vaccines, immunoglobulin preparations, skin test antigens, antivenins, ophthalmic and nasal products, and tattoo inks, and is composed of 49.6 percent ethylmercury by weight (Suneja and Belsito, 2001). The widespread use of thimerosal exposes many to its potential toxic effects, especially in utero and in neonates. Here, the results of a series of experiments using cultured normal human astrocytes (NHA) exposed to thimerosal are shown in a study of the compound's effect on astrocyte mitochondria.

Oxidative Stress and Brain:

The brain utilizes 20% of the oxygen consumed by the body but constitutes only 2% of the body's mass (Clarke and Sokoloff, 1999). Some 5% of molecular oxygen consumption may arise from its reduction to superoxide (Korshunov et al., 1997). The majority of superoxide generated in cells comes from the reaction of molecular oxygen with flavin or quinone radicals, which are partly generated during respiration within complexes of the mitochondrial respiratory chain (Skulachev, 1999). The rate of reactive oxygen species (ROS) production increases steeply with increased mitochondrial membrane potential (Korshunov et al., 1997). Superoxide has a very short half-life in cells as it is rapidly dismutased (by either the cytosolic Cu—Zn superoxide dismutase (SOD) or the Mn-SOD in the mitochondrial matrix), producing molecular oxygen and hydrogen peroxide. Thus, generation of superoxide is always accompanied by hydrogen peroxide production, and so opens up the possibility of hydroxyl radical (HO.) generation via Fenton/Haber-Weiss chemistry (Sharpe et al., 2003). Fenton metals, including iron and copper, catalyze the production of HO. from superoxide/hydrogen peroxide and so the free, unchelated levels of transition metals inside cells is very low and normally all stored in an oxidized state. Normally, these metals are tightly bound to various metallochaperones, such as the ferric iron chelator ferritin.

Astrocytic Antioxidants in Humans:

Astrocytes are the major supporting cells of the brain and one of their key features is their ability to become ‘reactive’ towards infectious agents and use chemical warfare; upregulating iNOS to generate high levels of nitric oxide and NADPH oxidase to generate superoxide, hydrogen peroxide, peroxynitrite and other oxidative per-species (see Stewart et al., 2000 and references within). The types and levels of antioxidant enzymes of NHA are rather different from most other cell types and the levels of different enzymatic antioxidant enzyme change when NHA transition from ‘unreactive’ to ‘reactive’ states. In many cell types the main defense against peroxide stress are selenol containing enzymes including the glutathione peroxidases (GPx) and thioredoxin reductase (TrxR). GPx is not present in detectable levels in human ‘unreactive’ astrocytes in normal brain (Power. and Blumbergs, 2009) and it appears that GPx is only present in high levels in ‘reactive’ astrocytes (Ishida et al., 2006; Liddell et al., 2010). TrxR levels in normal human brain is also low, but is significantly elevated in the brains of Alzheimer's patients, especially at the site of amyloid plaques where ‘reactive’ astrocytes are present (Calabrese et al., 2006). It has been shown that in cultured NHA that TrxR expression is under tight regulation, with increases from very low basal levels, under the control of cytokines and growth factors (Mimura et al., 2011). Peroxiredoxins, including the mitochondrial Peroxiredoxin V, are an important class of peroxide/peroxynitrite detoxification enzymes that are sensitive to organomercury (Sarafian et al., 1997). Like the selenol based antioxidant enzymes, these thiol based antioxidant proteins are only found in very low levels in human astrocytes (Holley et al., 2007).

There is much evidence to suggest that catalase, rather than cysteine or selenocysteine based peroxidases, is the main enzymatic peroxidase in ‘unreactive’ NHA (Desagher et al., 1996). NHA also have high levels of reduced glutathione (GSH), capable of detoxifying peroxides via direct chemistry, and high levels of all three superoxide dismutases (Stewart et al., 2002; Rohrdanz et al., 2001). Catalase and the manganese superoxide dismutase are both up-regulated when astrocytes are subjected to oxidative stress (Desagher et al., 1996; Rohrdanz et al., 2001) In cell types where selenol/thiol containing peroxidases are the major enzymes that detoxify peroxides, organomercury toxicity tends to result from loss of antioxidant enzyme function coupled with an increase in the rate of oxidant production (Franco et al., 2009; Branco et al., 2012) There is a large literature examining the role of organomercury toxicity and the involvement of selenoenzymes TrxR and glutathione peroxidase GPx [see Branco et al. (2012) and references therein], however, these data may not apply to NHA, especially ‘unreactive’ NHA which appear not to make extensive use of these organomercury sensitive detoxification enzymes.

Localization of Organomercury-Induced Damage:

Ethylmercury is a lipophilic cation which can cross the blood-brain barrier (Barregard et al., 2011; Bragadin et al., 2002; Canty et al., 1984; and Yin et al., 2011). The octanol/water partition coefficients of methyl and ethylmercury are 1.4 to 1.8 (Canty et al., 1984; Mason et al., 1996), at intracellular pH and Cl⁻ concentration, thus both organomercury compounds predominately exist as lipophilic cations inside cells. Mitchell demonstrated that lipophilic cations accumulate inside mitochondria, in a Nernstian fashion, driven by the steady state membrane potential (Rich, 2008). Given that the typical mitochondrial membrane potential of astrocytes and neurons is between 140-170 mV (Clayton et al., 2005) one would, a priori, expect the concentration of these organomercury compounds within mitochondria to be approximately 1000× greater than the cytosolic concentration.

Ethylmercury and Mitochondria:

It was postulated that this compound was preferentially taken up into the mitochondria of NHA causing damage to the respiratory chain and subsequent ROS production. The damage of a cell's mitochondria leads to the activation of the apoptotic cascade and subsequent cell death (Korshunov et al., 1997; Skulachev, 1999; Rich, 2008; Abramov et al., 2010; Clayton et al., 2005; Dagda et al., 2009; Lossi et al., 2009; Mori et al., 2007; Shanker et al., 2005, and Whiteman et al., 2009).

This may be clinically relevant in the setting of a patient who harbors a known or unknown mitochondrial disorder. In the setting of a mitochondrial disorder, a specific mitochondrial toxin could be life altering or life threatening. These studies were performed to examine the effects of Thimerosal-derived ethylmercury on human astrocyte apoptosis by choosing a time course of cell examination after treatment that would highlight the early stages of apoptosis. The inventors' hypothesized that by examining the cells in an early phase (sixty minutes after ethylmercury dosing), compound's effect on the mitochondria and mitochondrial DNA (mtDNA) could be visualized.

Materials and Methods

Normal human astrocytes (NHA) were obtained from Lonza (Walkersville, Md., USA) and grown subject to their recommendations. NHA were grown to confluency in Astrocyte Cell Basal Medium supplemented with 3% FBS, Glutamine, Insulin, fhEGF, GA-1000 and ascorbic acid in 16-well Lab-Tek slide chambers (Nalge Nunc, Rochester, N.Y., USA), in a total volume of 240 μL.

Probes in Living Cells:

NHA were incubated for 1 hour with probes before fixation. Fixation in buffered paraformaldehyde (PFA) was performed in two stages. Firstly, a 50-4 aliquot of ice-cold 8% PFA was added to each well, then gently aspirated and the wells were twice washed with ice-cold 2% PFA and then allowed to completely fix at 4° C. After fixation, cells were washed twice in 1×PBS (Thermo Fisher Scientific, Rockford, Ill., USA). The tanks were then removed from the slides, the well area covered with Fluoromount-G (SouthernBiotech, Ill., USA), cover-slipped and sealed with nail varnish.

DNA was visualized using 1 μM Hoechst 33258 (Cat. No. H1398), mitochondrial membrane potential with 500 nM Mitotracker® Red (Dagda et al., 2009) (Cat. No. M22425), hydrogen peroxide using 5 μM H₂DCFAM (Whiteman et al., 2009; Setsukinai et al., 2003) (Cat. No. D399), mitochondrial superoxide generation with 5 μM MitoSOX™ Red (Abramov et al., 2010) (Cat. No. M36008), HO. was assayed using 5 μM hydroxyphenyl fluorescein (Setsukinai et al., 2003) (HPF) (Cat. No. H36004), with reagents obtained from Molecular Probes (Eugene, Oreg., USA).

Probes in Fixed Cells:

Fixed cells were permeabilized using 1×PBS with 0.1% Triton X-100®. Hydrazine reactive aldehyde/ketones were labeled using 225 μM Biotin-XX hydrazide (Hensley, 2009) (Cat. No. B2600) and visualized using Texas Red-Avidin (Cat. No. A820). The activity of Caspase-3 in fixed, 0.1% Triton-permeabilized cells was measured using the R110-EnzChek® Assay Kit (Molecular Probes, Cat. No. E13184), and incubating cells for 1 h at 37° C. (Scott et al., 2003).

DNA Labels:

The measurements and quantification of DNA 3′OH (ddTUNEL), oxidized DNA bases (Fpg-ddTUNEL) and blunt-ended breaks by use of the ddTUNEL and blunt-ended ligation were performed as described above (Baskin et al., 2010a; (Baskin et al., 2010b). Biotinylated ddUTP and biotinylated blunt-ended oligonucleotide probe was visualized using FITC-labeled avidin (Molecular Probes, Cat. No. 434411).

ddTUNEL: A Tdt reaction buffer was prepared daily diluting a stock solution 1:5 of TUNEL buffer (125 mM Tris-HCl, 1 M sodium cacodylate, 1.25 mg/mL BSA, pH 6.6) and a 25 mM cobalt chloride stock solution, 1:25. Each well was washed twice in this reaction buffer and then incubated with 50 μL of reaction buffer containing 20 units/mL of Tdt and 250 nM of Biotin-16-ddUTP (Roche, Ind., USA); labels were developed using FITC-labeled avidin (Cat. No. 434411).

CIAP-ddTUNEL:

Each sample, having previously undergone ddTUNEL, was washed and incubated with NEBuffer 3 for 30 min and then for at least 2 hrs with ≈50 μL of the same buffer containing 100 units/mL of calf intestinal alkaline phosphatases (Sigma) and the newly-generated, 3′PO₄→3′OH, ends.

Fpg-ddTUNEL Assay:

Following ddTUNEL/CIAP-ddTUNEL, capping all 3′OH/3′PO₄ ends with authentic, unlabeled, avidin, samples were washed twice in 10 mM HEPES, 10 mM NaCl, 2 mM EDTA and 0.1% BSA and then 50 μL of the same buffer containing 100 units/mL of formamidopyrimidine DNA glycosylase (Fpg) (USB, Cleveland, Ohio, USA) was applied to each of the wells, then incubated in a humidified box ≧2 hrs. Each sample was washed twice in 1×PBS (ThermoFisher Scientific), twice in NEBuffer 3 and ≈50 μL of the same buffer containing 100 units/mL of CIAP was applied to each section and incubated for ≧2 hrs; samples then underwent a third round of ddTUNEL and labeling with FITC-avidin.

Blunt-Ended DNA Breaks:

A biotinylated version of the blunt-ended oligonucleotide probe previously described (Baskin et al., 2010b) was employed in this study. The wells were pre-incubated in the ligation buffer without the probe (66 mM-Tris HCl, pH 7.5, 5 mM MgCl₂, 0.1 mM dithioerythritol, 1 mM ATP, and 15% polyethylene glycol-8000) to ensure even saturation. The buffer was aspirated, and the full ligation mix containing the ligation buffer with probe (35 μg/μL) and 0.5 U/μL T4 DNA ligase (New England BioLabs) was applied to the sections, which were then incubated in a humidified box overnight.

Thimerosal:

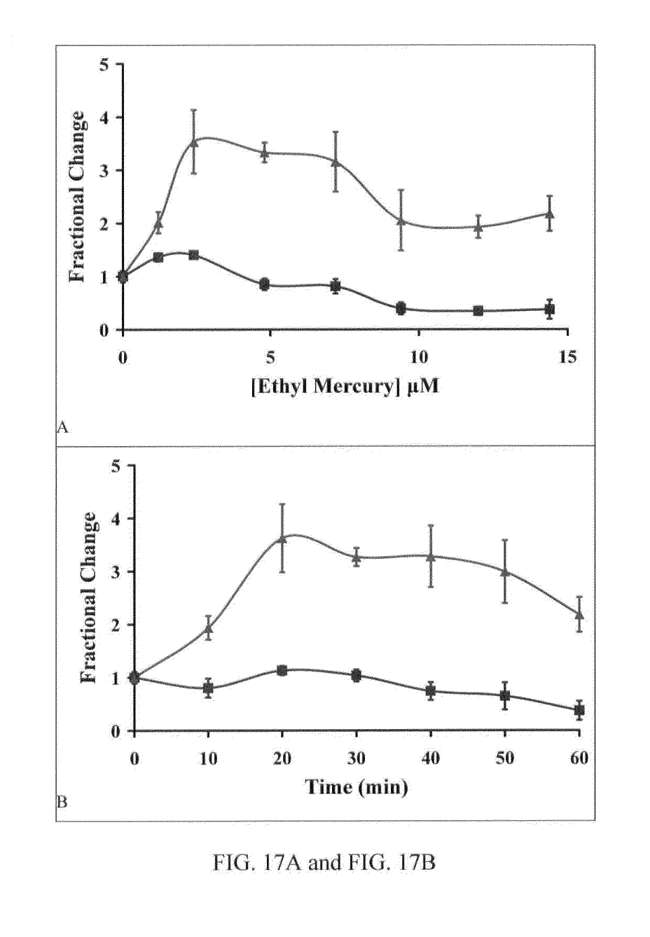

Thimerosal≧97% (HPLC) and all unspecified reagents were obtained from Sigma-Aldrich, unless otherwise specified. Thimerosal solutions were prepared in 1×PBS (ThermoFisher Scientific) to a maximum concentration of 360 nM and 10 μL were added to the 240 μL astrocytic volume. 3% FCS was present in the NHA medium throughout the time-course. To generate the time-course shown in FIG. 17A and FIG. 17B, NHA were exposed to Mitotracker, H₂DCFAM and Hoechst at t=0. Additions of 10-μL aliquots were added at 10-min intervals, to different wells in sequence, so that all the cells had the same length of exposure to the reporters, but different temporal exposure to thimerosal.

Epifluorescence Microscopy:

The signal was acquired using an Eclipse™ TE2000-E fluorescent microscope (Nikon) equipped with a CoolSnap® ES digital camera system (Roper Scientific) containing a CCD-1300-Y/HS1392×1040 imaging array cooled by a Peltier device. Images were recorded using and analyzed using NIS-Elements® software (Nikon) and images were stored as both .jpeg200 and .jpg files.

Results

Mitochondrial Membrane Potential and ROS Generation Following Thimerosal Incubation:

In this example, the effect of ethylmercury on the fluorescence levels of the three reporters was investigated in two ways. The concentration dependence of ethylmercury towards NHA was studied by adding to 0-14.4 μM Thimerosal to the cell media at t=0. In addition, the temporal changes caused by the addition of 14.4 μM thimerosal at t=0, 10, 20, 30, 40 and 50 min before fixation at 60 min were investigated. The center field of three independent wells were imaged, at each time point or concentration, and the fluorescence levels of the three reporters collected of an average of 44±18 individual astrocytes per center field. In FIG. 17A and FIG. 17B, changes in the levels of MT and ROS (via DCF formation) are shown as a function of thimerosal concentration (FIG. 17A), and of changes induced by incubation with 14.4 μM thimerosal over time (FIG. 17B). It can be seen that low concentrations of ethylmercury caused an increase in both signals. The finding that ethylmercury increases ROS generation was not unexpected, given the known effects this agent has in disrupting cellular thiol/glutathione based antioxidant defenses (Bragadin et al., 2002; Yin et al., 2011; and Shanker et al., 2005). The hyper-polarization of mitochondrial membrane potential was unexpected, given that depolarization of mitochondria has been observed in most cell types prior to apoptosis. At higher concentrations (>7.2 μM thimerosal) a loss of mitochondrial signal and of DCF was observed. This loss of signal, when comparing >7.2 μM with <7.2 μM thimerosal, correlates well with changes in cell morphology; cell shrinkage and the formation of a ruffled plasma membrane and blebs. In the time-course of ethylmercury-induced changes shown in FIG. 17B, it was observed that the generation of ROS species was an early event, and that there was an increase in ROS generation prior to changes in mitochondrial membrane potential. The levels of cellular DCF began to fall at >40 min, and this drop in the levels of the cellular ROS reporter also corresponded to the observation of cell shrinkage and the formation of cytoplasmic blebs.

Co-Localization of Mitotracker and ROS:

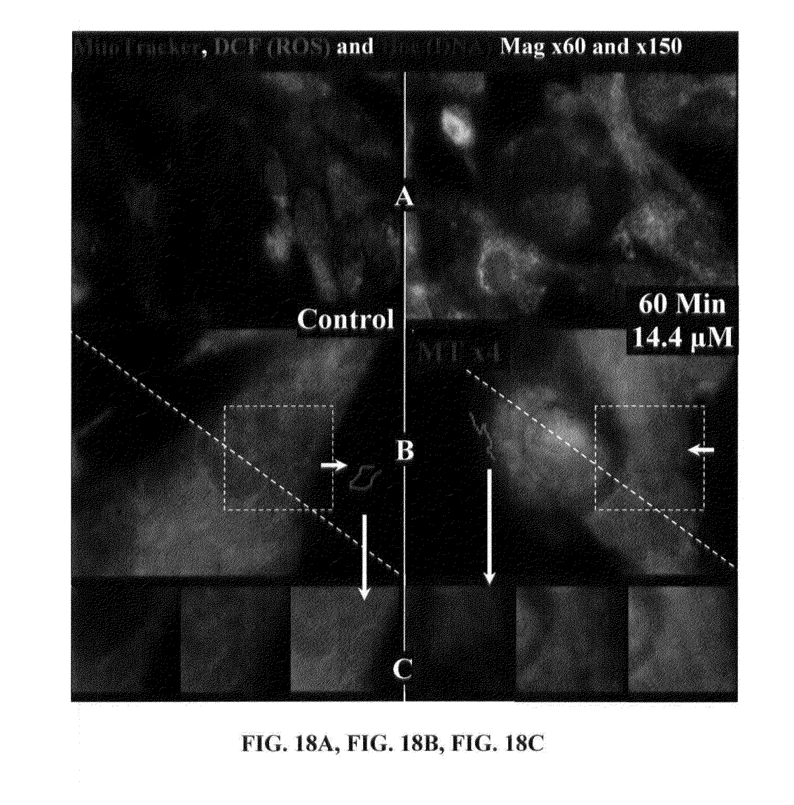

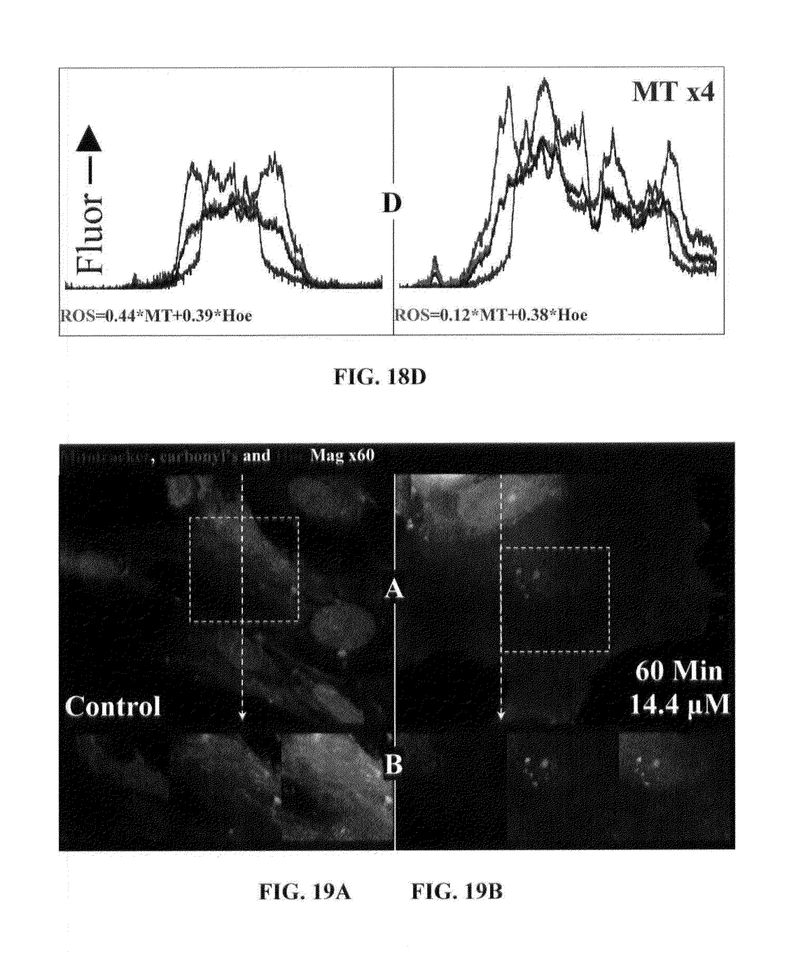

In FIG. 18A and FIG. 18B, it was shown that the co-localization of mitochondrial and ROS signals in high-resolution images of control NHA treated for 60 min with 14.4 μM thimerosal. In FIG. 18A, upper panels, the Mitotracker (red), ROS induced DCF (green), and nuclear Hoechst staining (blue) of NHA taken at magnifications of ×60 in the absence (left) and presence (right) of 14.4 μM thimerosal are shown. The fluorescence levels of all three panels were matched in the two images, so that the color levels absolutely reflected signal levels and showed that thimerosal caused an approximately 50% drop in mitochondrial membrane potential, and a two-fold increase in ROS. It was clear that the majority of mitochondria in the cells were in a vermiform network, and that there appeared to be a strong co-localization of the mitochondrial and ROS signals. In FIG. 18B the images of control and treated cells obtained at 150× magnification are shown. Here, the red mitochondrial signals were multiplied by a factor of four in the 14.4 μM thimerosal-treated astrocytes to allow visual identification of the distribution of the mitochondria within these cells. The three treated cells shown were reasonably representative of the population, with the central cell being shrunken, and with a highly-distorted nucleus. The square outlines are areas of the cells where individual Mitotracker and ROS images are presented, and the overlaid images of these fluorophores of these chosen areas are shown in FIG. 18C. These images clearly showed that an orange-colored ‘horseshoe-shaped’ signal in the control cell (see FIG. 18B) consisted of a network of mitochondria, and that this mitochondrial network was mirrored in the DCF, ROS image. The correlation of mitochondrial signaling in the treated cells was also indicated, and in one of the treated cells, a ‘lightening bolt’-shaped mitochondrial network was observed. One can note that this ‘lightening bolt’ feature consisted of a mitotracker-positive network of mitochondria and DCF signals.

Both images in FIG. 18B have a diagonal line running from top-left to bottom-right. The bottom panel, FIG. 18D, shows the intensity profile of MT, DCF and Hoechst along these two lines (with the MT signal 4× in the thimerosal-treated image). The red lines correspond to the fluorescence signal of MT, the blue lines to Hoechst- and the green lines to ROS-generated DCF. In both plots, there was an additional black line, which matched the line shape and amplitude to the DCF signal. This black line was a fit to the ROS signal, based on the amplitudinal changes of MT and Hoechst. In the control panel, the ROS signal was best simulated by 0.44 multiplied by the MT signal and 0.39 multiplied by the Hoechst signal. In the thimerosal-treated cells, the relationship between Hoechst staining and DCF levels was within 3% of that in the control cells modeled at 0.38 multiplied by Hoechst fluorescence. However, the fit with MT labeling was strikingly different, with the best simulation generating a value of 0.117 for the ratio of actual MT signal to ROS. Cross-correlation of the simulated fit was compared to the actual DCF signal, and it was found that the slopes were 1±0.01 in both cases, and further that the R² values were greater than 0.99 in both controls and treated cells. Thus, thimerosal-treated astrocytic mitochondria were generating four times the amount of ROS as the control mitochondria, but the steady-state generation of ROS in areas with no mitochondria, especially the nucleus, was unchanged.

ROS Damage and Mitochondrial Membrane Potential:

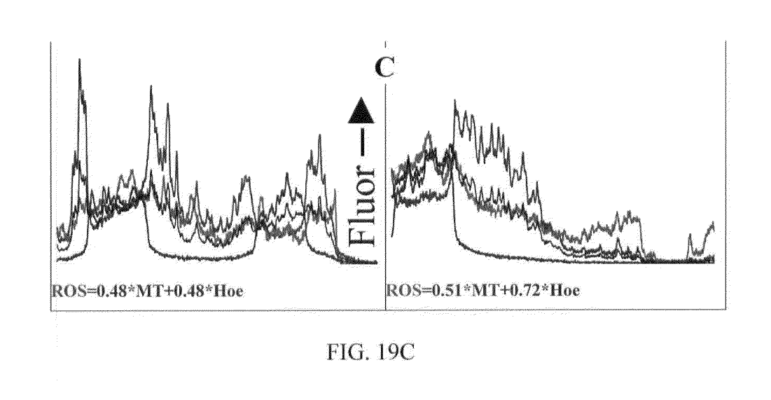

In FIG. 19A-FIG. 19C it was shown that damage from ROS, in the form of aldehyde/ketones (carbonyls) was also co-localized with mitochondrial membrane potential, and that more carbonyls were present in thimerosal-treated NHA. FIG. 19A shows control and 14.4 μM thimerosal prepared using MT and Hoechst, then treated with Biotin-XX hydrazide carbonyl labeling, which was visualized using FITC-avidin. FIG. 19A shows control/thimerosal treated cells where all three fluorophores have the same scale. The images of a large ethylmercury treated cell were selected (although somewhat unrepresentative of the population size distribution), as larger cells allow easier discrimination of the mitochondrial network. What was noticeable is that there was an increase in green ROS-damaged cell contents as a function of distance from the nucleus in both images. The two boxes in FIG. 19A show areas highlighting the correlation between MT and carbonyl signals. These areas are shown as single images of MT and carbonyls, and as a merged image in FIG. 19B. In the control cells, it is clear that the network of mitochondria is co-localized with some networks of carbonyls, but there are some well-defined structural networks, which show evidence of oxidative stress that do not correlate with mitochondria. A similar pattern was observed in thimerosal-treated astrocytes; there are quite clearly networks of mitochondria, carbonyls, and structures that contain evidence of ROS damage, but without polarized mitochondria. The two vertical lines in FIG. 19A indicate the position the fluorophores were interrogated to generate the fluorophore profiles shown in FIG. 19C. Again, the three colors represent different fluorophores, MT (red), carbonyls (green) and Hoechst (blue), and the black line is a simulation of the levels of ROS damage generated by combining fractions of the MT and Hoechst signals. In both samples, the simulation is a poor match for the actual ROS-induced signal, but control cells give a much poorer fit than do thimerosal-treated astrocytes. Cross-correlations of ROS vs. our simulation of carbonyl levels generate slopes of 0.75 and 1.1 for controls and ethylmercury treated cells, and prove R² values of only 0.68 and of 0.86, respectively. Therefore, although it was observed that generation of ROS is highly localized to mitochondria the cellular distribution of markers of ROS damage is poorly localized with mitochondria.

It therefore appears that proteins suffering ROS damage, and so having carbonyls, are transported from the regions where they have been damaged. Vesicles containing high levels of carbonyls are present in both the controls and treated cells, however, in the cells that have been incubated with ethylmercury a large number of small, <500 nm, clumps of oxidized material were observed. A possible origin of this material is that it represents flocculated damaged mitochondria that are unable to maintain a membrane potential, such as that which occurs following the mitochondrial permeability transition. This clumping of mitochondria has previously been described during the early stages of apoptosis and has shown to be a result of the activation of the BH3 domain of BAX (Fitch et al., 2000).

Co-Localization of ROS Damage and mtDNA Damage; Thimerosal Attacks mtDNA:

It was initially postulated that cationic, lipophilic ethylmercury should partition into the mitochondrial matrix, and that accumulation should be driven by the mitochondrial membrane potential. As mtDNA is restricted to the mitochondrial matrix, an increase in the steady state of ROS in this compartment should act as a reporter of this oxidative stress. The presence of 3′OH DNA breaks or of Fpg-labile modified DNA bases was examined using ddTUNEL and Fpg-ddTUNEL (Baskin et al., 2010a), and additional aldehyde/ketones (carbonyls) using Biotin-XX hydrazide. Cells grown in the absence or presence of 14.4 μM thimerosal were labeled for the presence of 3′OH DNA nicks (ddTUNEL) or for oxidized/acylated DNA bases (Fpg-ddTUNEL) using biotinylated ddUTP. These DNA ends were visualized using FITC-avidin and carbonyls with Texas Red-avidin; nuclei were again labeled with Hoechst. The signals in the recorded images showed blue nuclei, red carbonyls and, green 3′OH DNA ends or green Fpg-labile DNA bases/apurinic or apyrimidinic sites, reflecting the levels of fluorophore in each of the images. Taken together, the results indicated that at one hour of incubation full-blown apoptosis was not observed, which is characterized by nuclear DNA fragmentation and are indeed observing the early phases of cell death. Moreover, there was a clear co-localization of DNA damage and the presence of carbonyls. The damaged DNA was cytosolic, not nuclear, suggesting mitochondrial DNA damage. By demonstrating a co-localization of mitochondrial DNA damage and ROS in the cytosol of the NHAs, it was shown that the mitochondria may be responsible for the generation of ROS in the presence of ethylmercury and are the primary inducers of the apoptotic cascade.

The Identity of the Oxidant Produced by Ethylmercury in Mitochondria:

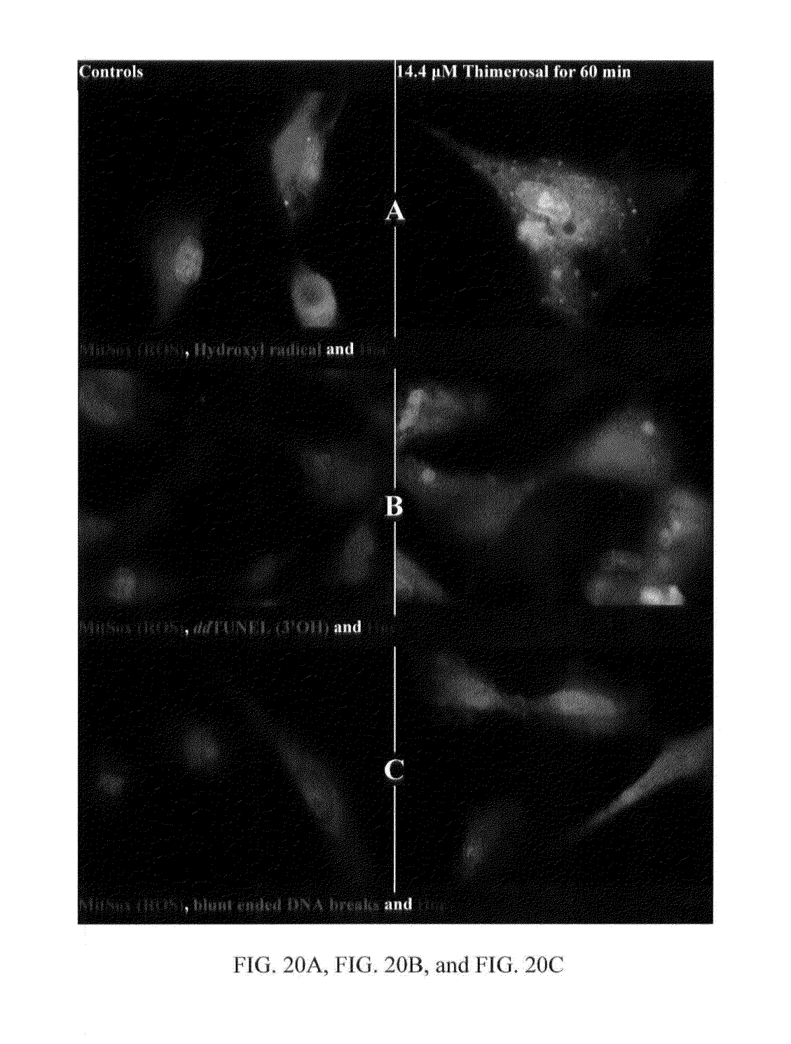

The production of ROS was measured using the mitochondrial superoxide probe MitoSox™, and additionally measured HO. via hydroxyphenyl fluorescein (HPF), 3′OH DNA ends with ddTUNEL and blunt-ended DNA breaks in NHA incubated for 1 hr with 14.4 μM thimerosal. FIG. 20A shows that reporters for both superoxide and HO. were highly co-localized, giving R² values of >0.98, and thus superoxide generation leads to Fenton/Haber-Weiss chemistry inside mitochondria. Treatment of NHA with ethylmercury led to a 90% increase in superoxide generation per cell, even though under the same conditions a 50% drop in mitochondrial membrane potential was observed. Deconvolution of superoxide and HO. signals show that the presence of ethylmercury results in 60% more HO. generation per superoxide. FIG. 20B and FIG. 20C show that superoxide generation correlates with non-nuclear, thus mtDNA damage in the form of ddTUNEL 3′OH DNA ends and also of highly damaging blunt ended DNA breaks (Baskin et al., 2010b). The scaling of the two green channels in FIG. 20B and FIG. 20C differ by a factor of four, and this indicates that there are, on average, nine times as many 3′OH ends as there are DNA breaks in the control mitochondria.

Global Changes in Mitochondrial Function and Cellular Damage to NHA Resulting from Exposure to Ethylmercury:

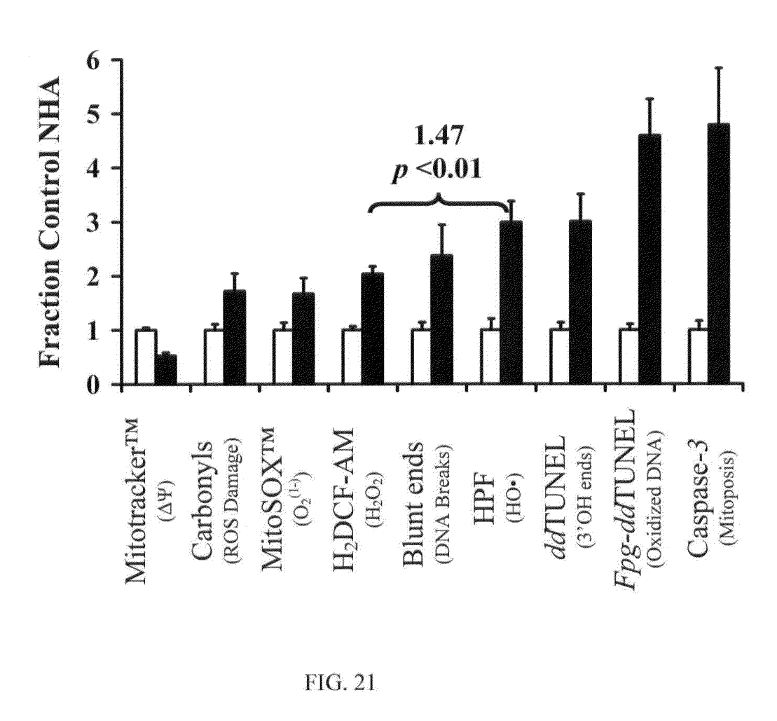

In FIG. 21 a bar plot is presented that shows the summarized changes observed in NHA following a 1-hr exposure to 14.4 μM thimerosal. All plots represent the average signal levels with respect to control cells. Five images were taken from three parallel experiments with an average of 44±18 individual astrocytes per visual field, and the error bars represent the SD of the population.

Ethylmercury causes a 50% collapse in membrane potential in astrocytes at 1 hour. Accompanying this collapse in membrane potential, a significant increase was observed in the levels of various ROS. The internal mitochondrial steady state level of superoxide increases by ≈70% in treated cells and is matched by an increase in cellular hydrazine reactive carbonyls. Using H₂DCF-AM, a 200% increase was observed in steady-state production of reactive oxidants, which from deconvolution is known to be mitochondrially-generated (FIG. 18A-FIG. 18D). Mitochondrial DNA, and not nuclear DNA is far more vulnerable to ethylmercury induced damage. A 240% increase was observed in the levels of mitochondrial DNA breaks, a 300% increase in 3′OH DNA nicks and a 460% increase in the levels of oxidized bases/apurinic or apyrimidinic sites. As mtDNA is localized within the mitochondrial matrix, it follows that this was the main site of ROS generation. The 300% increase in HO. was ≈80% greater than the increase in superoxide generation. As Fenton/Haber-Weiss chemistry is the primary generator of HO. in biological systems, this finding suggested that ethylmercury was also increasing the levels of Fenton metals (such as iron and copper), inside astrocyte mitochondria. The final pair of bars illustrate the change in the levels of Caspase-3 activity, measured by examining the cleavage of a Z-DEVD-R110 substrate. A five-fold increase in Caspase-3 activity was also observed, indicating that this pathway had also been activated in thimerosal-treated cells.

It was found that treatment of NHA with ethylmercury caused an increase in mitochondrial superoxide generation, however, the increase in superoxide generation was identical to the increase in the levels of protein carbonyls (FIG. 21). H₂O₂-induced formation of dichlorofluorescein from H₂DCF-AM was only approximately 20% greater than superoxide/carbonyl formation, which suggested that the loss of peroxidase function was not a feature of NHA ethylmercury toxicity. This was consistent with the effect of methylmercury on HeLa cells, where mitochondrial matrix generation of superoxide was implicated as the most damaging ROS (Naganuma et al., 1998). HeLa cells can be protected from methylmercury toxicity by upregulating mitochondrial Mn-SOD, but not cytosolic Cu/Zn-SOD, GPx or catalase.

The majority of protein carbonyls in controls and in ethylmercury-treated NHA were also co-localized with mitochondria (FIG. 18A-FIG. 18D). The peroxides measured via H₂DCF-AM and protein carbonyls, are derived from mitochondrial ROS generation, as shown by colocalization of signals with the specific mitochondrial superoxide probe, MitoSox (FIG. 20A-FIG. 20C). These findings were in broad agreement with the known generation of ROS, on either side of the inner mitochondrial membrane, in normal mitochondria (St-Pierre et al., 2002) and effects of methylmercury on rodent astrocytes observed by Shanker and co-workers (Whiteman et al., 2009), as they too identified that mitochondria are the main production sites of increased superoxide generation.

In addition to measuring peroxide/superoxide generation, also observed was the formation of HO., using the specific probe, HPR and using the Fpg-ddTUNEL assay which measures oxidized DNA bases. The conversion of guanine to 8-hydroxyguanine and 8-hydroxyguanine to more oxidized DNA hydantoin lesions, spiroiminodihydantoin and guanidinohydantoin, is generally believed to be due to HO. or to Fenton's reagent (oxy-ferry; Fe^{(IV)}═O⁽²⁻⁾) and oxy-cupryl Cu^{(III)}═O⁽²⁻⁾) (White et al., 2003).

8-hydroxyguanine, spiroiminodihydantoin and guanidinohydantoin are substrates from the Fpg-ddTUNEL assay (Baskin et al., 2010a; Krishnamurthy et al., 2007). It was demonstrated that whilst the levels of damaged nuclear DNA and mtDNA are very low in untreated cells, ethlymercury induces a large increase in oxidized mtDNA lesions. The highest levels of damaged mtDNA and protein carbonyls occur in structures that appear to be flocculated mitochondria. These grainy, oxidized, structures are not present as bright grains when viewed using Mitotracker, when carbonyl rich grains can be identified. These same vermiform structures were also identified in treated cells labeled with specific probes for both superoxide and HO. (see FIG. 20A-FIG. 20C). However, although an increase in the levels of cytosolic (and hence, mitochondrial) blunt-ended breaks and nicks were observed, very high levels of DNA breaks were not present in granular form. Thus, these flocculated mitochondria represent a dead-end mitochondrial state and given the close correlation between Fpg-ddTUNEL and Caspase-3 upregulation (FIG. 21), it was reasonable to conclude these are mitochondria that have undergone the permeability transition (Bragadin et al., 2002), resulting in the release of pro-apoptotic proteins like cytochrome c and DIABLO from the inter-membrane space, mitoposis, and the initiation of the Caspase-3 apoptotic cascade.

The Mechanism of Superoxide, Peroxide and HO. Generation in NHA:

It has long been known that organomercury reacts with iron sulfur centers (Arakawa and Kimua, 1980); indeed methylmercury has been used as an aid to identify mercury adducts in iron-sulfur protein crystal structures for decades. The reaction of organomercury with iron sulfur centers in proteins such as aconitase results in loss of enzymatic function, the formation organomercury thioether adducts, and exposure to the bulks aqueous phase to redox active iron or release of free iron. It has been shown that, in mouse brain, the mitochondrial iron-sulfur complex rich enzyme NADH/Quinone oxidoreductase (Complex I) is highly sensitive to methylmercury (Glaser et al., 2010). In a study by LeBel, Ali and Bondy it was found that methylmercury neurotoxicity was partially iron mediated (LeBel et al., 1992). The potent iron-chelator, deferoxamine, protected rat cerebellum from ROS following an injection of methylmercury. Iron chelation also protected neurons from ROS following in vitro exposure to methylmercury, but there was no evidence of deferoxamine-mercurial complex formation (King, 1964). Methylmercury treatment of isolated mitochondria, from the cerebrum, the cerebellum and from liver, causes an inhibition of respiration and increased superoxide/hydrogen peroxide formation (Mori et al., 2007), mostly via damage to succinate dehydrogenase. The three iron-sulfur centers of succinate dehydrogenase, on the matrix side of the inner mitochondrial membrane, are the likely site of inhibition and possible iron release given that these clusters are sulfide/iron labile towards the thiophilic reagent, p-chloromercuribenzoate (King, 1964).

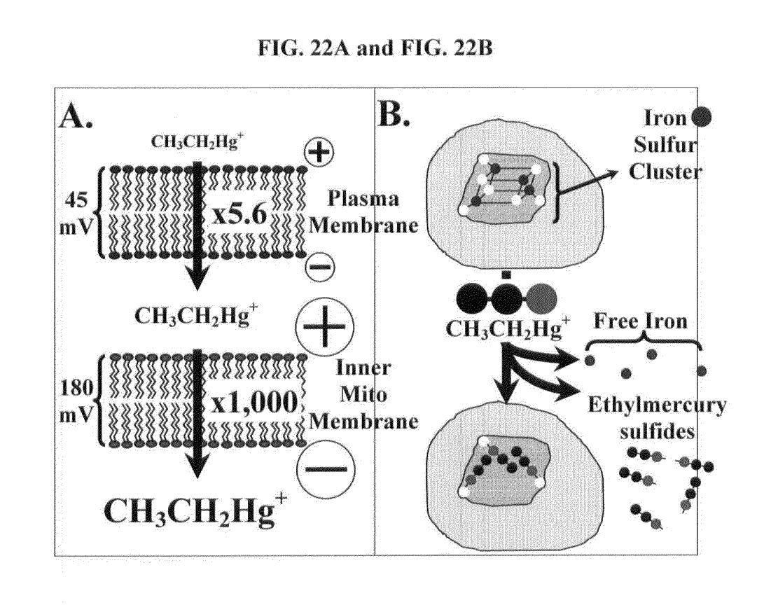

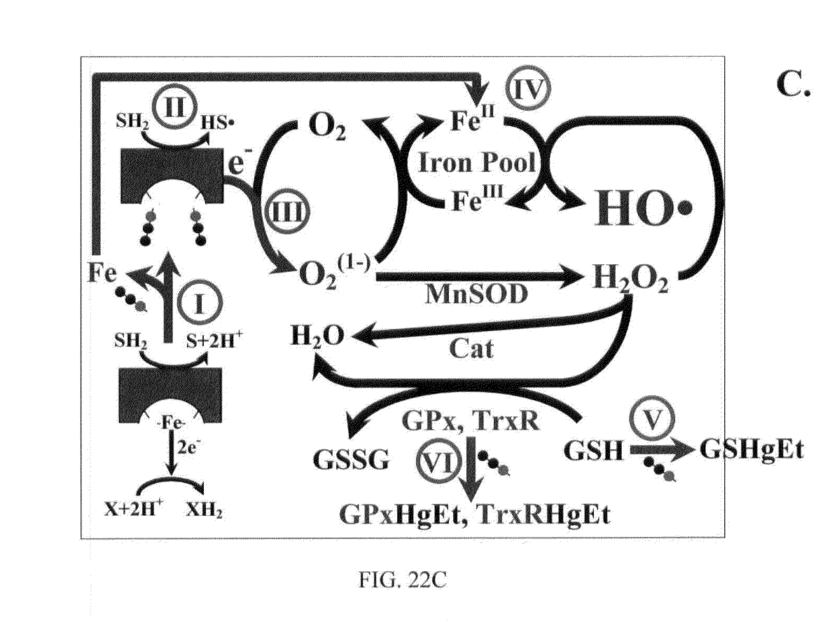

Based on the work reported here and by others, the inventors have suggested a mechanism for the toxicity of organomercury, which is shown in diagrammatic form in FIG. 22A-FIG. 22C. As a lipophilic cation, ethylmercury will become concentrated inside astrocytes, with respect to the bulk extracellular phase, following the plasma membrane potential of 45 mV (Girouard, et al., 2010), by a factor of 5.6-fold, and cytosolic ethlymercury will partition into the mitochondria by a factor of 1,000-fold, its accumulation driven by the approximate 180 mV mitochondrial membrane potential (Clayton et al., 2005), FIG. 22A. Inside the mitochondria, ethylmercury reacts with iron-sulfur centers, causing the release of iron into the mitochondrial matrix (FIG. 22B). The role of ethlymercury in RSO species formation and tetoxification is shown in FIG. 22C. The iron-sulfur centers of oxidoreductases (e.g., succinate dehydrogenase) when damaged by organomercury not only generate free iron, (I), but also form intra-enzymatic carbon radical species (II) that will react with molecular oxygen to give rise to superoxide, (III). Superoxide can react with either free iron generation the ferrous ion or be dismutated into hydrogen peroxide by the mitochondrial Mn-SOD. Ferrous ion and hydrogen peroxide react to generate the highly oxidizing radical, hydroxyl radical, (IV), a agent implicated in pathology and ageing (Harman, 1956; Harman, 1972). The levels of hydrogen peroxide would be generally lowered by the mitochondrial antioxidants, including glutathione dependent selenol/thio based peroxidases, like GPx and TrxR. However, these enzymes are inhibited by organomercury indirectly by depletion of glutathione, (V), and directly by the capping of the active site selenol/thiol by organomercury, (VI).

Thus, the release of iron catalyzes Fenton/Haber-Weiss chemistry leading to the formation of the highly oxidizing HO. HO. has multiple targets, including sensors of the permeability transition complex and also mtDNA. High levels of HO. cause Mitoposis, leading to cytochrome c release from the mitochondria and the initiation of apoptosis. A consequence of ethylmercury exposure to NHA was damage to the mitochondrial genome. Increases were observed in DNA nicks, breaks and most importantly, in the level of oxidized bases. Mitochondria typically have 150 copies of mtDNA and during aging or with exposure to environmental stressors, the number of error free copies of mtDNA undergoes a decline. According to the free radical/mitochondrial theory of aging (Harman, 1956; Harman, 1972), the production of ROS by mitochondria leads to mtDNA damage and mutations. These, in turn, lead to progressive respiratory chain deficits, which result in yet more ROS production, producing a positive feedback loop. The results in this example suggested that ethylmercury acts as a mitochondrial toxin in human astrocytes.

### REFERENCES

The following references, to the extent that they provide exemplary procedural or other details supplementary to those set forth herein, are specifically incorporated herein in their entirety by express reference thereto:
Abdelilah, S G et al., “Functional expression of IL-9 receptor by human neutrophils from asthmatic donors: role in IL-8 release,” J. Immunol., 166:2768-2774 (2001).
Abramov, A Y et al., “Mechanism of neurodegeneration of neurons with mitochondrial DNA mutations,” Brain, 133(Pt 3):797-807 (2010).
Ahlemeyer, B et al., “Ginkgolic acids induce neuronal death and activate protein phosphatase type-2C,” Eur. J. Pharmacol., 430:1-7 (2001).
Ahmed, Z et al., “Formation of apolipoprotein AI-phosphatidylcholine core aldehyde Schiff base adducts promotes uptake by THP-1 macrophages,” Cardiovasc. Res., 58:712-720 (2003).
Anderson, E and G Y Lee, “The polycystic ovarian (PCO) condition: Apoptosis and epithelialization of the ovarian antral follicles are aspects of cystogenesis in the dehydroepiandrosterone (DHEA)-treated rat model,” Tissue Cell, 29:171-189 (1997).
Andollo, N et al., “Structural and functional preservation of specific sequences of DNA and mRNA in apoptotic bodies from ES cells,” Apoptosis, 10:417-428 (2005).
Arakawa S and BR Kimua, “Kinetic study of the interaction of methylmercury with the Fe₂S₂(SR)₄ cluster of adrenodoxin,” J. Am. Chem. Soc., 102:6847-6849 (1980).
Bagheri-Yarmand, R et al., “Activating transcription factor 4 overexpression inhibits proliferation and differentiation of mammary epithelium resulting in impaired lactation and accelerated involution,” J. Biol. Chem., 278:17421-17429 (2009).
Barregard, L et al., “Toxicokinetics of mercury after long-term repeated exposure to thimerosal-containing vaccine,” Toxicol. Sci., 120(2):499-506 (2011).
Baskin, D S et al., “Quantification of DNase type I ends, DNase type II ends, and modified bases using fluorescently labeled ddUTP, terminal deoxynucleotidyl transferase, and formamidopyrimidine-DNA glycosylase,” BioTechniques, 49(1):505-12 (2010).
Baskin, D S et al., “Thimerosal induces DNA breaks, caspase-3 activation, membrane damage, and cell death in cultured human neurons and fibroblasts,” Toxicol. Sci., 74:361-368 (2003).
Baskin, D S et al., “Quantification and calibration of images in fluorescence microscopy,” Anal. Biochem., 404(2):118-26 (2010).
Bragadin, M et al., “Methylmercury induces the opening of the permeability transition pore in rat liver mitochondria,” J. Inorg. Biochem., 89(1-2):159-62 (2002).
Branco, V et al., “Mercury and selenium interaction in vivo: effects on thioredoxin reductase and glutathione peroxidase,” Free Rad. Biol. Med., 52(4):781-793 (2012).
Calabrese, V et al., “Nitrosative stress, cellular stress response, and thiol homeostasis in patients with Alzheimer's disease,” Antioxid. Redox Signal., 8(11-12):1975-86 (2006).
Canty, A J et al., “Octanol/water partition coefficients as a model system for assessing antidotes for methylmercury(II) poisoning, and for studying mercurials with medicinal applications,” J. Inorg. Biochem., 22(1):65-72 (1984).
Chakravarti, A et al., “Temozolomide-mediated radiation enhancement in glioblastoma: A report on underlying mechanisms,” Clin. Cancer Res., 12:4738-4746 (2006).
Charriant-Marlangue, C and Y Ben-Ari. “A cautionary note on the use of the TUNEL stain to determine apoptosis,” NeuroReport, 7:61-64 (1995).
Ciusani, E et al., “Valproic acid increases the in vitro effects of nitrosureas on human glioma cell lines,” Oncol. Res., 16:453-463 (2007).
Clarke D D and SL Seigel, “Circulation and energy metabolism of the brain in Basic neurochemistry: molecular, cellular and medical aspects,” Lippincott Williams & Wilkins: Philadelphia, Pa., pp. 637-669 (1999).
Clayton, R et al., “Cytochrome c release from rat brain mitochondria is proportional to the mitochondrial functional deficit: implications for apoptosis and neurodegenerative disease,” J. Neurochem., 92(4):840-849 (2005).
Colitti, M and M Farinacci, “Cell turnover and gene activities in sheep mammary glands prior to lambing to involution,” Tissue Cell, 41:326-333 (2009).
Collins, T J, “ImageJ for microscopy,” BioTechniques, 43:25-30 (2007).
Counis, M F and A Torriglia, “Acid DNases and their interest among apoptotic endonucleases,” Biochimie, 88:1851-1858 (2006).
Dagda, R K et al., “Loss of PINK1 function promotes mitophagy through effects on oxidative stress and mitochondrial fission,” J. Biol. Chem., 284(20):13843-55 (2009).
Deka, C et al., “Analysis of fluorescence lifetime and quenching of FITC-conjugated antibodies on cells by phase-sensitive flow cytometry,” Cytometry, 25:271-279 (1996).
Desagher, S et al., “Astrocytes protect neurons from hydrogen peroxide toxicity,” p. 2553-2562 (1996).
Didenko, V V and D S Baskin, “Horseradish peroxidase-driven fluorescent labeling of nanotubes with quantum dots,” BioTechniques, 40:295-302 (2006).
Didenko, V V et al., “Semi-artificial fluorescent molecular machine for DNA damage detection,” Nano. Lett., 4:2461-2466 (2004).
Didenko, V V et al., “Substantial background reduction in ligase-based apoptosis detection using newly designed hairpin oligonucleotide probes,” BioTechniques, 27:1130-1132 (1999).
Didenko, V V et al., “Early necrotic DNA degradation: presence of blunt-ended DNA breaks, 3′ and 5′ overhangs in apoptosis, but only 5′ overhangs in early necrosis,” Am. J. Pathol., 162:1571-1578 (2003).
Didenko, V V et al., “Visualization of irreparable ischemic damage in brain by selective labeling of double-strand blunt-ended DNA breaks,” Molec. Med., 8:818-823 (2002).
Didenko, V V et al., “In situ detection of double-strand DNA breaks with terminal 5′OH groups,” Methods Mol. Biol., 203:153-159 (2002).
Fitch, M E et al., “The BH3 domain is required for caspase-independent cell death induced by Bax and oligomycin,” Cell Death Differ., 7(4):338-49 (2000).
Fox C H et al., “Formaldehyde fixation,” J. Histochem. Cytochem., 33(8):845-853 (1985).
Franco, J L et al., “Methylmercury neurotoxicity is associated with inhibition of the antioxidant enzyme glutathione peroxidase,” Free Radical Biol. Med., 47(4):449-457 (2009).
Frosina, G, “Prophylaxis of oxidative DNA damage by formamidopyrimidine-DNA glycosylase,” Intl. J. Cancer, 119:1-7, 2001.
Frosina, G, “DNA repair and resistance of gliomas to chemotherapy and radiotherapy,” Molec. Cancer Res., 7:989-999 (2009).
Gavrieli, Y et al., “Identification of programmed cell death in situ via specific labeling of nuclear DNA fragmentation,” J. Cell Biol., 119:493-501 (1992).
Gill, R D et al., “Increased resistance to N,N′,N″-triethylenethiophosphoramide (thioTEPA) in cells expressing the Escherichia coli formamidopyrimidine-DNA glycosylase,” Cancer Res., 56:3721-3724 (1996).
Girouard, H et al., “Astrocytic endfoot Ca²⁺and BK channels determine both arteriolar dilation and constriction,” Proc. Natl. Acad. Sci. USA, 107(8):3811-6 (2010).
Glaser, V et al., “Effects of inorganic selenium administration in methylmercury-induced neurotoxicity in mouse cerebral cortex,” Int. J. Develop. Neurosci., 28(7):631-637 (2010).
Harbut, M B et al., “Modulation of the turnover of formamidopyrimidine DNA glycosylase,” Biochemistry, 45:7341-7346 (2006).
Harman, D, “Aging: a theory based on free radical and radiation chemistry,” J. Gerontol., 11(3):298-300 (1956).
Harman, D “The biologic clock: the mitochondria?” J. Am. Geriatr Soc., 20(4):145-7 (1972).
Hensley, K, “Detection of protein carbonyls by means of biotin hydrazide-streptavidin affinity methods,” Methods Mol. Biol., 536:457-62 (2009).
Holley, J E et al., “Peroxiredoxin V in multiple sclerosis lesions: predominant expression by astrocytes,” p. 955-961 (2007).
Huck, S., E. Deveaud, A. Namane, and M. Zouali, “Abnormal DNA methylation and deoxycytosine-deoxyguanine content in nucleosomes from lymphocytes undergoing apoptosis,” FASEB J., 13:1415-1422, 1999.
Ishida, Y et al., “Upregulation of protease-activated receptor-1 in astrocytes in Parkinson disease: astrocyte-mediated neuroprotection through increased levels of glutathione peroxidase,” J. Neuropathol. Exp. Neurol., 65(1):66-77 (2006).
King, T E, “Stoichiometry of labile sulfide, nonheme iron and flavin in reconstitutively active succinate dehydrogenase from heart mitochondria,” Biochem. Biophys. Res. Commun., 16(6):511-515 (1994).
Kobune, M et al., “Retrovirus-mediated expression of the base excision repair proteins, formamidopyrimidine DNA glycosylase or human oxoguanine DNA glycosylase, protects hematopoietic cells from N,N′,N″-triethylenethiophosphoramide (thioTEPA)-induced toxicity in vitro and in vivo,” Cancer Res., 61:5116-5125 (2001).
Korshunov, S S et al., “High protonic potential actuates a mechanism of production of reactive oxygen species in mitochondria,” FEBS Lett, 416(1):15-8 (1997).
Koval, V V et al., “Real-time studies of conformational dynamics of the repair enzyme E. coli formamidopyrimidine-DNA glycosylase and its DNA complexes during catalytic cycle,” Mutat. Res./Fund. Molec. Mech. Mutagen., 685(1-2):3-10 (2010).
Kow and Dare, “Detection of abasic sites and oxidative DNA base damage using an ELISA-like assay,” Methods, 22(2):164-9 (2000).
Kralj, M and N Pipan, “Degradation processes in different functional states of the mouse mammary gland,” Periodicum Biologorum, 97:201-206 (1995).
Krishnamurthy, N et al., “Unusual structural features of hydantoin lesions translate into efficient recognition by Escherichia coli Fpg,” Biochemistry, 46(33):9355-65 (2007).
Labat-Moleur F et al., “TUNEL apoptotic cell detection in tissue sections: critical evaluation and improvement,” J. Histochem. Cytochem., 46(3):327-34 (1998).
Lacher, M D et al., “Role of DDC-4/sFRP-4, a secreted frizzled-related protein, at the onset of apoptosis in mammary involution,” Cell Death Different., 10:528-538 (2003).
Lartigue, L et al., “An intracellular wave of cytochrome c propagates and precedes Bax redistribution during apoptosis,” J. Cell Sci., 121:3515-3523 (2008).
LeBel, C P et al., “Deferoxamine inhibits methyl mercury-induced increases in reactive oxygen species formation in rat brain,” Toxicol. Appl. Pharmacol., 112(1):161-5 (1992).
Liddell, J. R., et al., Astrocytes retain their antioxidant capacity into advanced old age. Glia, 58(12):1500-9 (2010).
Lorenz, B and HC Schroder, “Mammalian intestinal alkaline phosphatase acts as highly active exopolyphosphatase,” Biochim. Biophys. Acta Prot. Struct. Molec. Enzymol., 1547:254-261 (2001).
Lossi, L et al., “Cell death and proliferation in acute slices and organotypic cultures of mammalian CNS,” Prog. Neurobiol., 88(4):221-45 (2009).
Margoliash, E and N. Frohwirt “Spectrum of horse-heart cytochrome c,” Biochem. J., 71:570-572 (1959).
Mason, R P et al., “Uptake, toxicity, and trophic transfer of mercury in a coastal diatom,” Environ. Sci. Technol., 30(6):1835-1845 (1996).
Mimura, J et al., “Nrf2 regulates NGF mRNA induction by carnosic acid in T98G glioblastoma cells and normal human astrocytes,” J. Biochem., 150(2):209-17 (2011).
Mori, N et al., “Comparative study of activities in reactive oxygen species production/defense system in mitochondria of rat brain and liver, and their susceptibility to methylmercury toxicity,” Arch. Toxicol., 81(11):769-76 (2007).
Naganuma, A et al., “Overexpression of manganese-superoxide dismutase prevents methylmercury toxicity in HeLa cells,” Life Sci., 62(12):157-61 (1998).
Negoescu A et al., “Importance of DNA fragmentation in apoptosis with regard to TNEL specificity,” Biomed. Pharmacother., 52(6):252-8 (1998).
Negoescu A et al., “In situ apoptotic cell labeling by the TUNEL method: improvement and evaluation on cell preparations,” J. Histochem. Cytochem., 44(9):959-68
(1996).
O'Connor, T R and J Laval, “Physical association of the 2,6-diamino-4-hydroxy-5N-formamidopyrimidine-DNA glycosylase of Escherichia coli and an activity nicking DNA at apurinic/apyrimidinic sites,” Proc. Natl. Acad. Sci. USA, 86:5222-5226 (1989).
Pawley, Handbook of Biological Confocal Microscopy, 3rd edition, Springer, New York, N.Y., 2006.
Pollack, I F et al., “Potent topoisomerase I inhibition by novel silatecans eliminates glioma proliferation in vitro and in vivo,” Cancer Res., 59:4898-4905 (1999).
Power, J and P Blumbergs, “Cellular glutathione peroxidase in human brain: cellular distribution, and its potential role in the degradation of Lewy bodies in Parkinson's disease and dementia with Lewy bodies,” Acta Neuropathologica, 117(1):63-73 (2009).
Reagents for Modifying Aldehydes and Ketones—Section 3.3 The Molecular Probes® Handbook—11th Edition (2000).
Reme, C E et al., “Apoptotic cell death in retinal degenerations,” Prog. Retin. Eye Res., 17:443-464 (1998).
Rich, P R, “A perspective on Peter Mitchell and the chemiosmotic theory,” J. Bioenerg. Biomembr., 40(5):407-10 (2008).
Rohrdanz, E et al., Brain Res, 900(1):128-136 (2001).
Romijn, R J et al., “Double immunolabeling of neuropeptides in the human hypothalamus as analyzed by confocal laser scanning fluorescence microscopy,” J. Histochem. Cytochem., 47(2):229-236, 1999.
Roos, W P and B Kaina, “DNA damage-induced cell death by apoptosis,” Trends Molec. Med., 12(12):440-450 (2006).
Ropolo, M et al., “Accelerated repair and reduced mutagenicity of oxidative DNA damage in human bladder cells expressing the E. coli FPG protein,” Intl. J. Cancer, 118:1628-1634 (2006).
Samai, M et al., “Comparison of the effects of the superoxide dismutase mimetics EUK-134 and tempol on paraquat-induced nephrotoxicity,” Free Radical Biol. Med., 43:528-534 (2007).
Sarafian, T A et al., “Cellular antioxidant properties of human natural killer enhancing factor B,” Free Radic. Res., 26(3):281-9 (1997).
Scott, C W et al., “Novel small molecule inhibitors of caspase-3 block cellular and biochemical features of apoptosis,” J. Pharmacol. Exp. Ther., 304(1):433-40 (2003).
Setsukinai, K et al., “Development of Novel Fluorescence Probes That Can Reliably Detect Reactive Oxygen Species and Distinguish Specific Species. p. 3170-3175 (2003).
Shanker, G et al., “Modulatory effect of glutathione status and antioxidants on methylmercury-induced free radical formation in primary cultures of cerebral astrocytes,” Molec. Brain Res., 137(12):11-22 (2005).
Sharonov, G V et al., “Comparative analysis of pro-apoptotic activity of cytochrome c mutants in living cells,” Apoptosis, 10:797-808 (2005).
Sharpe, M A et al., “Nitric oxide and Fenton/Haber-Weiss chemistry: nitric oxide is a potent antioxidant at physiological concentrations,” J. Neurochem., 87(2):386-394 (2003).
Silvani, A et al., “Cisplatinum and BCNU chemotherapy in primary glioblastoma patients,” J. Neurooncol., 94(1):57-62 (2009).
Skulachev, V P, “Mitochondrial physiology and pathology; concepts of programmed death of organelles, cells and organisms,” Mol. Aspects. Med., 20(3):139-84 (1999).
Smith, M A et al., “Cytochemical demonstration of oxidative damage in Alzheimer disease by immunochemical enhancement of the carbonyl reaction with 2,4-dinitrophenylhydrazine,” J. Histochem. Cytochem., 46:731-736 (1998).
Solier, S and Y Pommier, “The apoptotic ring: A novel entity with phosphorylated histones H2A.X and H2B and activated DNA damage response kinases,” Cell Cycle, 8:1853-1859 (2009).
Song, L et al., “Photobleaching kinetics of fluorescein in quantitative fluorescence microscopy,” Biophys. J., 68:2588-2600 (1995).
Speit, G et al., “Sensitivity of the FPG protein towards alkylation damage in the comet assay,” Toxicol. Lett., 146:151-158 (2004).
Stewart, V C et al., “Astrocyte-derived nitric oxide causes both reversible and irreversible damage to the neuronal mitochondrial respiratory chain,” J. Neurochem., 75(2):694-700 (2000).
Stewart, V C et al., “Preservation of extracellular glutathione by an astrocyte derived factor with properties comparable to extracellular superoxide dismutase,” J. Neurochem., 83(4):984-991 (2002).
Storrie, B et al., “Using quantitative fluorescence microscopy to probe organelle assembly and membrane trafficking,” Methods Mol. Biol., 457:179-192 (2008).
St-Pierre, J et al., “Topology of superoxide production from different sites in the mitochondrial electron transport chain,” J. Biol. Chem., 277(47):44784-44790 (2002).
Suneja, T and D V Belsito, “Thimerosal in the detection of clinically relevant allergic contact reactions,” J. Am. Acad. Dermatol., 45(1):23-27 (2001).
Suzuki, T et al., “Recent advances in fluorescent labeling techniques for fluorescence microscopy,” Acta Histochemica et Cytochemica, 40:131-139 (2007).
Swedlow, J R, “Quantitative fluorescence microscopy and image deconvolution,” Methods Cell Biol., 81:447-465 (2007).
Talhavini, M and T D Z Atvars, “Dye-polymer interactions controlling the kinetics of fluorescein photobleaching reactions in poly(vinyl alcohol),” J. Photochem. Photobiol. A: Chem., 114:65-73 (1998).
Tatarczuch, L et al., “Involution of the sheep mammary gland,” J. Anat., 190:405-416 (1997).
Tataurov et al., “Predicting ultraviolet spectrum of single stranded and double stranded deoxyribonucleic acids,” Biophys. Chem., 133(1-3):66-70 (2008).
Torriglia, A and C. Lepretre, “LEI/L-DNase II: interplay between caspase-dependent and independent pathways,” Frontiers Biosci., 14:4836-4847 (2009).
Vinken, M et al., “DNA methyltransferase 3a expression decreases during apoptosis in primary cultures of hepatocytes,” Toxicol. In Vitro, 24(2):445-451 (2010).
Walker, N I et al., “Cell death by apoptosis during involution of the lactating breast in mice and rats,” Amer. J. Anat., 185:19-32 (1989).
Waters, J C, “Accuracy and precision in quantitative fluorescence microscopy,” J. Cell Biol., 185:1135-1148 (2009).
White, B et al., “Oscillating formation of 8-oxoguanine during DNA oxidation,” J. Am. Chem. Soc., 125(22):6604-5 (2003).
Whiteman, M et al., “Detection and measurement of reactive oxygen intermediates in mitochondria and cells,” Methods Mol. Biol., 476:28-49 (2009).
Wolf, D E., “Fundamentals of fluorescence and fluorescence microscopy,” Methods Cell Biol., 81:63-91 (2007).
Wu, M et al., “Protection of human lung cells against hyperoxia using the DNA base excision repair genes hOgg1 and Fpg,” Amer. J. Respir. Crit. Care Med., 166:192-199 (2002).
Xu, Y and MA Villalona-Calero, “Irinotecan: mechanisms of tumor resistance and novel strategies for modulating its activity,” Ann. Oncol., 13:1841-1851 (2002).
Xu, Y et al., “Protection of mammalian cells against chemotherapeutic agents thiotepa, 1,3-N,N-bis(2-chloroethyl)-N-nitrosourea, and mafosfamide using the DNA base excision repair genes Fpg and Ogg1: Implications for protective gene therapy applications,” J. Pharmacol. Exp. Ther., 296:825-831 (2001).
Yasuda, M et al., “Apoptotic cells in the human endometrium and placental villi: Pitfalls in applying the TUNEL method,” Arch. Histol. Cytol., 58:185-190 (1995).
Yin, Z et al., “Methylmercury-induced alterations in astrocyte functions are attenuated by ebselen,” Neurotoxicology, 32(3):291-299 (2011).
Ying-Hui, H E et al., “Escherichia coli FPG and human OGG1 reduce DNA damage and cytotoxicity by BCNU in human lung cells,” Amer. J. Physiol. Lung Cell. Molec. Physiol., 282:L50-L55 (2002).
Zwier, J M et al., “Image calibration in fluorescence microscopy,” J. Microscopy, 216:15-24, 2004.
Zwier, J M et al., “Absolute and relative quantification and calibration for sectioning fluorescence microscopy using standardized uniform fluorescent layers and SIPchart based correction procedures,” Progr. Biomed. Optics Imaging 8 [SPIE Proceedings, vol., 6443] (2007).
Zwier, J M et al., “Quantitative image correction and calibration for confocal fluorescence microscopy using thin reference layers and SIPchart-based calibration procedures,” J. Microscopy, 231:59-69 (2008).

All of the compositions and methods disclosed and claimed herein can be made and executed without undue experimentation in light of the present disclosure. While the compositions and methods of this invention have been described in terms of exemplary embodiments, it will be apparent to those of skill in the art that variations may be applied to the composition, methods and in the steps or in the sequence of steps of the method described herein without departing from the concept, spirit and scope of the invention. More specifically, it will be apparent that certain agents that are both chemically- and physiologically-related may be substituted for the agents described herein while the same or similar results would be achieved. All such similar substitutes and modifications apparent to those of ordinary skill in the art are deemed to be within the spirit, scope and concept of the invention as defined herein.

What is claimed is:

1. A tissue phantom comprising gelatin that is operably linked to at least a first detection moiety.

2. The tissue phantom of claim 1, wherein the gelatin is porcine skin gelatin.

3. The tissue phantom of claim 1, wherein the gelatin is covalently-linked to the first detection moiety using one or more amine-reactive crosslinking agents attached to a first calibration standard.

4. The tissue phantom of claim 3, wherein the first calibration standard comprises a chromophoric dye, a first fluorophoric dye, a first oligonucleotide, a first protein, a first peptide, a first enzyme, a first antibody, or any combination thereof.

5. The tissue phantom of claim 1, wherein the amine-reactive crosslinking agent is suberic acid bis(N-hydrosuccinimide ester, or a derivative or analog thereof.

6. The tissue phantom of claim 1, wherein the concentration of gelatin is about 7.5% to about 20%.

7. The tissue phantom of claim 4, wherein the first peptide, the first protein, the first enzyme, or the first antibody may be directly crosslinked to the gelatin by paraformaldehyde fixation.

8. The tissue phantom of claim 1, wherein the first calibration standard is adapted and configured for use in UV, visible, fluorescence, or epifluorescence microscopy.

9. The tissue phantom of claim 1, adapted and configured for use on a microscope slide or in one or more wells of a multi-well assay plate.

10. The tissue phantom of claim 9, wherein the microscope slide comprises a population of distinct tissue phantoms each of which comprises a different, known quantity of the first calibration standard.

11. The tissue phantom of claim 1, further comprising a second distinct detection moiety, or a second distinct calibration standard.

12. The tissue phantom of claim 9, wherein the presence of the population of distinct tissue phantoms comprising differing, but known quantities, of the first calibration standard permits quantitation of one or more selected compounds of interest within a specimen, a sample, a tissue, a cell, or any combination.

13. The tissue phantom of claim 4, wherein the first calibration standard comprises 6-FITC.

14. The tissue phantom of claim 4, wherein the first calibration standard comprises a Cy3-labeled protein, a Cy3-labeled antibody or antigen binding fragment, or any combination thereof.

15. An article of manufacture comprising the tissue phantom of claim 1.

16. The article of manufacture of claim 16, comprising a plurality of distinct tissue phantoms, each of which includes a distinct, known amount of the first calibration standard, adapted and configured to facilitate the generation of a standard curve to quantitate one or more selected compounds of interest within a specimen, a sample, a tissue, a cell, or any combination thereof.

17. The article of manufacture of claim 15, defined as a cuvette, a cell culture plate, a microscope slide, a microtiter dish, or a multi-well assay plate.

18. A method of modifying a terminal deoxynucleotidyl transferase nick end labeling assay, comprising substituting a 3′ dideoxy UTP substrate for a dUTP substrate under conditions effective to permit quantitation of the —OH 3′ ends present in an assayed biological sample suspected of containing a population of nucleic acids.

19. The method of claim 18, wherein the presence of the ddUPT permits the stoichiometric addition of a single label at each original —OH 3′ end present in the sample.

20. A method of quantitating —PO₄ 3′ ends in a nucleic acid molecule, comprising using calf intestinal alkaline phosphatase to convert the —PO₄ 3′ ends to —OH 3′ ends, and then assaying the converted —OH 3′ ends using a ddTUNEL assay.

21. The method of claim 20, further comprising oxidizing or acetylating at least a first nucleobase of the polynucleotide molecule, comprising contacting the sample with an effective amount of formamidopyrimidine DNA glycosylase (Fpg).

22. The method of claim 21, comprising the further step of treating the resulting oxo-species by borohydride reduction or by derivatization with 2,4-dinitrophenyl hydrazine (DNP-H).

23. A method of visualizing reactive oxygen species damage within a biological cell, wherein one or more 2,4-dinitrophenyl hydrazine-derivatized oxo-species are localized in the cell by detecting the presence of a labeled anti-DNP antibody.

24. A method of interrogating cell death within one or more cells present in a biological sample, comprising performing one or more ddTUNEL, CIAP-ddTUNEL, Fpg-ddTUNEL assays using a signal-calibrated tissue phantom in accordance with claim 1, under conditions effective to monitor the level of apoptosis in one or more such cells.

25. The method of claim 24, wherein the step of monitoring includes epifluorescence microscopy.

26. The method of claim 25, wherein the epifluorescence microscopy is adapted and configured with one or more optical filter blocks that include: a) a DAPI channel, b) an FITC channel, c) a Texas Red channel, or any combination thereof.

27. The method of claim 25, wherein a) the DAPI channel is adapted and configured for the detection of a biological probe that comprises 4′,6-diamidino-2-phenylindole; b) the FITC channel is adapted and configured for the detection of a biological probe that comprises an amine-reactive fluorescein derivative; or c) the Texas Red channel is adapted and configured for the detection of a biological probe that comprises sulforhodamine 101 acid chloride or a derivative or analog thereof.

28. The method of claim 27, wherein the amine-reactive fluorescein derivative is fluorescein isothiocyanate, Alexa Fluor 488, or DyLight488, or any combination thereof.


SEQUENCE LISTING (32 sequences)

SEQ ID NO: 1 - DNA, 16 bp, Artificial Sequence
  Synthetic Oligonucleotide
ggtctggatccagcgc

SEQ ID NO: 2 - DNA, 16 bp, Artificial Sequence
  Synthetic Oligonucleotide
ccagacctaggtcgcg

SEQ ID NO: 3 - DNA, 14 bp, Artificial Sequence
  Synthetic Oligonucleotide
gctgaattcagacc

SEQ ID NO: 4 - DNA, 14 bp, Artificial Sequence
  Synthetic Oligonucleotide
cgacttaagtctgg

SEQ ID NO: 5 - DNA, 13 bp, Artificial Sequence
  Synthetic Oligonucleotide
ggtctgatccgct

SEQ ID NO: 6 - DNA, 12 bp, Artificial Sequence
  Synthetic Oligonucleotide
ccagactaggcg

SEQ ID NO: 7 - DNA, 16 bp, Artificial Sequence
  Synthetic Oligonucleotide
gcgctgaattcagacc

SEQ ID NO: 8 - DNA, 17 bp, Artificial Sequence
  Synthetic Oligonucleotide
acgcgacttaagtctgg

SEQ ID NO: 9 - DNA, 21 bp, Artificial Sequence
  Synthetic Oligonucleotide
nngcgcaagcgtcgcgcaann

SEQ ID NO: 10 - DNA, 21 bp, Artificial Sequence
  Synthetic Oligonucleotide
nncgcgttcgcagcgtgttnn

SEQ ID NO: 11 - DNA, 21 bp, Artificial Sequence
  Synthetic Oligonucleotide
nngctcaagcgtcgcgcaann

SEQ ID NO: 12 - DNA, 21 bp, Artificial Sequence
  Synthetic Oligonucleotide
nncgcgttcgcagcgtgttnn

SEQ ID NO: 13 - DNA, 20 bp, Artificial Sequence
  Synthetic Oligonucleotide
nngccaagcgtcgcgcaann

SEQ ID NO: 14 - DNA, 20 bp, Artificial Sequence
  Synthetic Oligonucleotide
yycgcgttcgcagcggttyy

SEQ ID NO: 15 - DNA, 20 bp, Artificial Sequence
  Synthetic Oligonucleotide
nngccaagcgtcgcgcaann

SEQ ID NO: 16 - DNA, 20 bp, Artificial Sequence
  Synthetic Oligonucleotide
yycgcgttcgcagcggttyy

SEQ ID NO: 17 - DNA, 21 bp, Artificial Sequence
  Synthetic Oligonucleotide
nngcgcaagcgtcgcgcaann

SEQ ID NO: 18 - DNA, 21 bp, Artificial Sequence
  Synthetic Oligonucleotide
yycgcgttcgcagcgrgttyy

SEQ ID NO: 19 - DNA, 21 bp, Artificial Sequence
  Synthetic Oligonucleotide
nngcrcaagcgtcgcgcaann

SEQ ID NO: 20 - DNA, 21 bp, Artificial Sequence
  Synthetic Oligonucleotide
yycgcgttcgcagcgtgttyy

SEQ ID NO: 21 - DNA, 20 bp, Artificial Sequence
  Synthetic Oligonucleotide
nngccaagcgtcgcgcaann

SEQ ID NO: 22 - DNA, 21 bp, Artificial Sequence
  Synthetic Oligonucleotide
yycgcgttcgcagcgrgttyy

SEQ ID NO: 23 - DNA, 20 bp, Artificial Sequence
  Synthetic Oligonucleotide
nngccaagcgtcgcgcaann

SEQ ID NO: 24 - DNA, 21 bp, Artificial Sequence
  Synthetic Oligonucleotide
yycgcgttcgcagcgrgttyy

SEQ ID NO: 25 - DNA, 10 bp, Artificial Sequence
  Synthetic Oligonucleotide
yyyyyyyyyy

SEQ ID NO: 26 - DNA, 10 bp, Artificial Sequence
  Synthetic Oligonucleotide
nnnnnnnnnn

SEQ ID NO: 27 - DNA, 10 bp, Artificial Sequence
  Synthetic Oligonucleotide
yyyyyyyyyy

SEQ ID NO: 28 - DNA, 11 bp, Artificial Sequence
  Synthetic Oligonucleotide
nnnnnnnnnnn

SEQ ID NO: 29 - DNA, 10 bp, Artificial Sequence
  Synthetic Oligonucleotide
yyyyyyyyyy

SEQ ID NO: 30 - DNA, 11 bp, Artificial Sequence
  Synthetic Oligonucleotide
nnnnnnnnnnn

SEQ ID NO: 31 - DNA, 11 bp, Artificial Sequence
  Synthetic Oligonucleotide
yyyyyyyyyyn

SEQ ID NO: 32 - DNA, 11 bp, Artificial Sequence
  Synthetic Oligonucleotide
nnnnnnnnnnn